(19) United States
(12) Patent Application Publication
(10) Pub. No.: US 2021/0085897 A1
(43) Pub. Date: Mar. 25, 2021

(54) MEDICAMENT DISPENSER

(71) Applicant: LUPIN INC., Coral Springs, FL (US)

(72) Inventors: Abhishek GUPTA, Coral Springs, FL (US); Imran SHAIKH, Coral Springs, FL (US); Xian-Ming ZENG, Coral Springs, FL (US)

(73) Assignee: LUPIN INC., Coral Springs, FL (US)

(21) Appl. No.: 17/044,662

(22) PCT Filed: Apr. 5, 2019
(86) PCT No.: PCT/US2019/026038

§ 371 Date: Oct. 1, 2020

Related U.S. Application Data
(60) Provisional application No. 62/654,009, filed on Apr. 6, 2018.

(51) Int. Cl. A61M 15/00

(52) U.S. Cl. CPC A61M 15/0043; A61M 15/0026; A61M 15/007; A61M 2202/064; A61M 15/0051; A61M 15/0055; A61M 15/008

(57) ABSTRACT

A medicament dispenser for dispensing medicament comprising: an assembly comprising: at least one peelable medicament carrier carrying multiple distinct medicament portions; a medicament carrier advancing mechanism; a flow channel; and a hinged cover for the flow channel.

### CROSS REFERENCE TO RELATED APPLICATIONS

This application is being filed on 5 Apr. 2019, as a PCT International patent application, and claims priority to a U.S. Provisional Application No. 62/654,009 filed on Apr. 6, 2018. The entirety of the aforementioned application is hereby incorporated herein by reference.

### FIELD

The present disclosure relates to a medicament dispenser for dispensing medicament. The dispensing mechanism can include three steps to operate the medicament dispenser: Open, Dispense and Close.

### BACKGROUND

### Description of the Related Art

Medicaments are administered to a patient by inhalation using dry powder dispenser devices. Such devices are often referred to as dry powder inhalers (DPI), and are used for the treatment and prophylaxis of respiratory diseases, including, but not limited to asthma, chronic obstructive pulmonary disease (COPD), bronchitis, lung infections, interstitial pulmonary fibrosis (IPF), pulmonary arterial hypertension (PAH), cystic fibrosis, Non-CF bronchiectasis and lung transplant.

### SUMMARY

Generally described, dry powder inhalers carry medicament in reservoirs, capsules or blisters packages, and can otherwise carry medicament. The medicament carrier can include a blister strip containing a number of discrete doses of powdered medicament. Such devices can contain a mechanism of accessing these doses, either piercing means or means to peel a lid sheet away from a base sheet. The powdered medicament can then be accessed and inhaled. Such a mechanism may also be used for dispensing medicament in tablets, capsules, pellets or pucks form wherein peeling away the lid sheet from the base sheet reveals a particular medicament form such as tablet for removal and subsequent consumption, particularly for oral administration.

U.S. Pat. No. 5,873,360 discloses an inhalation device, wherein a cover is pivotal between an open position in which the mouthpiece is exposed and a closed position in which it is not. In operation, the user moves the cover to its open position and then presses on the finger tab of the lever to cause it to move as the lever pivots. The lever associated with the finger tab, actuates the gear mechanism as the finger tab is slid from end to end. Therefore, the blister strip is advanced, one blister is opened, and one dose of the dry powder medicament contained in the blister cavity, become ready for the inhalation. The cover is held stationary as the body is rotated anticlockwise. As the cover of the device is not directly connected to the lever mechanism, the user needs to first move the cover and then lever and thus more number of steps are required to operate the device.

U.S. Pat. No. 8,161,968 discloses a medicament dispenser for containing plural elongate form medicament carriers, wherein said medicament dispenser comprises a movable cover for the outlet which is movable from an at rest position, in which the cover covers the outlet, to a primed position and then an actuated position to uncover the outlet, and couples to the dispensing mechanism such that: movement of said cover from the primed position to the actuated position actuates one or more components of the dispensing mechanism, and movement of said cover from the at rest position to the primed position does not actuate said one or more components of the dispensing mechanism.

U.S. Pat. No. 8,746,242 discloses a medicament dispenser for use with at least one medicament carrier carrying multiple distinct medicament portions, said medicament dispenser comprising (a) a dispensing mechanism actuable for dispensing the distinct medicament portions carried by said at least one medicament carrier; (b) a mouthpiece; and (c) a cover for said mouthpiece, said cover being movably mounted to the dispenser for sequential movement from a first position, in which said mouthpiece is covered, to a second position, in which said mouthpiece is part-uncovered, to a third position in which said mouthpiece is uncovered; wherein said cover is adapted to couple with said dispensing mechanism such that movement of the cover from the first position to the second position does not result in actuation of the dispensing mechanism, but any further movement of the cover from the second position to the third position results in actuation of the dispensing mechanism.

In operation, it is potentially desirable that dispensing mechanism comprises minimum number of steps to operate medicament dispenser. It is also potentially desirable that dispensing mechanism offers a continuous movement of the cover and without any second/intermediate position during opening and closing processes so as to avoid any confusion to the user. Further, increase in number of steps will increase complexity and cost associated with development of inhalation device.

In order to overcome such problems, the present disclosure describes a medicament dispenser which is cost-effective and which is used for dispensing medicament in reliable and easy way. Thus, the user actuates the mechanism with minimum possible steps to advance the medicament carrier thereby causing the next discrete dose of medicament to be made available for dispensing. The present disclosure further relates to the cover pivotally attached to the assembly of the dispenser by a hinge. By hinging the cover to the assembly in that way the user can simply and conveniently open the cover by gripping the medicament dispenser with one hand, for example by placing a thumb on the cover and fingers on the back of the assembly. This construction avoids many of the real difficulties that some users experience when trying to open commercially available inhalers.

According to one aspect of the disclosure relate to a medicament dispenser for dispensing medicament comprising: a) an assembly comprising: i) at least one medicament carrier carrying multiple distinct medicament portions; ii) a medicament carrier advancing mechanism; iii) a flow channel; and b) a cover for the flow channel.

Another aspect of the disclosure relate to a medicament dispenser for dispensing medicament comprising: a) an assembly comprising: i) at least one medicament carrier carrying multiple distinct medicament portions; ii) a medicament carrier advancing mechanism; iii) a flow channel; and b) a hinged cover for the flow channel.

Another aspect of the disclosure relate to a medicament dispenser for dispensing medicament comprising: a) an assembly comprising: i) at least one peelable medicament carrier carrying multiple distinct medicament portions; ii) a medicament carrier advancing mechanism; iii) a flow channel; and b) a hinged cover for the flow channel.

Further aspect of the disclosure relate to a medicament dispenser for dispensing medicament comprising: a) an assembly comprising: i) at least one peelable medicament carrier carrying multiple distinct medicament portions; ii) a medicament carrier advancing mechanism; iii) a flow channel; iv) a driver gear; and b) a hinged cover for the flow channel.

Further aspect of the disclosure relate to a medicament dispenser for dispensing medicament comprising: a) an assembly comprising: i) at least one peelable medicament carrier carrying multiple distinct medicament portions; ii) a medicament carrier advancing mechanism; iii) a flow channel; iv) a driver gear; and b) a hinged cover for the flow channel, wherein the hinged cover is adapted to couple with the driver gear and the medicament carrier advancing mechanism.

Some aspects of the disclosure relate to a medicament dispenser for dispensing medicament comprising: a) an assembly comprising: i) at least one peelable medicament carrier carrying multiple distinct medicament portions; ii) a medicament carrier advancing mechanism; iii) a flow channel; iv) a driver gear; and b) a hinged cover for the flow channel, wherein the hinged cover being movably mounted to the dispenser for sequential movement from an initial position, in which said flow channel is covered, to a dispense position, in which said flow channel is uncovered.

Another aspect of the disclosure relate to a medicament dispenser for dispensing medicament comprising: a) an assembly comprising: i) at least one peelable medicament carrier carrying multiple distinct medicament portions; ii) a medicament carrier advancing mechanism; iii) a flow channel; iv) a driver gear; and b) a hinged cover for said flow channel, wherein said hinged cover is adapted to couple with said medicament carrier advancing mechanism such that the movement of the hinged cover from an initial position rotates the driver gear to actuate the medicament carrier advancing mechanism.

Another aspect of the disclosure relate to a medicament dispenser for dispensing medicament comprising: a) an assembly comprising: i) at least one peelable medicament carrier carrying multiple distinct medicament portions; ii) a mechanism of actuation for dispensing the distinct medicament portions; iii) a flow channel; iv) a driver gear; and b) a hinged cover for said flow channel, wherein the mechanism of actuation provides continuous movement of hinged cover without any idling position between an initial position and a dispense position.

Yet another aspect of the disclosure relate to a medicament dispenser for dispensing medicament comprising: a) an assembly comprising: i) at least one peelable medicament carrier carrying multiple distinct medicament portions; ii) a medicament carrier advancing mechanism iii) a flow channel; and b) a hinged cover for the flow channel, wherein the flow channel is covered by the hinged cover in an initial position, and fully revealed when the hinged cover is in a dispense position.

Some aspects of the disclosure relate to a medicament dispenser for dispensing medicament comprising: a) an assembly comprising: i) at least one peelable medicament carrier carrying multiple distinct medicament portions; ii) a medicament carrier advancing mechanism iii) a flow channel; iv) a driver gear; and b) a hinged cover for the flow channel, wherein the flow channel is covered by the hinged cover in an initial position, and fully revealed when the hinged cover is in a dispense position.

Some aspects of the disclosure relate to a medicament dispenser for dispensing medicament comprising: a) an assembly comprising: i) at least one peelable medicament carrier carrying multiple distinct medicament portions; ii) a mechanism of actuation for dispensing the distinct medicament portions; iii) a flow channel; iv) a mouthpiece iv) a driver gear; and b) a hinged cover for said mouthpiece comprises, wherein the mechanism of actuation provides continuous movement of the hinged cover such that there is no intermediate position for hinged cover between an initial position and a dispense position.

Some aspects of the disclosure relate to a medicament dispenser for dispensing medicament comprising: a) an assembly comprising: i) at least one peelable medicament carrier carrying multiple distinct medicament portions; ii) a medicament carrier advancing mechanism for actuating the distinct medicament portions; iii) a flow channel; iv) a driver gear; and b) a hinged cover for said flow channel, wherein the coupling of the hinged cover with the medicament carrier advancing mechanism is adapted such the movement of the cover from an initial position to a dispense position results in actuation of dispensing mechanism without any idle movement of the hinged cover.

Some aspects of the disclosure relate to a medicament dispenser for dispensing medicament comprising: a) an assembly comprising: i) at least one peelable medicament carrier carrying multiple distinct medicament portions; ii) a medicament carrier advancing mechanism; iii) a flow channel; iv) a driver gear; and b) a hinged cover for said flow channel, wherein the dispenser is an inhalation device.

Some aspects of the disclosure relate to a medicament dispenser for dispensing medicament comprising: a) an assembly comprising: a medicament carrier; a flow channel; a medicament carrier advancing mechanism; a driver gear; and b) a hinged cover, wherein the hinged cover is movable form an initial position, in which the hinged cover covers said flow channel, to a dispense position to uncover the flow channel, and the movement of the hinged cover coupled to said medicament carrier advancing mechanism by the driver gear that are arranged such that movement of the hinged cover from the initial position to the dispense position results in actuation of the medicament carrier advancing mechanism by the driver gear thereby advances the medicament carrier to align that with the flow channel, and through which a user can access the medicament from said flow channel, and movement of the hinged cover from the dispense position to the initial position results in rotation of the driver gear and without advancing the medicament carrier.

Another aspect of the disclosure relate to a medicament dispenser for dispensing medicament comprising: a) an assembly comprising: i) at least one peelable medicament carrier carrying multiple distinct medicament portions; ii) a medicament carrier advancing mechanism; iii) a flow channel; iv) a driver gear; v) an interim driver gear; and b) a hinged cover for the flow channel.

Another aspect of the disclosure relate to a medicament dispenser for dispensing medicament comprising: a) an assembly comprising: i) at least one peelable medicament carrier carrying multiple distinct medicament portions; ii) a medicament carrier advancing mechanism; iii) a flow channel; iv) a driver gear; v) an interim driver gear; and b) a hinged cover for the flow channel, wherein the hinged cover being movably mounted to the dispenser for sequential movement from an initial position, in which said flow channel is covered, to a dispense position, in which said flow channel is uncovered.

Further aspect of the disclosure relate to a medicament dispenser for dispensing medicament comprising: a) an assembly comprising: i) at least one peelable medicament carrier carrying multiple distinct medicament portions; ii) a medicament carrier advancing mechanism; iii) a flow channel; iv) a driver gear; v) an interim driver gear; and b) a hinged cover for said flow channel, wherein said hinged cover is adapted to couple with said medicament carrier advancing mechanism such that the movement of the hinged cover from an initial position rotates the driver gear and the interim driver gear to actuate the medicament carrier advancing mechanism.

Further aspect of the disclosure relate to a medicament dispenser for dispensing medicament comprising: a) an assembly comprising: i) at least one peelable medicament carrier carrying multiple distinct medicament portions; ii) a mechanism of actuation for dispensing the distinct medicament portions; iii) a flow channel; iv) a driver gear; v) an interim driver gear; and b) a hinged cover for said flow channel, wherein the mechanism of actuation provides continuous movement of hinged cover without any idling position between an initial position and a dispense position.

Some aspects of the disclosure relate to a medicament dispenser for dispensing medicament comprising: a) an assembly comprising: i) at least one peelable medicament carrier carrying multiple distinct medicament portions; ii) a medicament carrier advancing mechanism iii) a flow channel; iv) a driver gear; v) an interim driver gear; and b) a hinged cover for the flow channel, wherein the flow channel is covered by the hinged cover in an initial position, and fully revealed when the hinged cover is in a dispense position.

Some aspects of the disclosure relate to a medicament dispenser for dispensing medicament comprising: a) an assembly comprising: i) at least one peelable medicament carrier carrying multiple distinct medicament portions; ii) a mechanism of actuation for dispensing the distinct medicament portions; iii) a flow channel; iv) a mouthpiece iv) a driver gear; v) an interim driver gear; and b) a hinged cover for said mouthpiece comprises, wherein the mechanism of actuation provides continuous movement of the hinged cover such that there is no intermediate position for hinged cover between an initial position and a dispense position.

Some aspects of the disclosure relate to a medicament dispenser for dispensing medicament comprising: a) an assembly comprising: i) at least one peelable medicament carrier carrying multiple distinct medicament portions; ii) a medicament carrier advancing mechanism for actuating the distinct medicament portions; iii) a flow channel; iv) a driver gear; v) an interim driver gear; and b) a hinged cover for said flow channel, wherein the coupling of the hinged cover with the medicament carrier advancing mechanism is adapted such the movement of the cover from an initial position to a dispense position results in actuation of dispensing mechanism without any idle movement of the hinged cover.

Any feature, structure, or step disclosed herein can be replaced with or combined with any other feature, structure, or step disclosed herein, or omitted. Further, for purposes of summarizing the disclosure, certain aspects, advantages, and features of the inventions have been described herein. It is to be understood that not necessarily any or all such advantages are achieved in accordance with any particular embodiment of the inventions disclosed herein. No individual aspects of this disclosure are essential or indispensable.

### BRIEF DESCRIPTION OF THE DRAWINGS

FIGS. 1A-1B show different views of one embodiment of the medicament dispenser.

FIG. 2 shows an exploded view of the medicament dispenser shown in FIGS. 1A-1B.

FIGS. 3A-3C show the demonstration of the Open, Dispense, Close mechanism of the medicament dispenser shown in FIGS. 1A-1B.

FIGS. 4A-4C show inner components and working of the medicament dispenser shown in FIGS. 1A-1B.

FIG. 5 shows a partial exploded view of the medicament dispenser shown in FIGS. 1A-1B.

FIGS. 6A-6D show opening and closing of the medicament dispenser shown in FIGS. 1A-1B.

FIGS. 7A-7B show different views of the cover of the medicament dispenser shown in FIGS. 1A-1B.

FIG. 8 shows view of space for the electronics in the cover of the medicament dispenser shown in FIGS. 1A-1B.

FIGS. 9A-9B show different views of an embodiment of the blister cuvette that is compatible with the medicament dispenser described herein.

FIG. 10 shows an embodiment of the cuvette ratchet that is compatible with the medicament dispenser described herein.

FIG. 11 shows inner view of the base cover of the medicament dispenser shown in FIGS. 1A-1B.

FIG. 12 shows outer view of the base cover of the medicament dispenser shown in FIGS. 1A-1B.

FIG. 13 shows outer view of the top cover of the medicament dispenser shown in FIGS. 1A-1B.

FIG. 14 shows inner view of the top cover of the medicament dispenser shown in FIGS. 1A-1B.

FIG. 15 shows an example of a driver gear that is compatible with the medicament dispenser described herein.

FIGS. 16A-16B show different views of a winder assembly that is compatible with the medicament dispenser described herein.

FIG. 17 shows an example of an inner housing support of the medicament dispenser shown in FIGS. 1A-1B.

FIGS. 18A-18B show different views of an inner housing assembly of the medicament dispenser shown in FIGS. 1A-1B.

FIG. 19 shows an example of a dose indicator units wheel that is compatible with the medicament dispenser described herein.

FIGS. 20A-20B show different views of a dose indicator transfer wheel that is compatible with the medicament dispenser described herein.

FIGS. 21A-21B show different views of a dose indicator tens wheel that is compatible with the medicament dispenser described herein.

FIG. 22 shows an example of a wrapping wheel and gears that is compatible with the medicament dispenser described herein.

FIG. 23 shows an exploded view of the dose indicator assembly that is compatible with the medicament dispenser described herein.

FIGS. 24A-24C show working of the dose indicator assembly that is compatible with the medicament dispenser described herein.

FIGS. 25A-25B show working of the dose indicator assembly when all dose are exhausted from the medicament dispenser described herein.

FIGS. 26A-26B show different views of an embodiment of the reel of blisters that is compatible with the medicament dispenser described herein.

FIGS. 27A-27C show different views of air inlet vent holes of the medicament dispenser shown in FIGS. 1A-1B.

FIG. 28 shows an example of mouthpiece vent holes that is compatible with the medicament dispenser described herein.

FIGS. 29A-29F show an example of the locking mechanism of the medicament dispenser described herein.

FIGS. 30A-30C show a dotted gripping feature and a wave gripping feature of the cover of the medicament dispenser shown in FIGS. 1A-1B.

FIGS. 31A-31C show different views of another embodiment of the medicament dispenser.

FIG. 32 shows an exploded view of the medicament dispenser shown in FIGS. 31A-31C.

FIGS. 33A-33C show the demonstration of the Open, Dispense, Close mechanism of the medicament dispenser shown in FIGS. 31A-31C.

FIGS. 34A-34C show inner components and working of the medicament dispenser shown in FIGS. 31A-31C.

FIG. 35 shows a partial exploded view of the medicament dispenser shown in FIGS. 31A-31C.

FIGS. 36A-36D show opening and closing of the medicament dispenser shown in FIGS. 31A-31C.

FIGS. 37A-37B show different views of the cover of the medicament dispenser shown in FIGS. 31A-31C.

FIG. 38 shows view of space for the electronics in the cover of the medicament dispenser shown in FIGS. 31A-31C.

FIG. 39 shows inner view of a base cover of the medicament dispenser shown in FIGS. 31A-31C.

FIG. 40 shows outer view of a base cover of the medicament dispenser shown in FIGS. 31A-31C.

FIG. 41 shows outer view of a top cover of the medicament dispenser shown in FIGS. 31A-31C.

FIG. 42 shows inner view of a top cover of the medicament dispenser shown in FIGS. 31A-31C.

FIG. 43 shows inner housing support of the medicament dispenser shown in FIGS. 31A-31C.

FIGS. 44A-44B show different views of the inner housing assembly of the medicament dispenser shown in FIGS. 31A-31C.

FIG. 45 shows an example of a dose indicator wheel that is compatible with the medicament dispenser described herein.

FIG. 46 shows an exploded view of the dose indicator assembly that is compatible with the medicament dispenser described herein.

FIGS. 47A-47B show working of the dose indicator assembly that is compatible with the medicament dispenser described herein.

FIGS. 48A-48B show working of the dose indicator assembly when all dose are exhausted from the medicament dispenser described herein.

FIGS. 49A-49C show different views of air inlet vent holes of the medicament dispenser shown in FIGS. 31A-31C.

FIG. 50 shows an example of mouthpiece vent holes that is compatible with the medicament dispenser described herein.

FIGS. 51A-51B show an example of an extended mouthpiece and cover of the medicament dispenser described herein.

FIGS. 52A-52B show a dotted gripping feature and a wave gripping feature of the medicament dispenser shown in FIGS. 31A-31C.

FIGS. 53A-53D show an example of the locking mechanism of the medicament dispenser shown in FIGS. 31A-31C.

FIGS. 54A-54C show another example of the locking mechanism of the medicament dispenser shown in FIGS. 31A-31C.

FIGS. 55A-55D show another example of the locking mechanism of the medicament dispenser shown in FIGS. 31A-31C.

FIGS. 56A-56D show another example of the locking mechanism of the medicament dispenser shown in FIGS. 31A-31C.

### DETAILED DESCRIPTION

The present disclosure relates to a medicament dispenser for dispensing medicament. Particularly, the present disclosure relates to the medicament carrier advancing mechanism for a medicament dispenser. More particularly, the present disclosure relates to mechanism of actuation for a medicament dispenser.

In one of the embodiments, a medicament dispenser for dispensing medicament comprising: a) an assembly comprising: i) at least one peelable medicament carrier carrying multiple distinct medicament portions; ii) a medicament carrier advancing mechanism; iii) a flow channel; and b) a hinged cover for the flow channel.

In an embodiment, a medicament dispenser for dispensing medicament comprising: a) an assembly comprising: i) at least one peelable medicament carrier carrying multiple distinct medicament portions; ii) a medicament carrier advancing mechanism; iii) a flow channel; iv) a driver gear; and b) a hinged cover for the flow channel.

In another embodiment, a medicament dispenser for dispensing medicament comprising: a) an assembly comprising: i) at least one peelable medicament carrier carrying multiple distinct medicament portions; ii) a medicament carrier advancing mechanism; iii) a flow channel; iv) a driver gear; v) an interim driver gear; and b) a hinged cover for the flow channel.

In some embodiments, medicament comprises one or more active pharmaceutical ingredients (APIs) and optionally, one or more pharmaceutically acceptable carriers. The active pharmaceutical ingredients and pharmaceutically acceptable carriers may be present in micronized, non-micronized form or mixtures thereof.

One or more active pharmaceutical ingredients (APIs) that can be used in the inventions selected from, analgesics, e.g., codeine, dihydromorphine, ergotamine, fentanyl or morphine; anginal preparations, e.g., diltiazem; antiallergics, e.g., cromoglycate (e.g. as the sodium salt), ketotifen or nedocromil (e.g. as the sodium salt); antiinfectives e.g., cephalosporins, penicillins, streptomycin, sulphonamides, tetracyclines and pentamidine; antihistamines, e.g., methapyrilene; anti-fungals, e.g. Pulmazole and voriconazole; anti-inflammatories, e.g., beclomethasone (e.g. as the dipropionate ester), fluticasone (e.g. as the propionate or furoate ester), flunisolide, budesonide, rofleponide, mometasone e.g. as the furoate ester), ciclesonide, triamcinolone (e.g. as the acetonide) or 6α,9α-difluoro-11β-hydroxy-16α-methyl-3-oxo-17α-propionyloxy-androsta-1,4-diene-17β-carbothioic acid S-(2-oxo-tetrahydro-furan-3-yl) ester; antitussives, e.g., noscapine; bronchodilators, e.g., albuterol (e.g. as free base or sulphate), salmeterol (e.g. as xinafoate), ephedrine, adrenaline, fenoterol (e.g. as hydrobromide), formoterol (e.g. as fumarate), isoprenaline, metaproterenol, phenylephrine, phenylpropanolamine, pirbuterol (e.g. as acetate), reproterol (e.g. as hydrochloride), rimiterol, terbutaline (e.g. as sulphate), isoetharine, tulobuterol or 4-hydroxy-7-[2-[[2-[[3-(2-phenylethoxy)propyl]sulfonyl]ethyl]amino]ethyl-2(3H)-benzothiazolone; adenosine 2a agonists, e.g. 2R,3R,4S,5R)-2-[6-Amino-2-(1S-hydroxymethyl-2-phenyl-ethylamino)-purin-9-yl]-5-(2- ethyl-2H-tetrazol-5-yl)-tetrahydrofuran-3,4-diol (e.g. as maleate); α4 integrin inhibitors e.g. (2S)-3-[4-(([4-(aminocarbonyl)-1-piperidinyl]carbonyl)oxy)phenyl]-2-[((2S-)-4- methyl-2-([2-(2-methylphenoxy)acetyl]amino)pentanoyl)amino]propanoic acid (e.g. as free acid or potassium salt), diuretics, e.g., amiloride; anticholinergics, e.g., ipratropium (e.g. as bromide), tiotropium, umeclidinium, glycopyrronium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagon; vaccines, diagnostics, and gene therapies. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament.

In some embodiments, the medicament may in aspects, be a mono-therapy (i.e. single active medicament containing) product or it may be a combination therapy (i.e. plural active medicaments containing) product. Suitable medicaments or medicament components of a combination therapy product are typically selected from the group consisting of anti-inflammatory agents (for example a corticosteroid particularly inhaled corticosteroid (ICS) or an NSAID), anticholinergic agents (for example, an M₁, M₂, M₁/M₂ or M₃ receptor antagonist particularly long-acting muscarinic antagonist (LAMA), other β₂-adrenoreceptor agonists particularly long-acting β₂-agonist (LABA), anti-infective agents (e.g. an antibiotic or an antiviral), and antihistamines. All suitable combinations are envisaged. The present invention also provides combination therapy product typically double or triple LAMA, LABA, ICS combinations.

Suitable anti-inflammatory agents include corticosteroids and NSAIDs. Suitable corticosteroids are those oral and inhaled corticosteroids and their pro-drugs which have anti-inflammatory activity. Examples include methyl prednisolone, prednisolone, dexamethasone, fluticasone propionate, fluticasone furoate, 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester, beclomethasone esters (e.g. the 17-propionate ester or the 17,21-dipropionate ester), budesonide, flunisolide, mometasone esters (e.g. the furoate ester), triamcinolone acetonide, rofleponide, ciclesonide, butixocort propionate, RPR-106541, and ST-126. Preferred corticosteroids include fluticasone propionate, fluticasone furoate 6α,9α-difluoro-11β-hydroxy-16α-methyl-17α-[(-4-methyl-1,3- thiazole-5-carbonyl)oxy]-3-oxo-androsta-1,4-diene-17βcarbothioic acid S-fluoromethyl ester and 6α,9α-difluoro-17α-[(2- furanylcarbonyl)oxy]-11β-hydroxy-16α-methyl-3-oxo-androsta-1,4-diene-17β-carbothioic acid S-fluoromethyl ester, more preferably 6α,9α-difluoro-17α-[(2-furanylcarbonyl)oxy]-11β-hydroxy-16α-methyl-3-oxo-androsta-1,4-diene- 17β-carbothioic acid S-fluoromethyl ester.

Suitable NSAIDs include cromolyns (e.g. sodium cromoglycate, nedocromil sodium), phosphodiesterase (PDE) inhibitors (e.g. theophylline, PDE4 inhibitors or mixed PDE3/PDE4 inhibitors), leukotriene antagonists, inhibitors of leukotriene synthesis, iNOS inhibitors, tryptase and elastase inhibitors, β₂integrin antagonists and adenosine receptor agonists or antagonists (e.g. adenosine 2a agonists), cytokine antagonists (e.g. chemokine antagonists) or inhibitors of cytokine synthesis. Suitable other β₂-adrenoreceptor agonists include salmeterol (e.g. as the xinafoate), salbutamol (e.g. as the sulphate or the free base), formoterol (e.g. as the fumarate), vilanterol (e.g. as the trifenatate), fenoterol or terbutaline and salts thereof.

Suitable phosphodiesterase 4 (PDE4) inhibitors include compounds that are known to inhibit the PDE4 enzyme or which are discovered to act as a PDE4 inhibitor, and which are only PDE4 inhibitors, not compounds which inhibit other members of the PDE family as well as PDE4. Generally it is preferred to use a PDE4 inhibitor which has an IC₅₀ ratio of about 0.1 or greater as regards the IC₅₀ for the PDE4 catalytic form which binds rolipram with a high affinity divided by the IC₅₀ for the form which binds rolipram with a low affinity. For the purposes of this disclosure, the cAMP catalytic site which binds R and S rolipram with a low affinity is denominated the “low affinity” binding site (LPDE 4) and the other form of this catalytic site which binds rolipram with a high affinity is denominated the “high affinity” binding site (HPDE 4). This term “HPDE4” should not be confused with the term “hPDE4” which is used to denote human PDE4.

A method for determining IC₅₀s ratios is set out in U.S. Pat. No. 5,998,428 which is incorporated herein in full by reference as though set out herein. See also PCT application WO 00/51599 for another description of said assay.

Suitable PDE4 inhibitors include those compounds which have a salutary therapeutic ratio, i.e., compounds which preferentially inhibit cAMP catalytic activity where the enzyme is in the form that binds rolipram with a low affinity, thereby reducing the side effects which apparently are linked to inhibiting the form which binds rolipram with a high affinity. Another way to state this is that the preferred compounds will have an IC₅₀ ratio of about 0.1 or greater as regards the IC₅₀ for the PDE4 catalytic form which binds rolipram with a high affinity divided by the IC₅₀ for the form which binds rolipram with a low affinity.

A further refinement of this standard is that of one wherein the PDE4 inhibitor has an IC50 ratio of about 0.1 or greater; said ratio is the ratio of the IC₅₀ value for competing with the binding of 1 nM of [³H]R-rolipram to a form of PDE4 which binds rolipram with a high affinity over the IC₅₀ value for inhibiting the PDE4 catalytic activity of a form which binds rolipram with a low affinity using 1 muM[³H]-cAMP as the substrate.

Most suitable are those PDE4 inhibitors which have an IC₅₀ ratio of greater than 0.5, and particularly those compounds having a ratio of greater than 1.0. Preferred compounds are cis 4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol]; these are examples of compounds which bind preferentially to the low affinity binding site and which have an IC₅₀ ratio of 0.1 or greater.

Other suitable medicament compounds include: cis-4-cyano-4-[3-(cyclopentyloxy)-4-methoxyphenyl]cyclohexane-1-carboxylic acid (also known as cilomalast) disclosed in U.S. Pat. No. 5,552,438 and its salts, esters, pro-drugs or physical forms; AWD-12-281 from elbion (Hofgen, N. et al. 15th EFMC Int Symp Med Chem (September 6-10, Edinburgh) 1998, Abst P. 98; CAS reference No. 247584020-9); a 9-benzyladenine derivative nominated NCS-613 (INSERM); D-4418 from Chiroscience and Schering-Plough; a benzodiazepine PDE4 inhibitor identified as CI-1018 (PD-168787) and attributed to Pfizer; a benzodioxole derivative disclosed by Kyowa Hakko in WO99/16766; K-34 from Kyowa Hakko; V-11294A from Napp (Landells, L. J. et al. Eur Resp J [Annu Cong Eur Resp Soc (September 19-23, Geneva) 1998] 1998, 12 (Suppl. 28): Abst P2393); roflumilast (CAS reference No 162401-32-3) and a pthalazinone (WO99/47505, the disclosure of which is hereby incorporated by reference) from Byk-Gulden; Pumafentrine, (−)-p-[(4aR*,10bS*)-9-ethoxy-1,2,3,4,4a,10b-hexahydro-methoxy-8-methoxy-2-methylbenzo[c][1,6]naphthyridin-6-yl]- N,N-diisopropylbenzamide which is a mixed PDE3/PDE4 inhibitor which has been prepared and published on by Byk-Gulden, now Altana; arofylline under development by Almirall-Prodesfarma; VM554/UM565 from Vernalis; or T-440 (Tanabe Seiyaku; Fuji, K. et al. J Pharmacol Exp Ther, 1998, 284(1): 162), and T2585.

Suitable anticholinergic agents are those compounds that act as antagonists at the muscarinic receptor, in particular those compounds, which are antagonists of the M1 and M2 receptors. Exemplary compounds include the alkaloids of the belladonna plants as illustrated by the likes of atropine, scopolamine, homatropine, hyoscyamine; these compounds are normally administered as a salt, being tertiary amines.

Particularly suitable anticholinergics include ipratropium (e.g. as the bromide), sold under the name Atrovent, umeclidinium (e.g. as the bromide), glycopyrronium (e.g. as the bromide), oxitropium (e.g. as the bromide) and tiotropium (e.g. as the bromide) (CAS-139404-48-1). Also of interest are: methantheline (CAS-53-46-3), propantheline bromide (CAS-50-34-9), anisotropine methyl bromide or Valpin 50 (CAS-80-50-2), clidinium bromide (Quarzan, CAS-3485-62-9), copyrrolate (Robinul), isopropamide iodide (CAS-71-81-8), mepenzolate bromide (U.S. Pat. No. 2,918,408), tridihexethyl chloride (Pathilone, CAS-4310-35-4), and hexocyclium methylsulfate (Tral, CAS-115-63-9). See also cyclopentolate hydrochloride (CAS-5870-29-1), tropicamide (CAS-1508-75-4), trihexyphenidyl hydrochloride (CAS-144-11-6), pirenzepine (CAS-29868-97-1), telenzepine (CAS-80880-90-9), AF-DX 116, or methoctramine, and the compounds disclosed in WO01/04118.

Suitable antihistamines (also referred to as H₁-receptor antagonists) include any one or more of the numerous antagonists known which inhibit H₁-receptors, and are safe for human use. All are reversible, competitive inhibitors of the interaction of histamine with H1-receptors. Examples include ethanolamines, ethylenediamines, and alkylamines. In addition, other first generation antihistamines include those which can be characterized as based on piperizine and phenothiazines. Second generation antagonists, which are non-sedating, have a similar structure-activity relationship in that they retain the core ethylene group (the alkylamines) or mimic the tertiary amine group with piperizine or piperidine. Exemplary antagonists are as follows: Ethanolamines: carbinoxamine maleate, clemastine fumarate, diphenylhydramine hydrochloride, and dimenhydrinate. Ethylenediamines: pyrilamine amleate, tripelennamine HCl, and tripelennamine citrate. Alkylamines: chlropheniramine and its salts such as the maleate salt, and acrivastine. Piperazines: hydroxyzine HCl, hydroxyzine pamoate, cyclizine HCl, cyclizine lactate, meclizine HCl, and cetirizine HCl. Piperidines: Astemizole, levocabastine HCl, loratadine or its descarboethoxy analogue, and terfenadine and fexofenadine hydrochloride or another pharmaceutically acceptable salt.

Azelastine hydrochloride is yet another H₁receptor antagonist which may be used in combination with a PDE4 inhibitor. Particularly suitable anti-histamines include methapyrilene and loratadine. In respect of combination products, co-formulation compatibility is generally determined on an experimental basis by known methods and may depend on chosen type of medicament dispenser action.

The medicament components of a combination product are suitably selected from the group consisting of anti-inflammatory agents (for example a corticosteroid or an NSAID), anticholinergic agents (for example, an M₁, M₂, M₁/M₂ or M₃ receptor antagonist), other β₂-adrenoreceptor agonists, anti-infective agents (e.g. an antibiotic or an antiviral), and antihistamines. All suitable combinations are envisaged.

Suitably, the co-formulation compatible components comprise a β₂-adrenoreceptor agonist and a corticosteroid; and the co-formulation incompatible component comprises a PDE-4 inhibitor, an anti-cholinergic or a mixture thereof. The β₂-adrenoreceptor agonists may for example be salbutamol (e.g., as the free base or the sulphate salt) or salmeterol (e.g., as the xinafoate salt) or formoterol (e.g., as the fumarate salt). The corticosteroid may for example, be a beclomethasone ester (e.g., the dipropionate) or a fluticasone ester (e.g., the propionate) or budesonide.

In one example, the co-formulation compatible components comprise fluticasone propionate and salmeterol, or a salt thereof (particularly the xinafoate salt) and the co-formulation incompatible component comprises a PDE-4 inhibitor, an anti-cholinergic (e.g. ipratropium bromide or tiotropium bromide) or a mixture thereof.

In another example, the co-formulation compatible components comprise budesonide and formoterol (e.g. as the fumarate salt) and the co-formulation incompatible component comprises a PDE-4 inhibitor, an anti-cholinergic (e.g. ipratropium bromide or tiotropium bromide) or a mixture thereof.

Medicament dispensers, as disclosed herein, have a variety of structural configurations and can be used for dispensing powders, tablets, capsules, pellets or pucks, or mixtures thereof for nasal, pulmonary or oral administration. In particular, the medicament dispensers can be used to dispense a powder comprising one or more active pharmaceutical ingredient(s)(API) and optionally, one or more micronized or non-micronized pharmaceutically acceptable carrier(s) and/or excipient(s), wherein the dispensing mechanism comprises of three steps to operate the medicament dispenser: Open, Dispense and Close.

Generally, powdered medicament particles suitable for delivery to the bronchial or alveolar region of the lung have an aerodynamic diameter of less than 10 micrometers, preferably less than 6 micrometers. Other sized particles may be used if delivery to other portions of the respiratory tract is desired, such as the nasal cavity, mouth or throat. The medicament may be delivered as pure drug, but more appropriately, it is preferred that medicaments are delivered together with one or more excipients or carriers which are suitable for inhalation. Suitable excipients or carriers include organic excipients such as polysaccharides (i.e. starch, cellulose and the like), lactose, trehalose, leucine, trileucine, pentaleucine and other polyleucines, glucose, mannitol, amino acids, magnesium stearate and maltodextrins, surfactants such as 1,2-Distearoyl-sn-glycero-3-phosphocholine (DSPC), 1,2-Distearoyl-sn-glycero-3-phosphoethanolamine (DSPE), perfluorocarbon such as perflubron (perfluorooctyl bromide) and inorganic excipients such as calcium carbonate, calcium chloride, magnesium chloride or sodium chloride. In preferred embodiment, carrier or excipient is lactose.

Particles of powdered medicament and/or excipient may be produced by conventional techniques, for example by micronisation, milling or sieving. Additionally, medicament and/or excipient powders may be engineered with particular densities, size ranges, or characteristics. Particles may comprise active agents, surfactants, wall forming materials, or other components considered desirable by those of ordinary skill.

The medicament dispensers described herein can be used for the treatment and prophylaxis of respiratory diseases, including, but not limited to asthma, chronic obstructive pulmonary disease (COPD), bronchitis lung infections, interstitial pulmonary fibrosis (IPF), pulmonary arterial hypertension (PAH), cystic fibrosis, Non-CF bronchiectasis or lung transplant.

The medicament dispenser can be a hand-held dispenser and can be operated by a single hand.

The medicament dispenser can be of circular form, non-circular form, an elongate form, an elliptical form, an ovular form, an oblong form, a triangular form, a quadrilateral form, including, but not limited to square form, rectangular form and various trapezium forms, an hour glass form, or other shapes.

The medicament dispenser can include a flat surface or a resting surface. At least a portion of the medicament dispenser can be shaped for ease of grip by the user.

The medicament dispenser can be an inhalation device, in particular a dry powder inhaler (DPI).

The medicament dispenser can include an assembly or a housing.

The assembly can include a base cover and a top cover for enclosing a medicament carrier, a medicament carrier advancing mechanism, inner housing assembly, inner housing support, dose indicator or dose counter. The top cover can lock with the base cover to form a flow channel (or opening). The top cover can permanently lock with the base cover through permanent bonding methods, including, but not limited to ultrasonic welding and ultraviolet cured adhesives and the like.

The flow channel can be in the form of a mouthpiece for oral use. The flow channel can be in the form of a nozzle for insertion into the nasal cavity of a patient, or the flow channel can be in the form of exit channel if the medicament is in solid form such as tablets or capsules.

The flow channel can be a single outlet, which communicates with all of the distinct medicament on their release. Communication can be for example, via a common air channeling means (e.g. formed as an air-pipe or common manifold). The flow channel and/or channeling device can be shaped to encourage mixing of drug as a result of the airflow created by inhalation by the patient.

The term ‘mouthpiece’ is used to mean an element through which the patient may inhale. In one aspect, the inhalation is by oral means with the patient placing the mouthpiece in its mouth. In an alternative aspect, the mouthpiece comprises a nozzle for insertion into the nasal cavity of a patient.

The mouthpiece can snap on the assembly. The mouthpiece can further include a mouthpiece interface opening. The mouthpiece interface opening and flow channel can allow the distinct medicament portions to be dispensed to the user. The mouthpiece can be in the form of an extended mouthpiece to assist with de-agglomeration of the medicament and ease of delivery in the oral cavity.

The assembly can be of circular form, non-circular form, an elongate form, an elliptical form, an ovular form, an oblong form, a triangular form, a quadrilateral form, including, but not limited to square form, rectangular form and various trapezium forms, an hour glass form, or other shapes. The assembly can include a flat surface or a resting surface. At least a portion of the assembly can be shaped for ease of grip by the user.

The assembly can include at least one medicament carrier carrying multiple distinct medicament portions.

The assembly can include a single or plural medicament carriers. The plural elongate form medicament carriers may be provided in any suitable configuration. One suitable configuration is the ‘side-by-side’ configuration, in which for example, two carriers (e.g. two coiled blister strips) are arranged to lie in sideways alignment with each other. Another suitable configuration is the ‘double-decker’ configuration, in which for example, two carriers (e.g. two coiled blister strips sharing the same coiling axis) are arranged to lay one on top of each other in the dispenser. The plural medicament carriers can be a first medicament carrier and a second medicament carrier. The plural medicament carriers can be separated by a deck separator or deck separators. The deck separator can be used to allow the independent movement of the medicament carriers with no contact between medicament carriers and no friction between medicament carriers due to mutual contact. The deck separators can be locked in the inner housing assembly by locking heads.

The multiple distinct dose portions can be provided to each carrier in uniform series. In particular, the spacing (i.e. pitch) between each dose portion is uniform throughout the series. In other aspects however, the spacing (i.e. pitch) may vary throughout the series (i.e. be non-uniform). In one aspect, the spacing (i.e. pitch) between each dose portion is equivalent for each carrier of the dispenser. That is to say, each medicament carrier is equivalently pitched. In other aspects, the spacing between each dose portion is non-equivalent for each carrier of the dispenser. Such variation of the spacing (i.e. pitch) from carrier to carrier can be used to enable flexibility in (combination) dosage patterns. In one particular example, the spacing (i.e. pitch) of a first carrier is arranged to be half that of a second carrier. This arrangement is beneficially employed where the dose interval (i.e. time between doses) of the medicament carried by the first carrier is twice that of the medicament carrier by the second carrier (e.g. in a twice daily/once daily type dosage regime).

The medicament carrier carrying multiple distinct medicament portions can be in the form of a strip or tap. For example, the medicament carrier can be a reel of blisters containing multiple distinct blister pockets (cavities) such as a blister pack or a blister strip. The medicament carrier can be a blister pack in a laminate form. Access to the medicament(s) comprised within the blister pockets of reel of blisters is by any suitable access means including piercing, tearing or peeling apart the relevant blister pockets. The medicament carrier can be wound around a post of the inner housing assembly. One end of the medicament carrier can be in the form of a sealed loop that can be placed in a blister post of a winder ratchet. The other end of the medicament carrier can be placed in a slit of a wrapping wheel.

The reel of blisters can be in the form of a peelable blister strip which includes blister pockets wherein each of the blister pockets has one dose of the medicament. As another example, each blister pocket comprises or forms two or more separate chambers or compartments, for example two compartments. The compartments are formed by a partition wall dividing the blister pocket. The peelable blister strip comprises a base sheet in which blisters are formed to define pockets therein for containing distinct medicament dose portions and a lid sheet or lamination which is hermetically sealed to the base sheet except in the region of the blisters in such a manner that the lid sheet and the base sheet can be peeled apart. The blister pocket having compartments may be covered by common lid sheet. The lamination can be peeled via a constant tension torque applied by the winder assembly. The lamination winds around the winder assembly. The base sheet advances via the wrapping wheel.

The blister pocket from the reel of blisters can include medicament comprising one or more active pharmaceutical ingredient(s). For example, the blister pocket can include medicament comprising a single active pharmaceutical ingredient. In other examples, the blister pocket can include medicament comprising two, three, four, or more active pharmaceutical ingredients. For example, the blister pocket can include medicament comprising two or three active pharmaceutical ingredients.

The inner housing assembly can house the medicament carrier. The inner housing assembly can include a mesh or meshes that allow de-agglomeration of the medicament. During dosing operation, the cuvette on the blister cuvette is aligned with the mesh which can allow the medicament to be dispensed through the flow channel and mouthpiece to the user. The inner housing assembly can be locked and placed in the dispenser on the inner housing support.

The medicament dispenser can include a cover. The cover can be a hinged cover and/or attached to the assembly by a hinge. By hinging the cover to the assembly in that way the user can simply and conveniently open the cover by gripping the medicament dispenser with one hand, for example by placing a thumb on the cover and fingers on the back of the assembly. This construction avoids many of the real difficulties that some users experience when trying to open commercially available inhalers. This is especially true for users who are old, fragile, disabled or for some other reason have impaired dexterity that makes it difficult or perhaps even impossible for them to grip certain inhalers or to use inhalers that require a swivel or some other twisting action to be opened. The hinged cover can be pivotally attached to a side of the assembly. The cover and the assembly may not have a common axis. For example, an axis about which the cover pivots can be displaced or radially outward of a central axis extending through the top cover and the base cover of the assembly. The axis of rotation of the cover can be outside the plane of the assembly. The cover may be configured such that it does not move from an initial position to a dispense position over a rotational path. The cover may be configured such that it does not slide along the surface of the assembly. The medicament dispenser can include side axis driven hinged cover.

The hinged cover can have a hinged end pivotally attached to the assembly and a free end opposite the hinged end. Rotation of the cover about the hinged end rotates the free end of the cover to a position outside of a footprint of the assembly.

The hinged cover can include a mounting hole, a pivot hole and a boss.

The hinged cover can be adapted to couple with the driver gear and the medicament carrier advancing mechanism.

The hinged cover can be movably mounted to the medicament dispenser for sequential movement from an initial position, in which the flow channel is covered, to a dispense position, in which the flow channel is uncovered. The flow channel can be fully revealed when the hinged cover is in a dispense position. When the cover is in the dispense position, the entire cover may remain entirely above a transverse centerline of the assembly.

The movement of the hinged cover from the initial position can rotate the driver gear and actuate the medicament carrier advancing mechanism.

The movement of the hinged cover from the initial position can rotate the driver gear and the interim driver gear and actuate the medicament carrier advancing mechanism. The hinged cover can begin to rotate the driver gear upon any rotation of the hinged cover, such that dispensation begins without any primed or intermediate position. The hinged cover and the driver gear can rotate synchronously. The hinged cover actuates the medicament carrier advancing mechanism without any sequential actuation steps, such as actuating a lever.

The movement of the hinged cover from the initial position can result in actuation of the medicament carrier advancing mechanism.

The dispensing of the medicament can provide continuous movement of the hinged cover from the initial position to the dispense position of the hinged cover without any intermediate position.

The dispensing of the medicament can provide continuous movement of the hinged cover from the initial position to rotate the driver gear and actuate the medicament carrier advancing mechanism.

The dispensing of the medicament can provide continuous movement of the hinged cover from the initial position to rotate the driver gear and the interim driver gear and actuate the medicament carrier advancing mechanism.

The dispensing of the medicament can provide continuous movement of the hinged cover from the initial position to actuate the medicament carrier advancing mechanism

The term “initial position” as used herein is interchangeable with “initial state”, “rest position”, “rest state” or “closed position” and refers to the position of the hinged cover wherein the hinged cover covers the flow channel and/or mouthpiece, and user cannot access the medicament.

The term “dispense position” as used herein is interchangeable with “open position” and refers to the position of the hinged cover wherein the hinged cover does not cover the flow channel and/or mouthpiece, and user can access the medicament.

The dispensing of the medicament may not provide any idle movement of the hinged cover.

The hinged cover can be of circular form, non-circular form, an elongate form, an elliptical form, an ovular form, an oblong form, a triangular form, a quadrilateral form, including, but not limited to square form, rectangular form and various trapezium forms, an hour glass form, or other shapes. The hinged cover can provide a flat surface or a resting surface.

At least a portion of the hinged cover can be shaped for ease of grip by the user. The hinged cover can include dots or waves gripping to provide for an efficient gripping.

The medicament dispenser can be prepared for dosing by achieving an angle of movement of the hinged cover between about 37° to about 135° relative to the rest position of the hinged cover. The angle of movement of the hinged cover can be between about 37° to about 180° relative to the rest position of the hinged cover. The angle of movement of the hinged cover can be between about 120° to about 180° relative to the rest position of the hinged cover. The angle of movement of the hinged cover between about 120° to about 135° relative to the rest position of the hinged cover. The angle of movement of the hinged cover between about 135° to about 180° relative to the rest position of the hinged cover. The angle of movement of the hinged cover between about 160° to about 180° relative to the rest position of the hinged cover.

The medicament dispenser can include at least one locking mechanism, enabling the dispenser to remain locked in at least one position in which the dispenser is ready for inhalation and/or the cover is in the closed position. The locking mechanism can be used for preventing unintended and accidental opening of the cover. The locking mechanism can be used for preventing further rotation of the cover once it is completely opened (actuated). The locking mechanism can be used for indicating that the medicament dispenser is ready for dispensing the dose. The locking mechanism can be used for providing an audible and haptic or kinesthetic feedback to the user indicating complete opening and complete closing of the cover.

The locking mechanism can include a first ridge which is disposed on the top cover, and a second ridge which is disposed on the base cover. The locking mechanism can also include ridges which can be located symmetrically on both the surfaces of the hinged cover. The first ridge on the top cover and the second ridge on the base cover provide for a locking mechanism via corresponding ridges on the hinged cover. The ridges on the hinged cover can snap over the first ridge on the top cover and the second ridge on the base cover. The locking mechanism can include a third ridge which is disposed to a side of the top cover. The edge on the hinged cover can snap over the third ridge.

The locking mechanism can include a locking stud which is disposed on the base cover. The locking mechanism can also include a locking notch, a locking space and a locking groove. The locking groove can be guided by the locking stud. The locking notch causes the locking stud to snap within the locking space both during opening and closing of the cover.

The locking mechanism can include a locking pin which is located on the inner surface of the cover. The locking mechanism can also include a locking vent located on the mouthpiece. The locking pin can lock with the locking vent. The locking mechanism between the locking vent and the locking pin provides resistance towards opening and closing of the cover.

The locking mechanism can include a projection which is located on the inner surface of the cover that mates with the opening of the mouthpiece. The locking mechanism between the projection and the opening of the mouthpiece provides resistance towards opening and closing of the cover.

The assembly can include a driver gear. The driver gear can include a bearing, teeth, a flange and a shaft. The driver gear can be driven by the cover via the flange. The driver gear can be used to drive the cuvette ratchet.

The assembly can include an interim driver gear. The interim driver gear can include teeth. The driver gear can be connected to the interim driver gear which is connected to the cuvette ratchet. The interim driver gear can be used to drive the cuvette ratchet. The interim driver gear can allow the hinged cover to move about 135° to about 180° relative to the rest position of the hinged cover.

The medicament carrier advancing mechanism for actuating the distinct medicament portions carried by the medicament carrier can include a cuvette ratchet, a blister cuvette, a winder assembly, an interim gear and a wrapping wheel. The blister cuvette can include a central hole, cuvettes, spur gears, contacting surfaces and non-contacting surfaces. The blister cuvette can be driven by the ratcheting mechanism of the cuvette ratchet. The cuvettes on the blister cuvette can locate the individual blister pocket on the medicament carriers. The spur gears of the blister cuvette can mate with the interim gear and the winder gear. The cuvette ratchet can include a central boss, ratchets and spur gears. The cuvette ratchet can ride over the central boss of the base cover. The cuvette ratchet can be guided on the central boss by a bearing. The ratchets located diametrically opposite on the cuvette ratchet and rest on the contacting surface on the blister cuvette. The ratchets drive the blister cuvette in one direction only through the contacting surface. The ratcheting mechanism of the cuvette ratchet prevents the rotation of the blister cuvette in the opposite direction. The ratchets ride over the non-contacting surface of the blister cuvette without rotating the blister cuvette. The winder assembly can include a winder wheel, a winder ratchet and a winder gear. The winder wheel can include a winding surface, a recess opening and a winding post. The winder wheel can include a ridge for separating the laminations of the plural medicament carriers during the winding process. The winder ratchet can include a ratchet, locking pins, a blister post and a flange. The winder gear can include inner gears and outer gears. The wrapping wheel can include a slit, a gear and spur gears.

The actuation of the medicament dispenser can rotate the cuvette ratchet and the blister cuvette synchronously and thereby advances the medicament carrier to align with the flow channel for dispensing the medicament.

The base cover can hold and encase all the moving components within the assembly. The base cover can include an end boss, a central boss, studs, a locating ring, a bearing surface, an end pin and a window through which the dose count may be viewed.

The top cover can include a mouthpiece, a mounting hole, air inlet vent holes and mouthpiece vent holes. The air inlet vent holes can be located discretely on the top cover to provide for an air inlet through the medicament dispenser. The mouthpiece vent holes can be provided for by-pass air outlet. The air can enter the device through the air inlet vent holes and passes through the intermittent spaces between the device components and the inner housing assembly. The air can exit the device through the mouthpiece and through the mouthpiece vent holes. For example, the air can exit the device at an angle about 80° to about 100° relative to the angle of air entrance. As another example, the air can exit the device at an angle about 90° relative to the angle of air entrance. The flow of air contributes to the aerosolization of the contents of the blister cavity and thereby emptying of the contents of the blister cavities via the mouthpiece.

The medicament dispenser can include a dose counting or an indicating mechanism for counting or indicating number of doses released from the dispenser.

The medicament dispenser can include an actuation or dose counter for counting the number of actuations or releases of dose from the medicament dispenser. The dose counter may count the number of doses left to be taken or the number of doses taken. The dose counter may be mechanical (e.g., non-electronic) or non-mechanical (e.g., electronic, digital) in form.

The medicament dispenser can include a mechanism of actuation wherein the mechanism of actuation also provides for a mechanism for increase in dose counter reading during dispensation of the medicament.

The medicament dispenser can include a mechanism of actuation wherein the movement of the mechanism of actuation also provides for a mechanism for increase in a mechanical (i.e. non-electronic) and a non-mechanical (e.g., electronic, digital) dose counter reading during dispensation of the medicament.

The dose counter can be a part of assembly such as a mechanical dose ring. The dose counter can be located in a compartment of the hinged cover having dedicated space to accommodate said dose counter.

The dose counting or an indicating mechanism can be in the form of a dose indicator assembly. The dose indicator assembly can include a dose indicator driving gear, a dose indicator units wheel, a dose indicator transfer wheel and a dose indicator tens wheel.

The dose indicator assembly can include the dose indicator units wheel arranged to rotate about a first axis of rotation. The dose indicator units wheel may for example, take the form of a disc or a ring.

The dose indicator units wheel can include count indicia and a set of teeth arranged annularly, preferably circumferentially thereon. The teeth can be therefore arranged in annular fashion at or about the circumference of the dose indicator units wheel. The dose indicator units wheel displays the units place of the dose. The dose indicator units wheel can further include inner gears.

The dose indicator assembly can further include the dose indicator tens wheel arranged to rotate about a second axis of rotation.

The dose indicator tens wheel can include count indicia and a set of teeth arranged annularly, preferably circumferentially thereon. The teeth can be therefore arranged in annular fashion at or about the circumference of the dose indicator tens wheel. The dose indicator tens wheel can further include a dose indicator stopper.

The dose indicator transfer wheel can include spur gears arranged annularly thereon and in meshed relationship with the set of teeth of the dose indicator tens wheel. The dose indicator transfer wheel can further include a set of teeth arranged annularly thereon.

The inner gears of the dose indicator units wheel can be arranged for intermittent meshing with the teeth of the dose indicator transfer wheel.

The medicament dispenser can include a dose indicator assembly for use with a medicament dispenser, said dose indicator assembly comprising a dose indicator units wheel arranged to rotate about a first axis of rotation, said dose indicator units wheel including a set of teeth arranged annularly thereon for drivable rotation of the dose indicator units wheel about said first axis of rotation; a dose indicator tens wheel arranged to rotate about a second axis of rotation, said dose indicator tens wheel including a set of teeth arranged annularly thereon; and a dose indicator transfer wheel arranged to rotate about a third axis of rotation offset from the first axis of rotation and the second axis of rotation, said dose indicator transfer wheel including a set of teeth arranged annularly thereon and in meshed relationship with the set of teeth of the dose indicator tens wheel such that rotary motion of the dose indicator transfer wheel results in rotary motion of the dose indicator tens wheel, wherein said dose indicator units wheel further includes inner gears arranged for intermittent meshing with spur gears of the dose indicator transfer wheel such that rotary motion of the dose indicator transfer wheel results from rotary motion of the dose indicator units wheel only when said intermittent meshing occurs.

The dose indicator can be a dose indicator wheel. The dose indicator wheel may for example, take the form of a disc or a ring. The dose indicator wheel can include count indicia and a set of teeth arranged annularly, preferably circumferentially thereon. The teeth can be therefore arranged in annular fashion at or about the circumference of the dose indicator wheel. In some embodiments, the dose indicator wheel can further include a dose indicator stopper.

The medicament dispenser can include a space for a power source (e.g., battery), electronic circuit and electronic measurement instruments. The power source (e.g., battery), electronic circuit and electronic measurement instruments can be located in a compartment of the hinged cover having dedicated space to accommodate said power source (e.g., battery), electronic circuit and electronic measurement instruments.

The medicament dispenser can include an electronic data management system. The electronic data management system can include input/output capability and comprises a memory for storage of data; a microprocessor for performing operations on said data; and a transmitter for transmitting a signal relating to the data or the outcome of an operation on the data. The electronic data management system may be integral with the body of the dispenser. Alternatively, the electronic data management system forms part of a base unit that is reversibly associable with the body.

The medicament dispenser can be made from the material of constructions comprises of acrylonitrile butadiene styrene (ABS), polycarbonate/acrylonitrile butadiene styrene terpolymer blend (PC/ABS), Polyoxymethylene (POM), nylon and silicone rubber.

In some embodiments, a medicament dispenser for dispensing medicament comprises: a) an assembly comprising: i) at least one peelable medicament carrier carrying multiple distinct medicament portions; ii) a medicament carrier advancing mechanism; iii) a flow channel; iv) a driver gear; and b) a hinged cover for the flow channel, wherein the hinged cover is adapted to couple with the driver gear and the medicament carrier advancing mechanism.

In some embodiments, a medicament dispenser for dispensing medicament comprises: a) an assembly comprising: i) at least one peelable medicament carrier carrying multiple distinct medicament portions; ii) a medicament carrier advancing mechanism; iii) a flow channel; iv) a driver gear; and b) a hinged cover for the flow channel, wherein the hinged cover being movably mounted to the dispenser for sequential movement from an initial position, in which said flow channel is covered, to a dispense position, in which said flow channel is uncovered.

In some embodiments, a medicament dispenser for dispensing medicament comprises: a) an assembly comprising: i) at least one peelable medicament carrier carrying multiple distinct medicament portions; ii) a medicament carrier advancing mechanism; iii) a flow channel; iv) a driver gear; and b) a hinged cover for said flow channel, wherein said hinged cover is adapted to couple with said medicament carrier advancing mechanism such that the movement of the hinged cover from an initial position rotates the driver gear to actuate the medicament carrier advancing mechanism.

In some embodiments, a medicament dispenser for dispensing medicament comprises: a) an assembly comprising: i) at least one peelable medicament carrier carrying multiple distinct medicament portions; ii) a mechanism of actuation for dispensing the distinct medicament portions; iii) a flow channel; iv) a driver gear; and b) a hinged cover for said flow channel, wherein the mechanism of actuation provides continuous movement of hinged cover without any idling position between an initial position and a dispense position.

In some embodiments, a medicament dispenser for dispensing medicament comprises: a) an assembly comprising: i) at least one peelable medicament carrier carrying multiple distinct medicament portions; ii) a medicament carrier advancing mechanism iii) a flow channel; iv) a driver gear; and b) a hinged cover for the flow channel, wherein the flow channel is covered by the hinged cover in an initial position, and fully revealed when the hinged cover is in a dispense position.

In some embodiments, a medicament dispenser for dispensing medicament comprises: a) an assembly comprising: i) at least one peelable medicament carrier carrying multiple distinct medicament portions; ii) a medicament carrier advancing mechanism iii) a flow channel; iv) a driver gear; and b) a hinged cover for the flow channel, wherein the flow channel is covered by the hinged cover in an initial position, and fully revealed when the hinged cover is in a dispense position.

In some embodiments, a medicament dispenser for dispensing medicament comprises: a) an assembly comprising: i) at least one peelable medicament carrier carrying multiple distinct medicament portions; ii) a mechanism of actuation for dispensing the distinct medicament portions; iii) a flow channel; iv) a mouthpiece iv) a driver gear; and b) a hinged cover for said mouthpiece comprises, wherein the mechanism of actuation provides continuous movement of the hinged cover such that there is no intermediate position for hinged cover between an initial position and a dispense position.

In some embodiments, a medicament dispenser for dispensing medicament comprises: a) an assembly comprising: i) at least one peelable medicament carrier carrying multiple distinct medicament portions; ii) a medicament carrier advancing mechanism for actuating the distinct medicament portions; iii) a flow channel; iv) a driver gear; and b) a hinged cover for said flow channel, wherein the coupling of the hinged cover with the medicament carrier advancing mechanism is adapted such the movement of the cover from an initial position to a dispense position results in actuation of dispensing mechanism without any idle movement of the hinged cover.

In some embodiments, a medicament dispenser for dispensing medicament comprises: a) an assembly comprising: i) at least one peelable medicament carrier carrying multiple distinct medicament portions; ii) a medicament carrier advancing mechanism; iii) a flow channel; iv) a driver gear; and b) a hinged cover for said flow channel, wherein the dispenser is an inhalation device.

In some embodiments, a medicament dispenser for dispensing medicament comprises: a) an assembly comprising: a medicament carrier; a flow channel; a medicament carrier advancing mechanism; a driver gear; and b) a hinged cover, wherein the hinged cover is movable form an at initial position, in which the hinged cover covers said flow channel, to a dispense position to uncover the flow channel, and the movement of the hinged cover coupled to said medicament carrier advancing mechanism by the driver gear that are arranged such that movement of the hinged cover from the initial position to the dispense position results in actuation of the medicament carrier advancing mechanism by the driver gear thereby advances the medicament carrier to align that with the flow channel, and through which a user can access the medicament from said flow channel, and movement of the hinged cover from the dispense position to the initial position results in rotation of the driver gear and without advancing the medicament carrier.

In some embodiments, a medicament dispenser for dispensing medicament comprises: a) an assembly comprising: i) at least one peelable medicament carrier carrying multiple distinct medicament portions; ii) a medicament carrier advancing mechanism; iii) a flow channel; iv) a driver gear; v) an interim driver gear; and b) a hinged cover for the flow channel, wherein the hinged cover being movably mounted to the dispenser for sequential movement from an initial position, in which said flow channel is covered, to a dispense position, in which said flow channel is uncovered.

In some embodiments, a medicament dispenser for dispensing medicament comprises: a) an assembly comprising: i) at least one peelable medicament carrier carrying multiple distinct medicament portions; ii) a medicament carrier advancing mechanism; iii) a flow channel; iv) a driver gear; v) an interim driver gear; and b) a hinged cover for said flow channel, wherein said hinged cover is adapted to couple with said medicament carrier advancing mechanism such that the movement of the hinged cover from an initial position rotates the driver gear and the interim driver gear to actuate the medicament carrier advancing mechanism.

In some embodiments, a medicament dispenser for dispensing medicament comprises: a) an assembly comprising: i) at least one peelable medicament carrier carrying multiple distinct medicament portions; ii) a mechanism of actuation for dispensing the distinct medicament portions; iii) a flow channel; iv) a driver gear; v) an interim driver gear; and b) a hinged cover for said flow channel, wherein the mechanism of actuation provides continuous movement of hinged cover without any idling position between an initial position and a dispense position.

In some embodiments, a medicament dispenser for dispensing medicament comprises: a) an assembly comprising: i) at least one peelable medicament carrier carrying multiple distinct medicament portions; ii) a medicament carrier advancing mechanism iii) a flow channel; iv) a driver gear; v) an interim driver gear; and b) a hinged cover for the flow channel, wherein the flow channel is covered by the hinged cover in an initial position, and fully revealed when the hinged cover is in a dispense position.

In some embodiments, a medicament dispenser for dispensing medicament comprises: a) an assembly comprising: i) at least one peelable medicament carrier carrying multiple distinct medicament portions; ii) a mechanism of actuation for dispensing the distinct medicament portions; iii) a flow channel; iv) a mouthpiece iv) a driver gear; v) an interim driver gear; and b) a hinged cover for said mouthpiece comprises, wherein the mechanism of actuation provides continuous movement of the hinged cover such that there is no intermediate position for hinged cover between an initial position and a dispense position.

In some embodiments, a medicament dispenser for dispensing medicament comprises: a) an assembly comprising: i) at least one peelable medicament carrier carrying multiple distinct medicament portions; ii) a medicament carrier advancing mechanism for actuating the distinct medicament portions; iii) a flow channel; iv) a driver gear; v) an interim driver gear; and b) a hinged cover for said flow channel, wherein the coupling of the hinged cover with the medicament carrier advancing mechanism is adapted such the movement of the cover from an initial position to a dispense position results in actuation of dispensing mechanism without any idle movement of the hinged cover.

It is intended that the scope of the present invention herein disclosed should not be limited by any particular embodiment described herein. While various embodiments of the present invention have been described above, it should be noted that they have been presented by way of example only, and not limitation. Numerous changes to the disclosed embodiments can be made in accordance with the disclosure herein without departing from the spirit or scope of the invention.

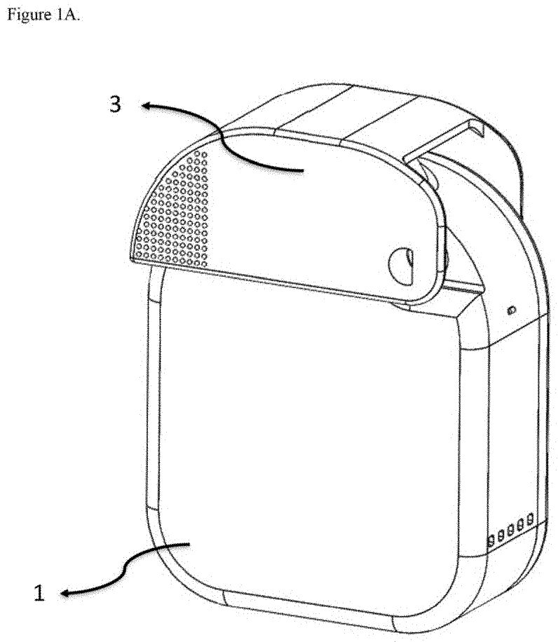

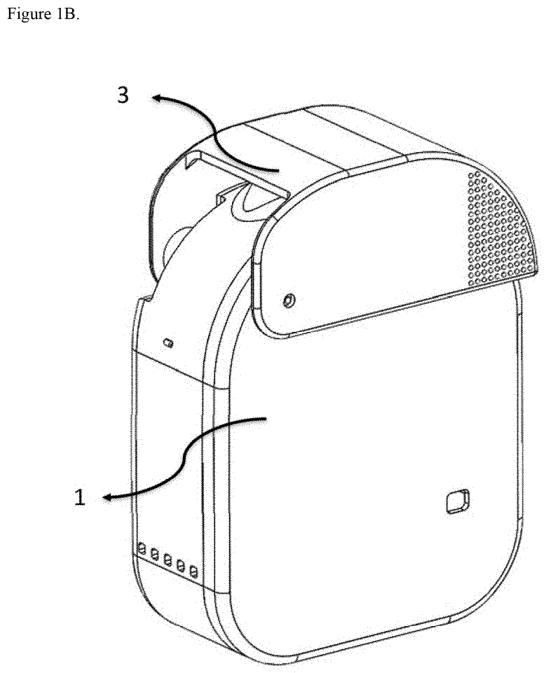

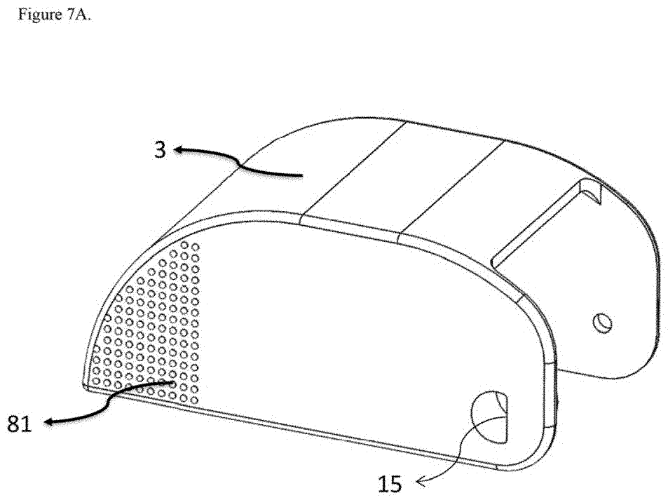

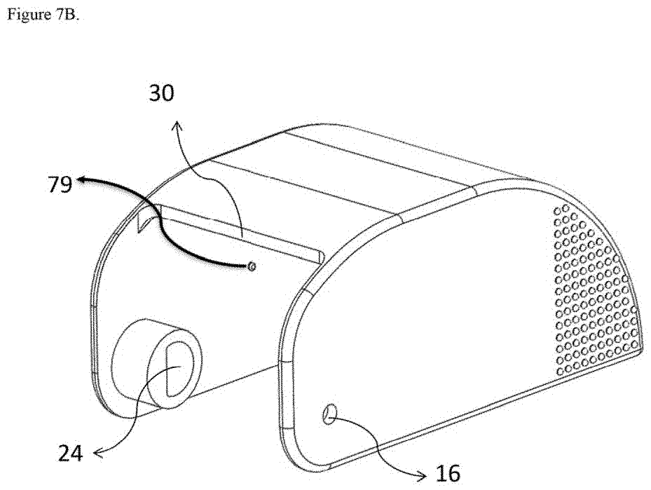

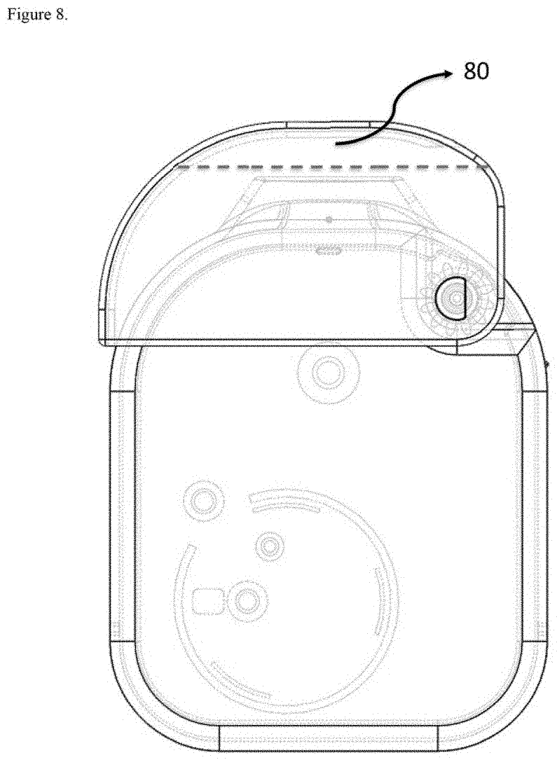

FIGS. 1A and 1B illustrate different views of the medicament dispenser according to one aspect of the disclosure. The medicament dispenser can include an assembly (1) which houses a reel of blisters (5) and the blister advancing mechanism. The assembly (1) can include the base cover (14) and the top cover (13) encasing all the moving components within the assembly. The blister advancing mechanism can be operated (actuated) via the hinged cover (3). The hinged cover (3) can be mechanically mounted to the assembly (1). The hinged cover (3) (see FIGS. 7A & 7B) can include the boss (24) to prevent the hinged cover (3) from getting disassembled from the assembly during operation of the medicament dispenser.

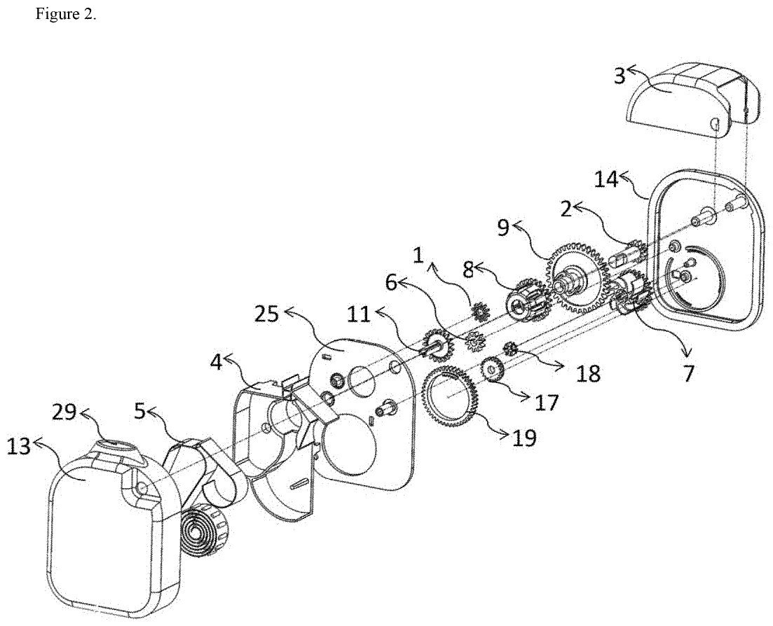

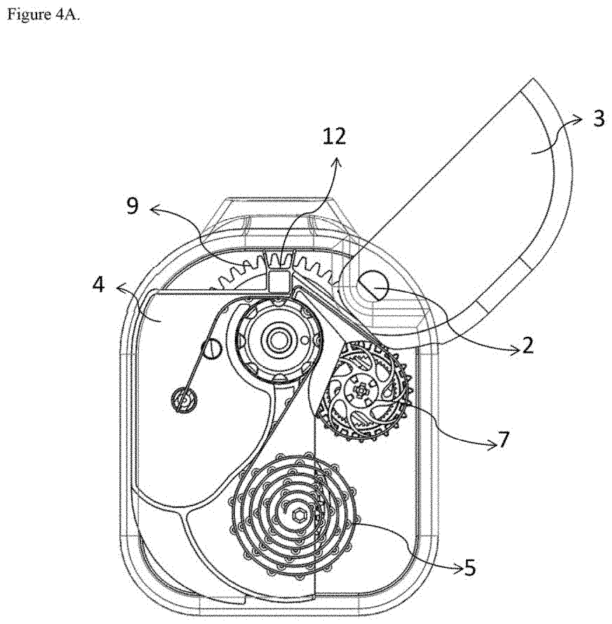

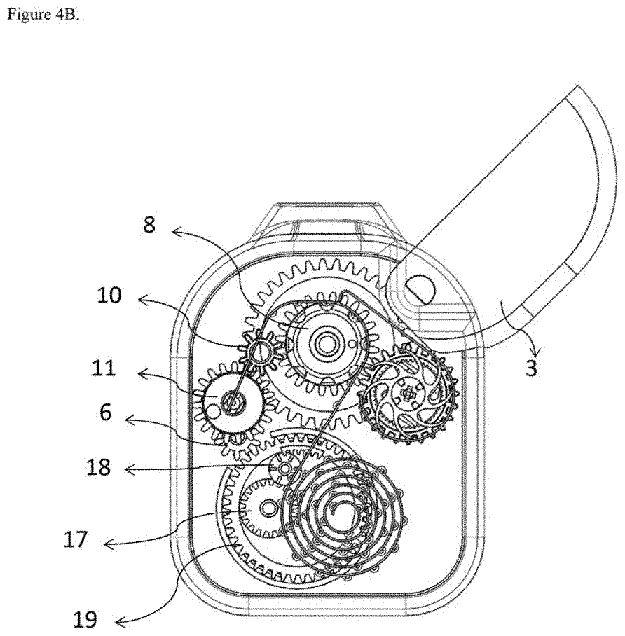

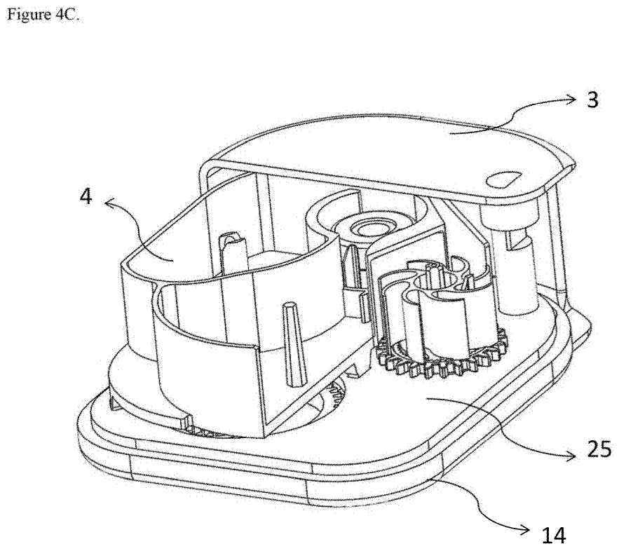

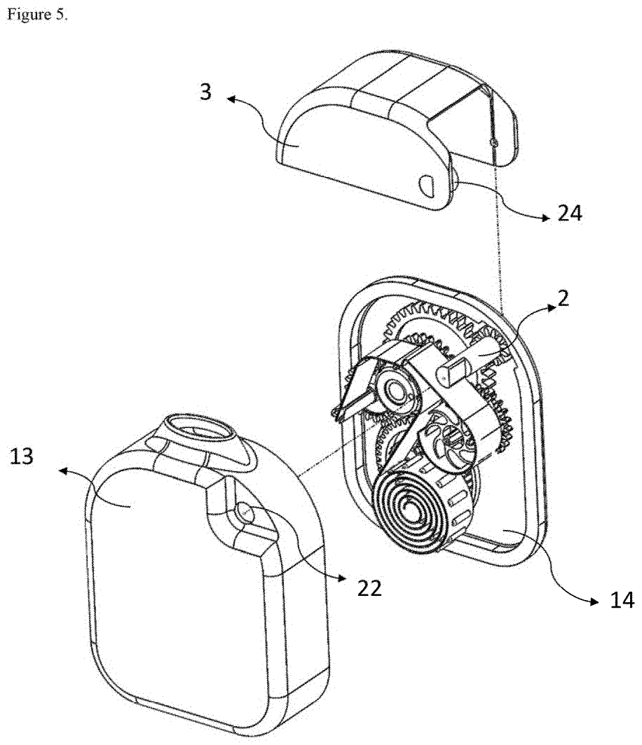

The top cover (13) (FIGS. 2, 5, 13-14) can locate the driver gear (2) via the top cover mounting hole (22) and can be permanently attached to the base cover (14). The top cover mounting hole (22) can allow the shaft (50) of the driver gear (2) to pass through (see FIGS. 4C and 13-15). The top cover (13) can include the mouthpiece (29).

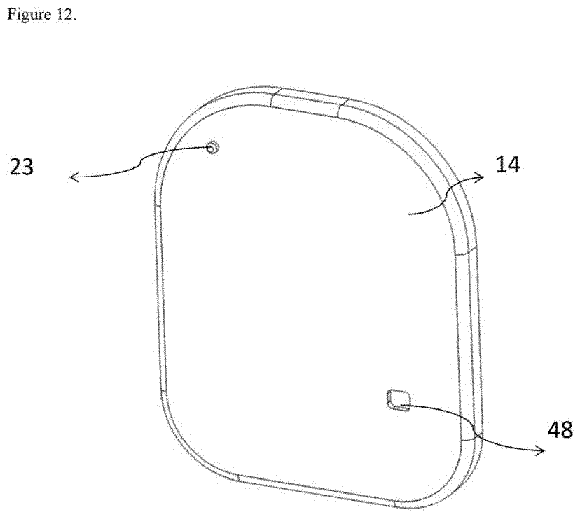

The base cover (14) (FIGS. 2, 11, 12 and 23) can house and locate all the components of the blister advancing mechanism and the dose indicator assembly. End boss (40) can locate the driver gear (2), and the central boss (42) can locate the cuvette ratchet (9). Studs (43), (44) and (45) can locate the components of the dose indicator assembly. Stud (43) can locate the dose indicator driving gear (6). Stud (44) can locate the dose indicator transfer wheel (18). Stud (45) can locate the dose indicator tens wheel (17). Locating Ring (46) can position the dose indicator units wheel (19) as it can ride on the bearing surface (47). The dose indicator stopper stud (106) can come in contact with the dose indicator stopper (105) of the dose indicator tens wheel (17) when all dose from the medicament dispenser are exhausted. The window (48) (see FIG. 12) shows both the dose indicator units wheel (19) and dose indicator tens wheel (17) to display the exact number of doses remaining inside the medicament dispenser.

The hinged cover (3) (FIGS. 2, 7A and 7B) can rotate about the axis passing through pivot hole (16) and boss (24). The axial movement of the hinged cover (3) can result in operation (actuation) of the medicament dispenser. The pivot hole (16) can rotate about the end pin (23) (see FIG. 12) on the base cover (14). The mounting hole (15) can engage with the flange (49) on the driver gear (2) and rotates it in the same direction as the direction of rotation of the hinged cover (3). The boss (24) can protect and cover the driver gear (2). The edge (30) (See FIG. 7B) on the hinged cover (3) can act as a stopper to prevent further rotation of the hinged cover (3) once it is completely operated (actuated). The hinged cover (3) can also include the compartment (80) (See FIG. 8) to accommodate a power source and/or electronic instruments.

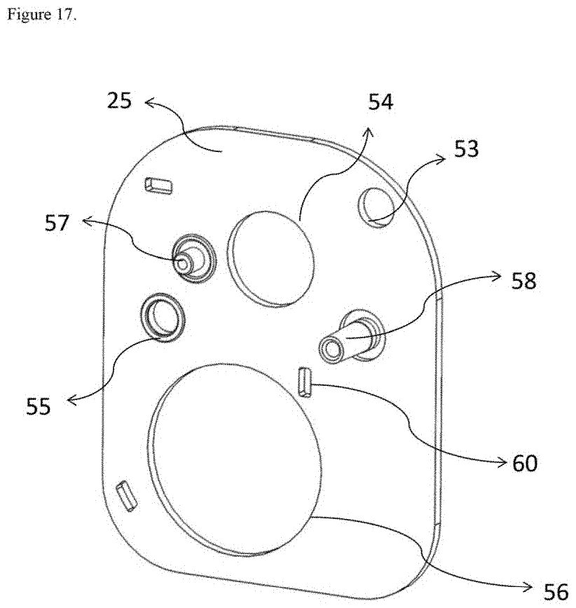

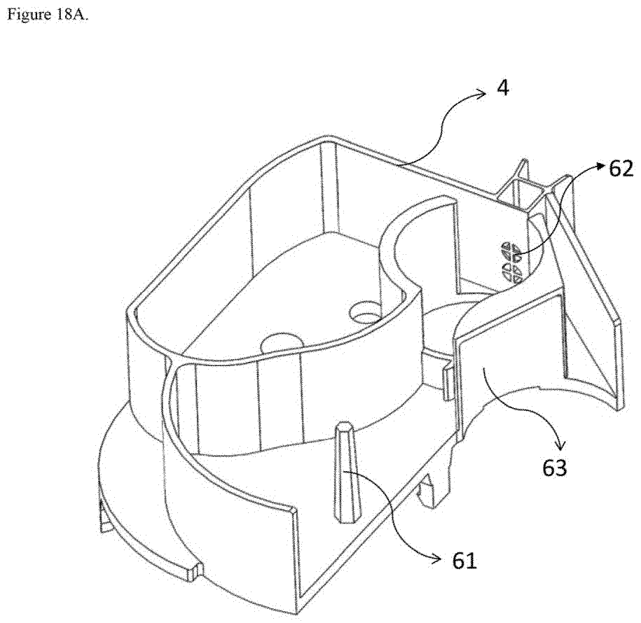

The inner housing support (25) (see FIG. 17) can be mounted and permanently affixed to the base cover (14). The inner housing support (25) has grooves (60) that locate and fix the inner housing assembly (4).

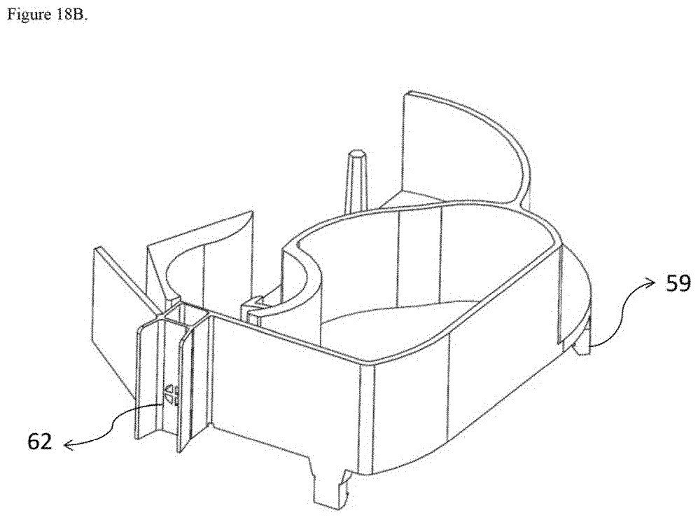

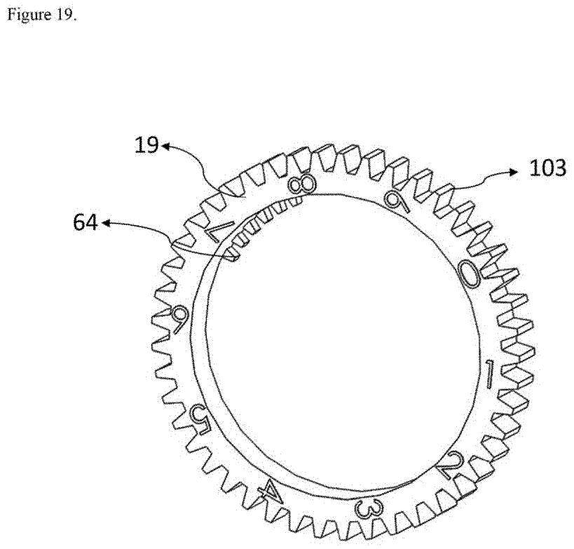

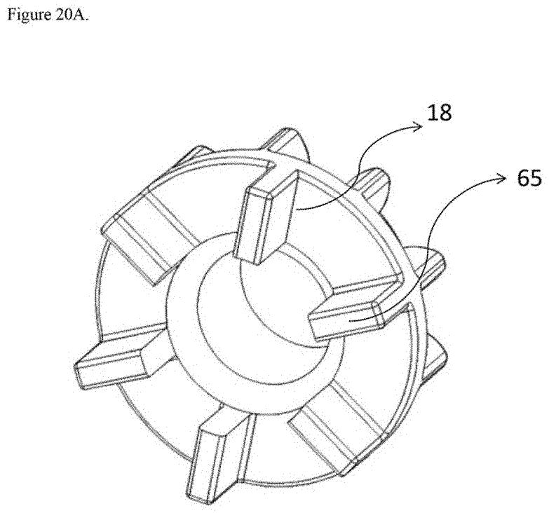

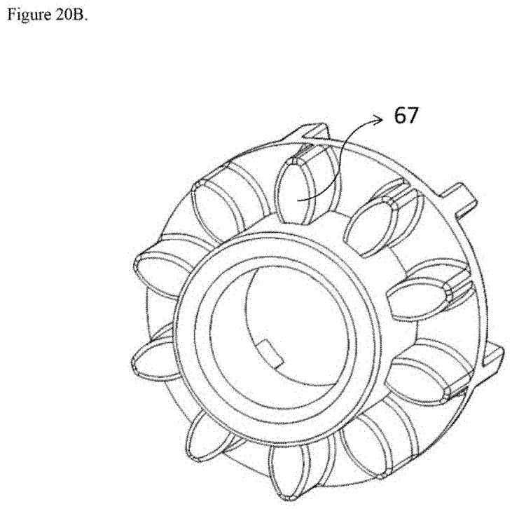

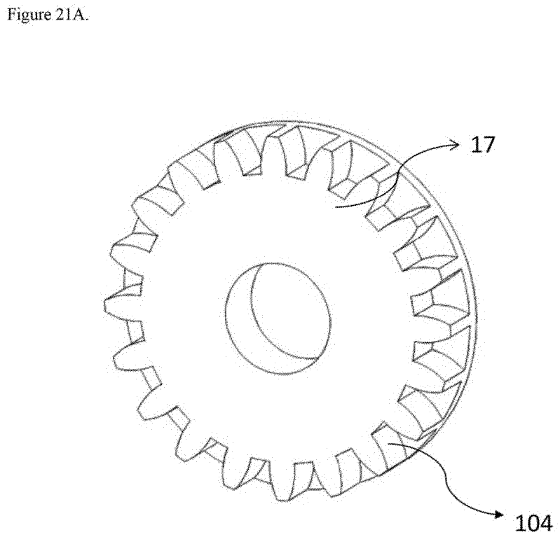

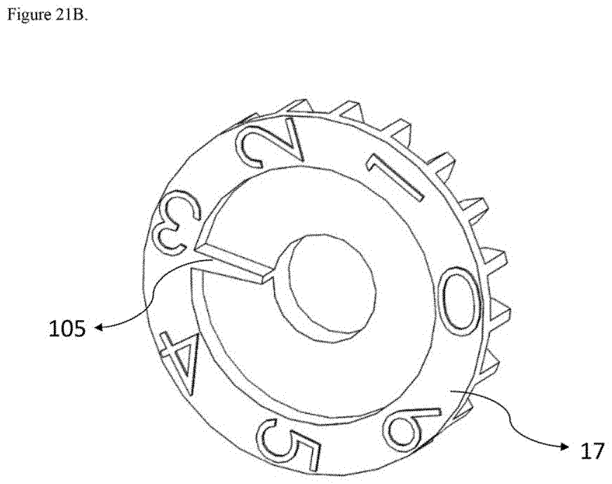

The inner housing assembly (4) (FIGS. 18A-18B) can be locked and placed on the inner housing support (25). The locking features are the snapping hooks (59) that lock in the groove (60) of the inner housing support. Openings (53), (54), (55) and (56) allow and provide bearing to the driver gear (2), blister cuvette (8), wrapping wheel (11) and the components of dose indicator assembly (17), (18) and (19) to pass through respectively. Stud (57) can locate the interim gear (10), and stud (58) can locate the winder assembly. The inner housing assembly (4) can hold the reel of blister (5) around the post (61). The inner housing assembly (4) provides the channels to guide the blister over the blister cuvette (8) as the reel of blister moves and peels during the process of operation/actuation of the medicament dispenser. The inner housing assembly (4) guides the two halves of the blister foil to wrap around the winder assembly (7) and the wrapping wheel (11). The inner housing assembly (4) can separate the rolled reel of blister (5) from the moving parts of the actuation mechanism. The inner housing assembly (4) also contains the mesh (62) to de-agglomerate the medicament when it leaves the blister during operation of the medicament dispenser. Guiding surface (63) can provide a frictionless surface for the reel of blister as it rolls on the winder assembly (7) during use.

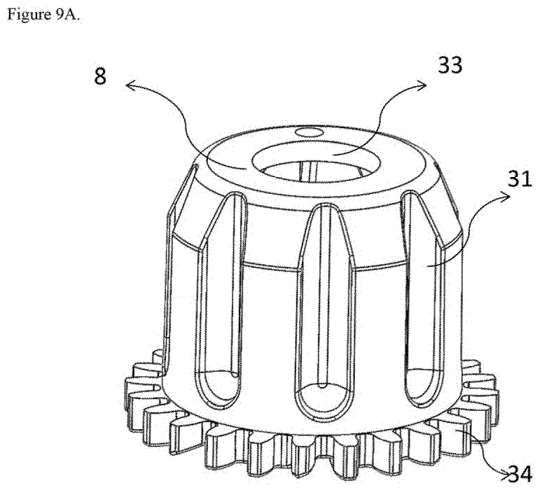

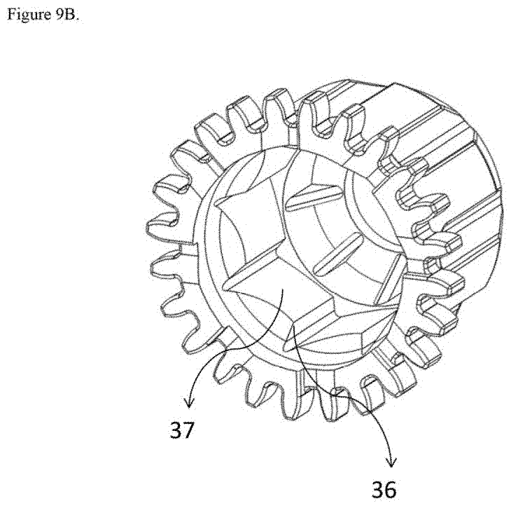

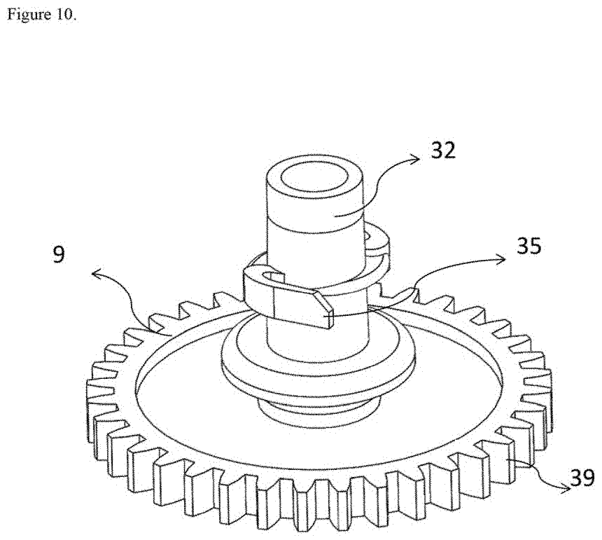

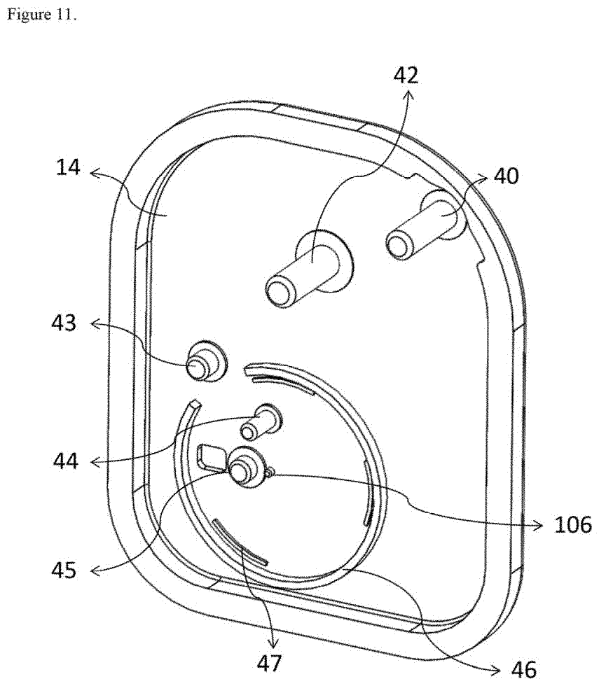

The blister cuvette (8) (FIGS. 9A-9B) can locate each blister cavity (41) during the operation of the medicament dispenser. The blister cuvette (8) helps to locate and align the blister cavity along the flow channel/opening (12) when the mechanism is operated (actuated). The blister cuvette (8) can include eight equally spaced individual cuvettes (31) around the circumference. The cuvette ratchet (9) is located within the blister cuvette (8) and is responsible for discrete angular movement of the blister cuvette (8). The blister cuvette (8) rotates about the central boss (32) of the cuvette ratchet (9). The central boss (32) passes through and locates the central hole (33) on the blister cuvette (8). The spur gears (34) on the blister cuvette (8) can drive the winder assembly (7) and the wrapping wheel (11). The ratchets (35) rest on the contacting surface (36) on the blister cuvette (8). The ratchets (35) can drive the blister cuvette (8) in one direction only through the contacting surface (36). When the direction of rotation of the cuvette ratchet (9) reverses while closing the medicament dispenser, it rides on the non-contacting surfaces (37). The spur gears (39) on the cuvette ratchet (9) engage with the driver gear teeth (21).

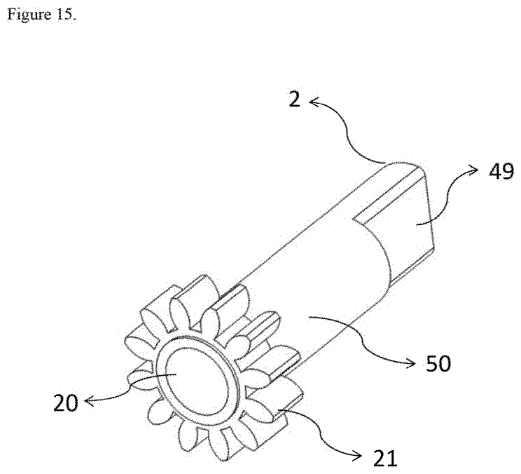

The driver gear (2) (see FIG. 15) can be driven by the hinged cover (3) via the flange (49). The shaft (50) passes through and is guided by end boss (40) on the base cover (14) that passes through bearing (20) on the driver gear (2).

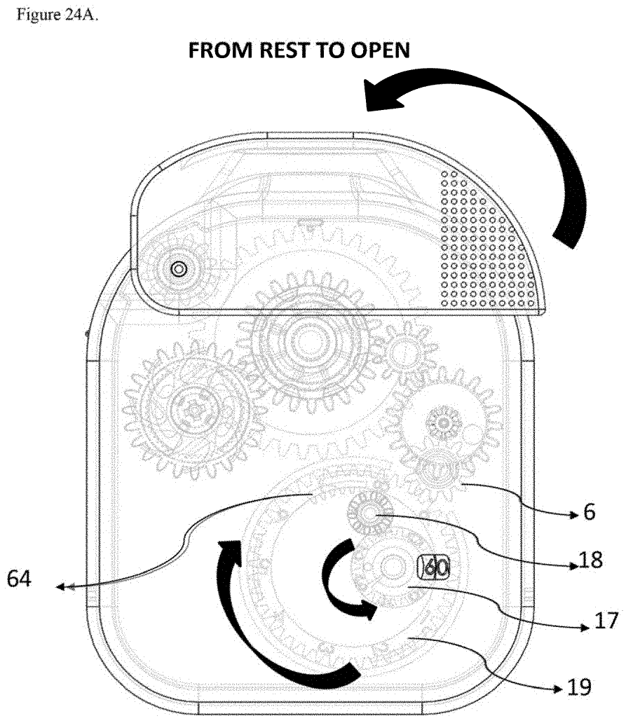

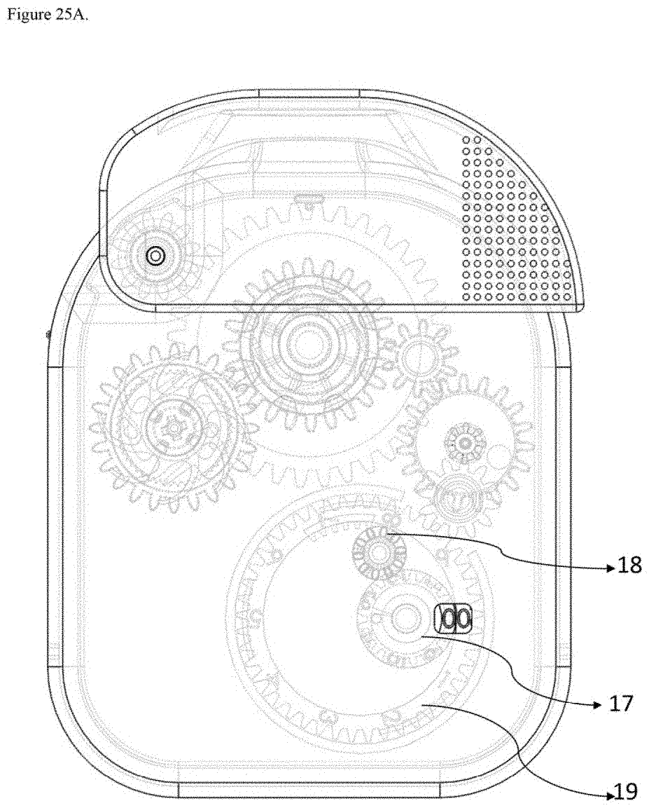

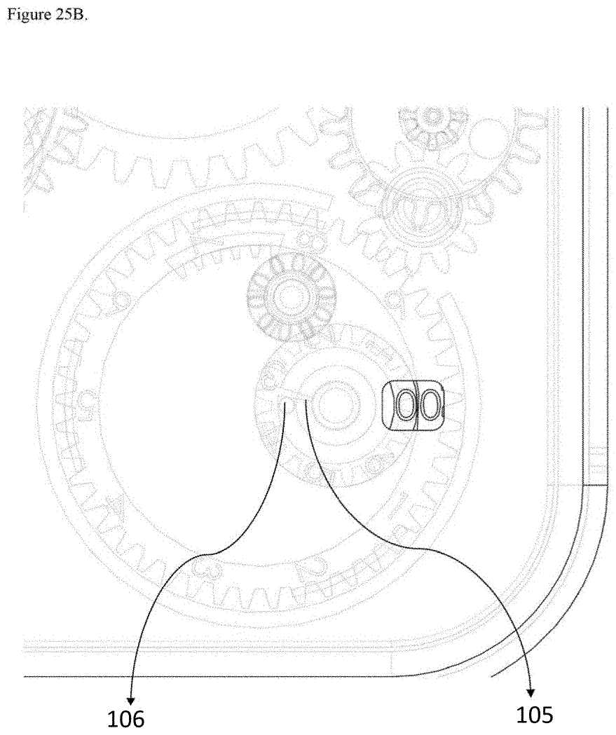

The dose indicator assembly (FIGS. 23, 24A-24C, 25A-25B) can include the dose indicator driving gear (6), the dose indicator units wheel (19), dose indicator transfer wheel (18) and the dose indicator tens wheel (17). The dose indicator units wheel (19) can be driven by the dose indicator driving gear (6) via the dose indicator units wheel gear teeth (103). The driving gear (6) in turn can be rotated by the gear (66) of the wrapping wheel (11). The dose indicator units wheel (19) can drive the dose indicator tens wheel (17) through the dose indicator transfer wheel (18). The inner gears (64) on the dose indicator units wheel (19) can rotate the teeth (65) on the dose indicator transfer wheel (18) when it completes the rotation of ten doses. The spur gears (67) on dose indicator transfer wheel (18) increment the dose indicator tens wheel (17) via the dose indicator tens wheel gear teeth (104) to display the next lower tenth dose. The inner gears (64) on the dose indicator units wheel (19) do not come in contact with the dose indicator transfer wheel (18) until ten doses are completed. The dose indicator stopper (105) on the dose indicator tens wheel (17) comes in contact with the dose indicator stopper stud (106) on the base cover (14) when all dose from the medicament dispenser are exhausted and the dose indicator reads ‘00’.

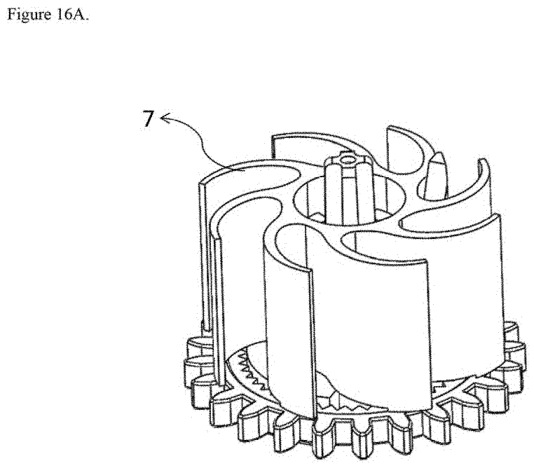

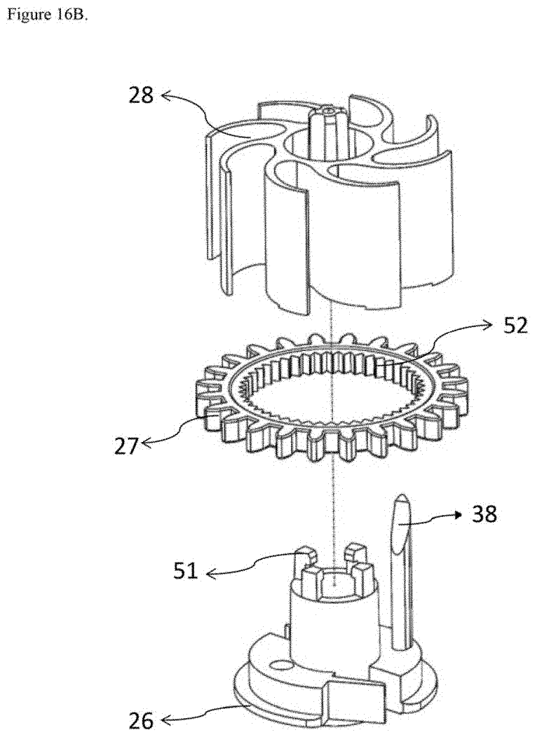

The winder assembly (7) (FIGS. 16A-16B) can include the winder wheel (28), the winder gear (27) and the ratchet (26). The winder assembly (7) can be driven by the blister cuvette (8). Locking pins (51) can lock with the winder wheel (28). The reel of blister (5) is rolled on the winder wheel (28) during assembly and a pre-tension is provided by the rotation of the winder wheel (28). The assembly of the winder wheel (28) and the ratchet (26) ratchet on the inner gear (52) of the winder gear (27). Once the winder assembly (7) is locked in placed, the winder wheel (28), winder gear (27) and the ratchet (26) rotate together.

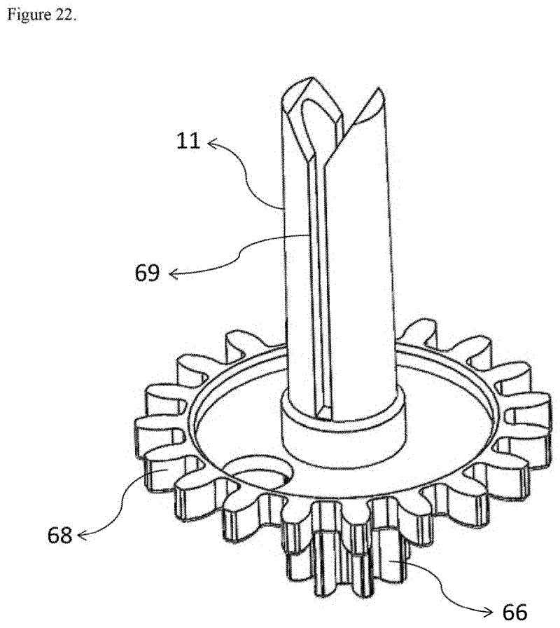

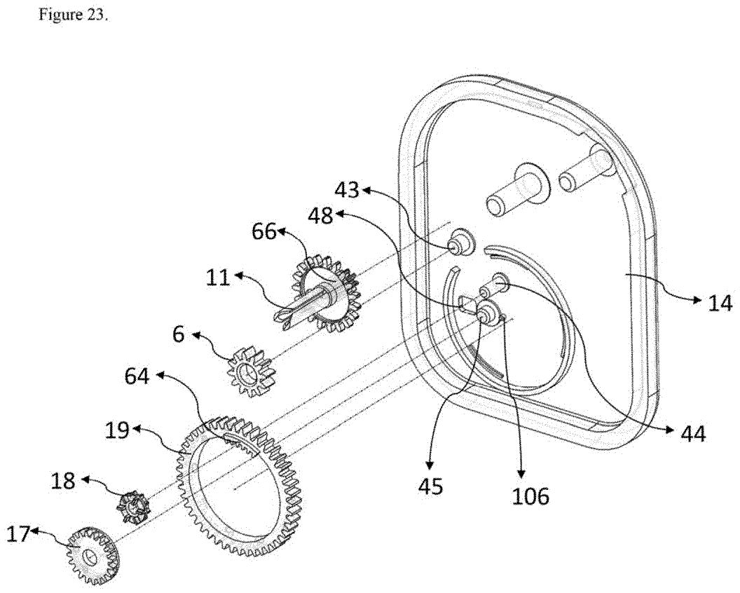

The wrapping wheel (11) (FIG. 22) can be driven by the blister cuvette (8) via the spur gears (68). The open end (71) can be located with the slit (69) and wraps around the wheel as it rotates during actuation. Gear (66) drives the dose indicator driving gear (6) during actuation. The wrapping wheel (11) rotates in the opposite direction of the blister cuvette (8) via the interim gear (10).

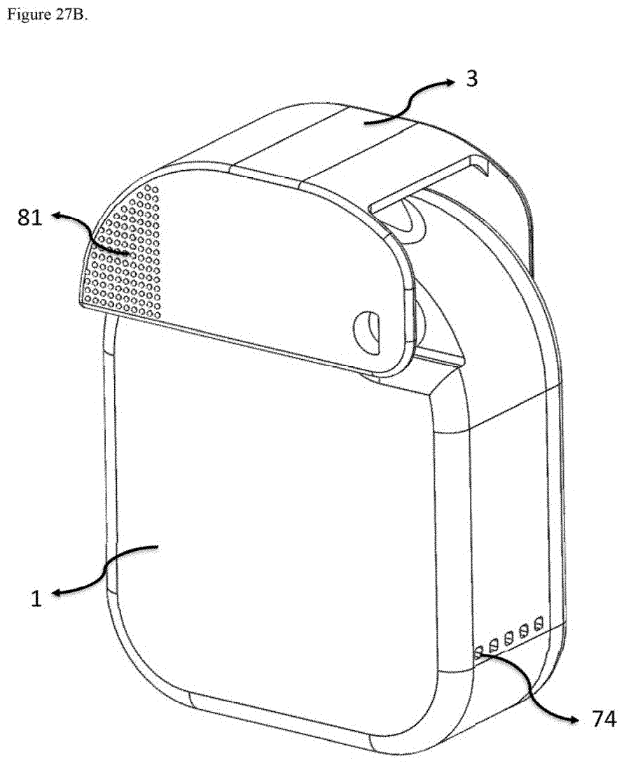

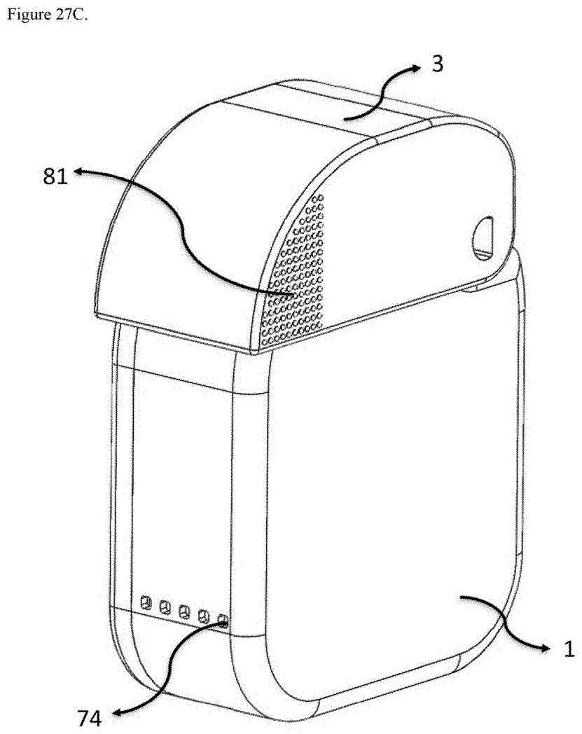

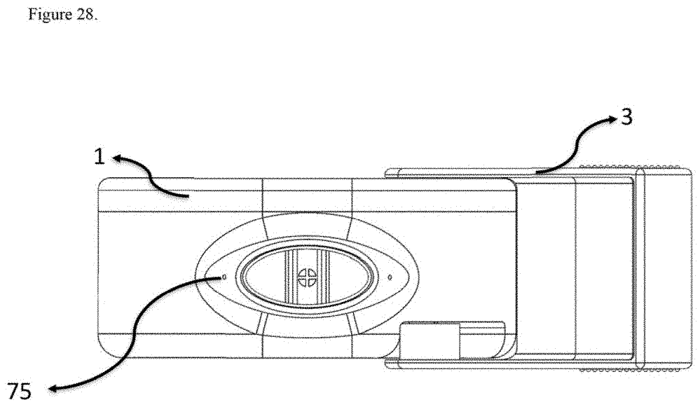

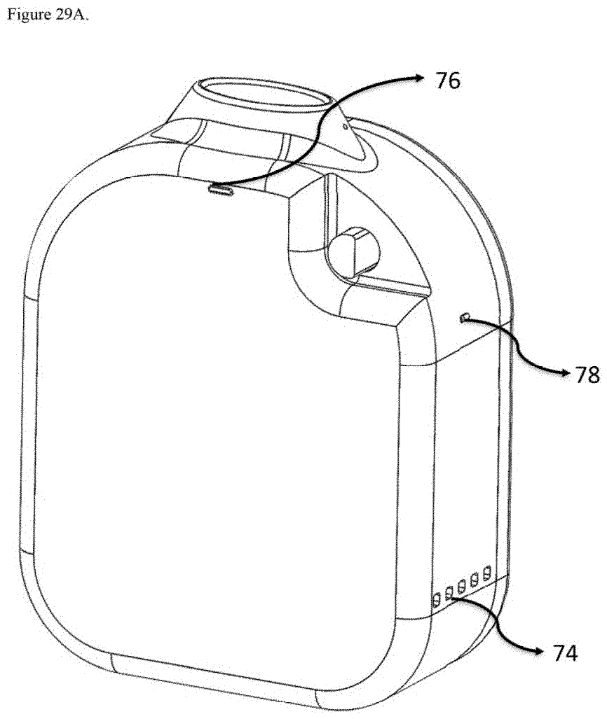

The air inlet vent holes (74) can be located discretely on the top cover to provide for an air inlet through the medicament dispenser. The mouthpiece vent holes (75) provide for by-pass air outlet (FIGS. 27A-27C and 28).

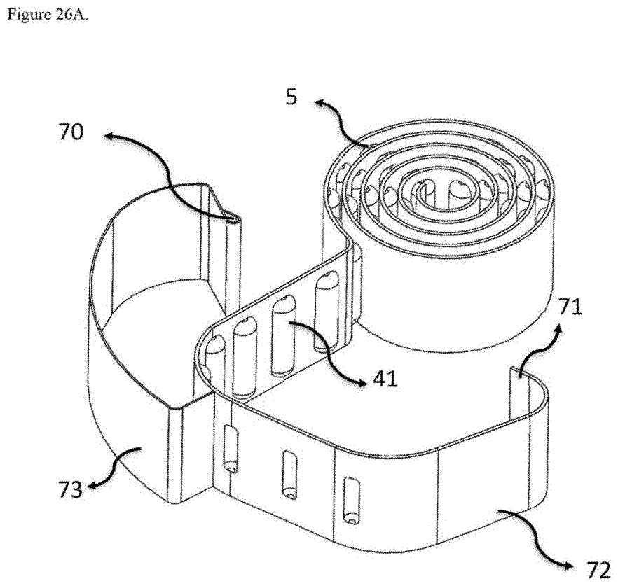

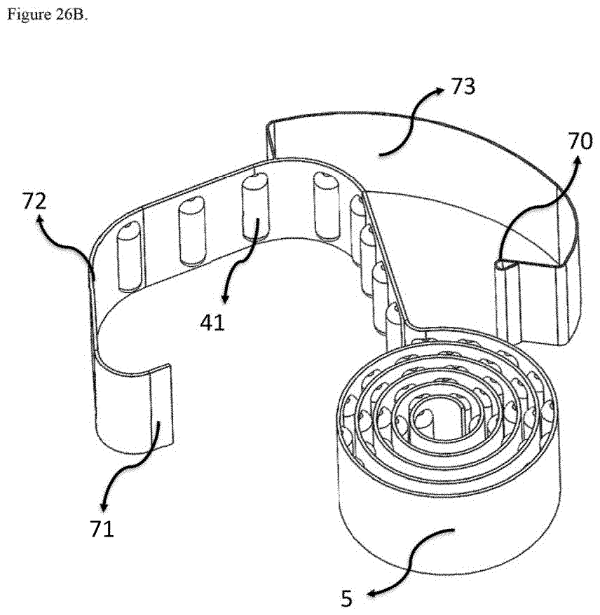

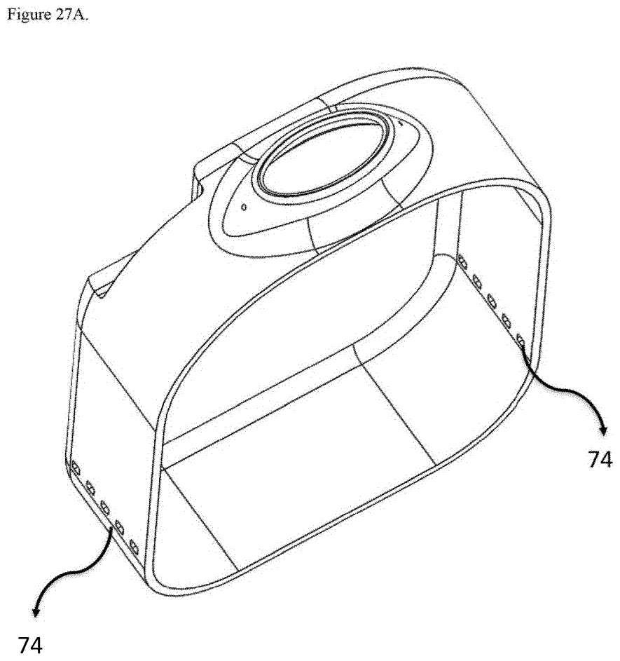

The reel of blisters (5) can incorporate a lid sheet (73), and a base sheet (72) which contains individual blister cavities (41) (FIGS. 26A-26B). The reel of blisters (5) can be wound around the post (61). One end of the reel of blister has a sealed loop (70) that can be placed in the blister post (38). The open end (71) of the reel of blister (5) can be placed in the slit (69) of the wrapping wheel (11).

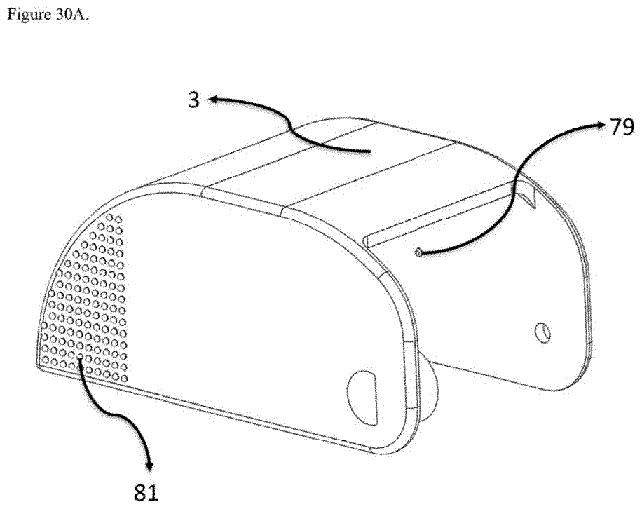

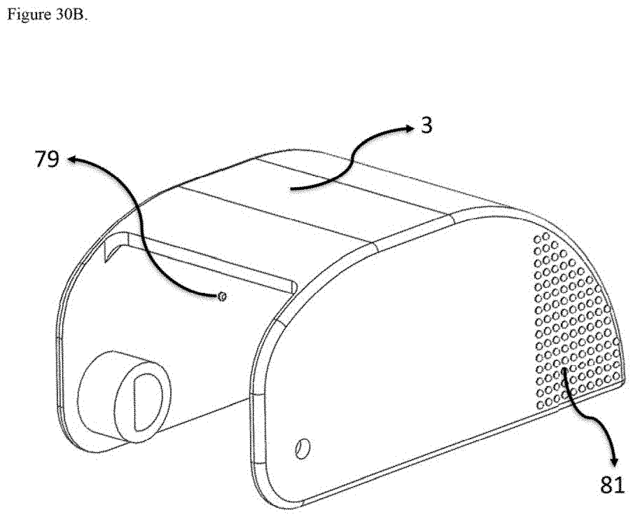

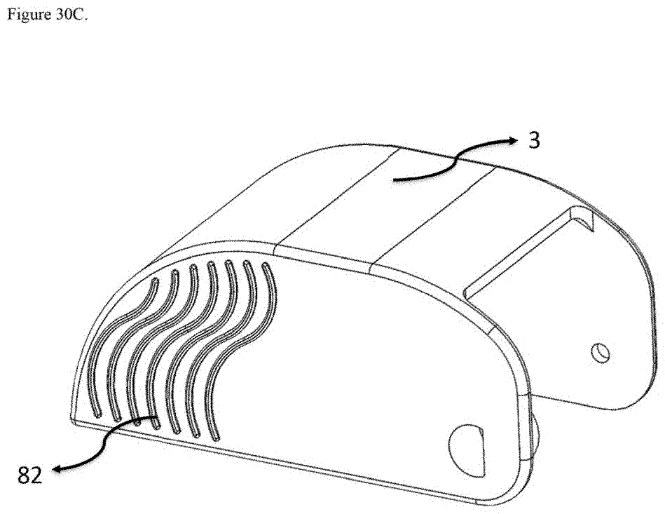

The dotted gripping feature (81) or the wave gripping feature (82) (FIGS. 30A-30C) can be provided for an efficient gripping mechanism to operate the hinged cover (3).

The Window (48) provides a unified view of the dose indicator tens wheel (17) and the dose indicator units wheel (19) for user feedback on number of remaining doses.

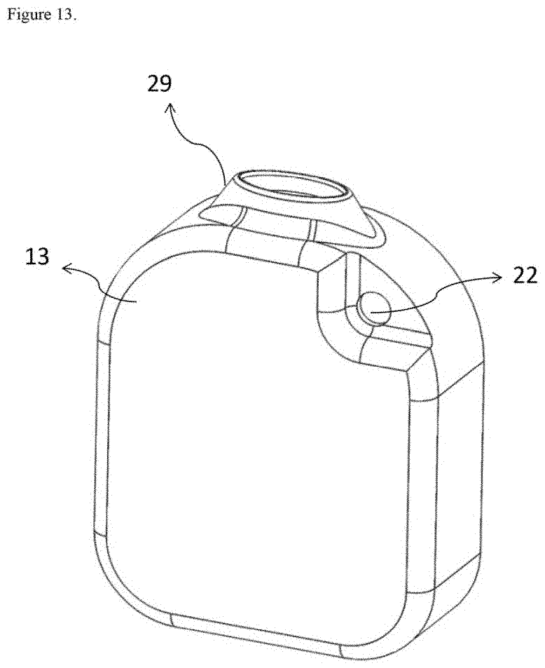

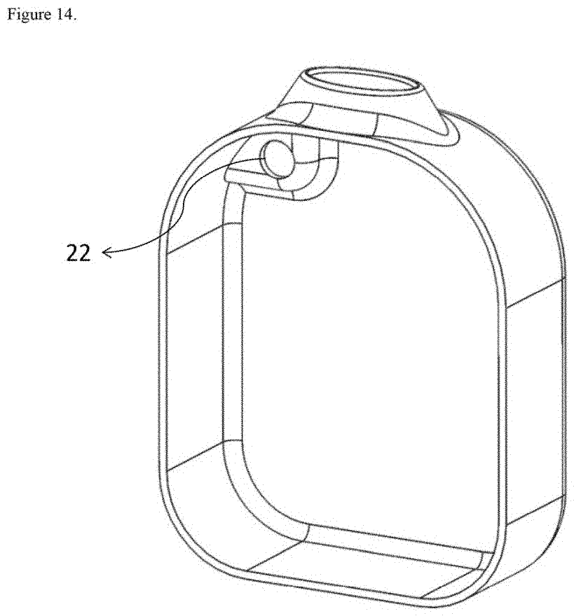

The mouthpiece (29) (FIGS. 2 and 13) allows the user to receive the contents of the reel of blisters (5) by placing the mouthpiece (29) inside the oral cavity. The mouthpiece (29) allows the contents of the reel of blisters (5) to pass through the flow channel (12). The mouthpiece (29) can be covered by the hinged cover (3) when not in use.

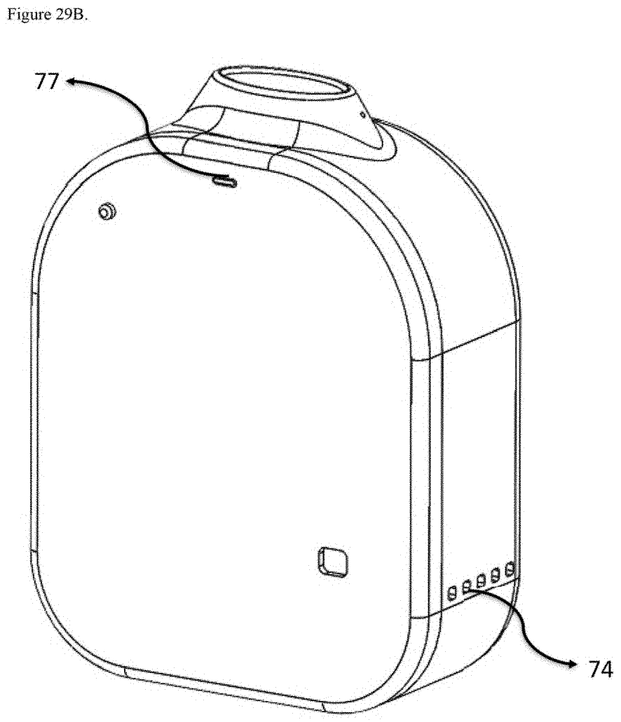

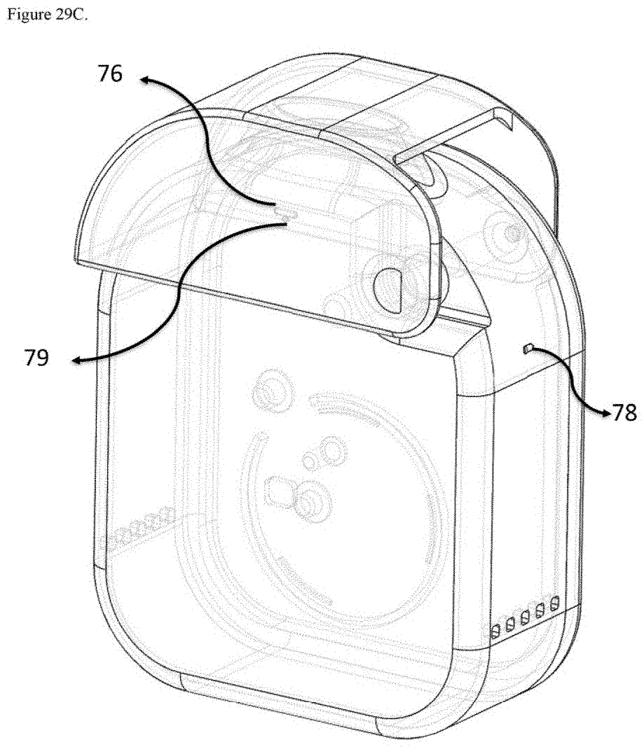

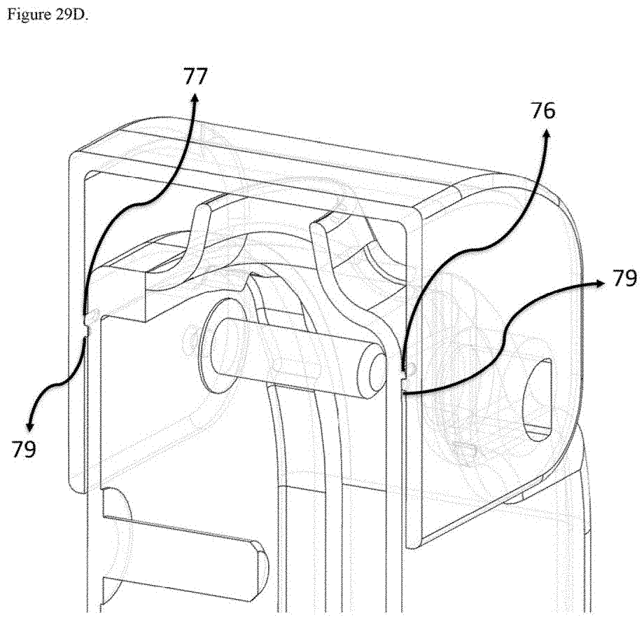

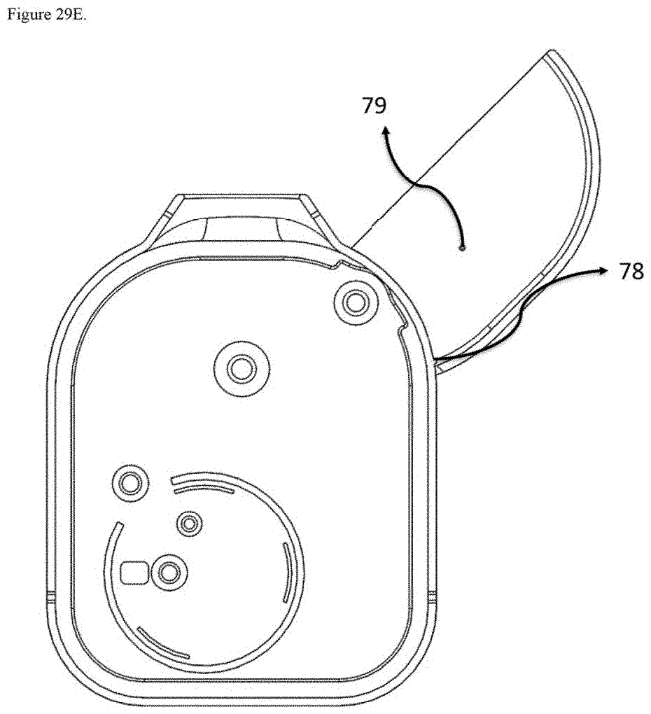

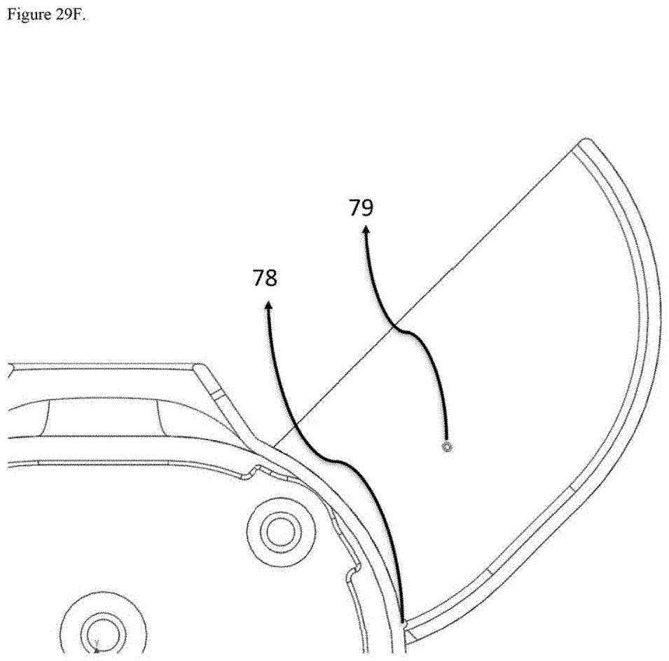

A locking/snapping mechanism for locking/snapping the hinged cover on the assembly can be provided (FIGS. 29A-29C and 29D-29F). The ridge (76) on the top cover (13) and the ridge (77) on the base cover (14) provide for a locking mechanism for the hinged cover (3) on the assembly via corresponding centrally located ridges (79) on the hinged cover (3). The ridges (79) can be located symmetrically on both the surfaces of the hinged cover (3). The ridge (79) snaps over corresponding ridges (76) and (77) on both the top cover (13) and base cover (14), respectively. This mechanism can prevent unintended and accidental operation/opening of the hinged cover.

From a user's perspective, the operation/opening of the hinged cover is a continuous movement without any intermediate position until the hinged cover (3) cannot be rotated any further. Once the hinged cover is completely operated/opened, the hinged cover (3) locks in place with the aid of the ridge (78) on the top cover (13). This mechanism can provide instructions and a feedback to the user to operate/open the hinged cover completely and prevents any unintended and accidental movement of the hinged cover during the dosing operation of the medicament dispenser. This mechanism can also provide a haptic (i.e. snap) and an auditory feedback to the user once the hinged cover is completely operated/opened, indicating that the medicament dispenser is ready for dispensing the dose.

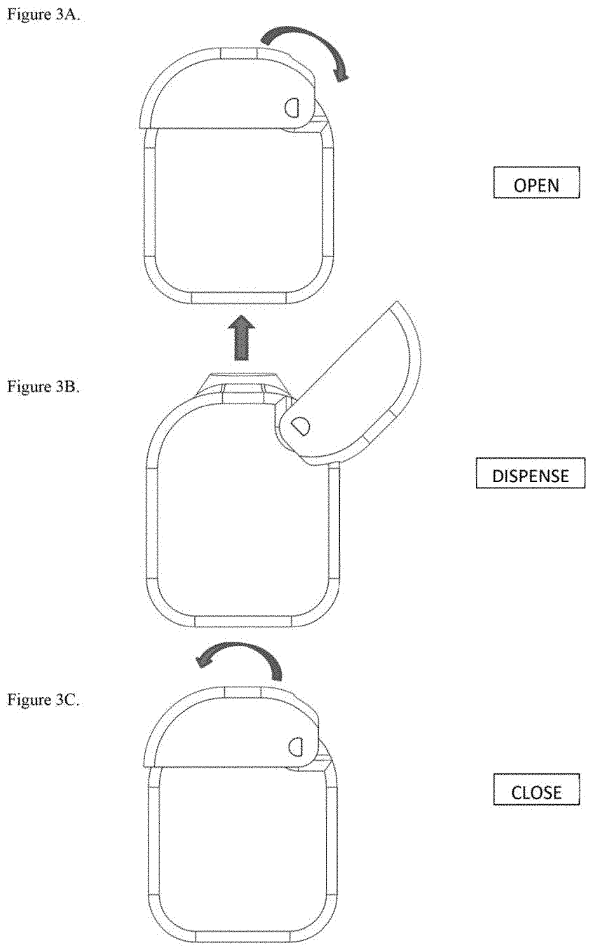

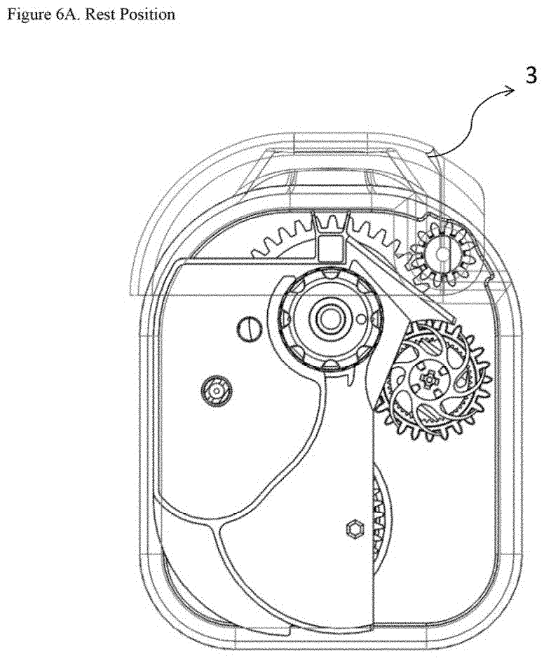

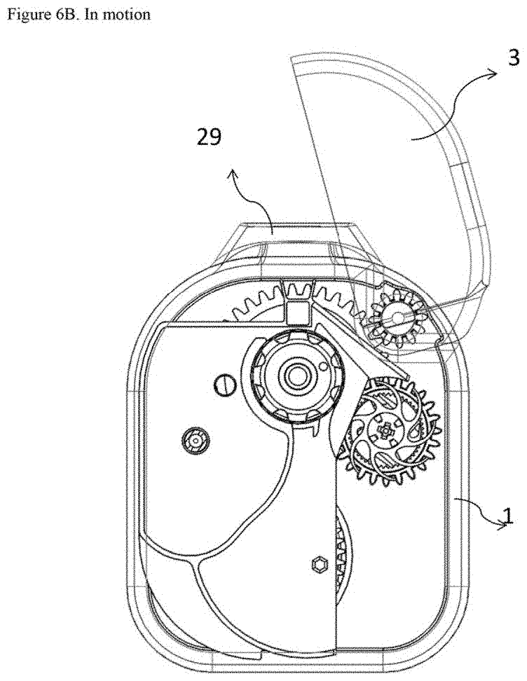

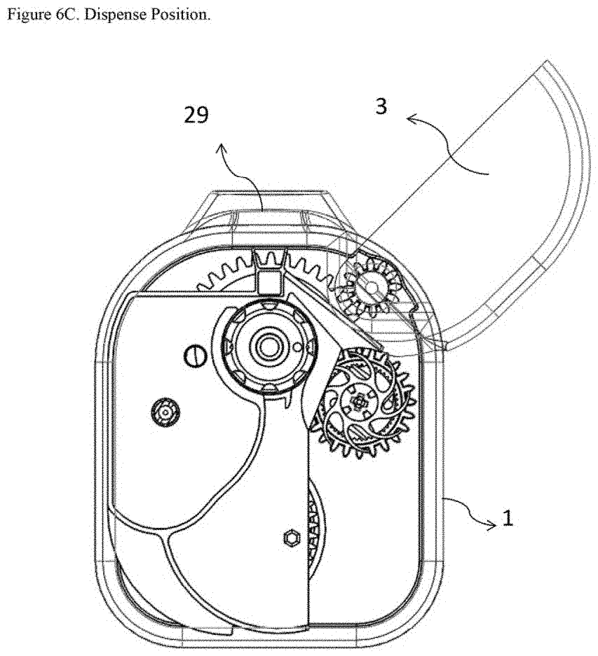

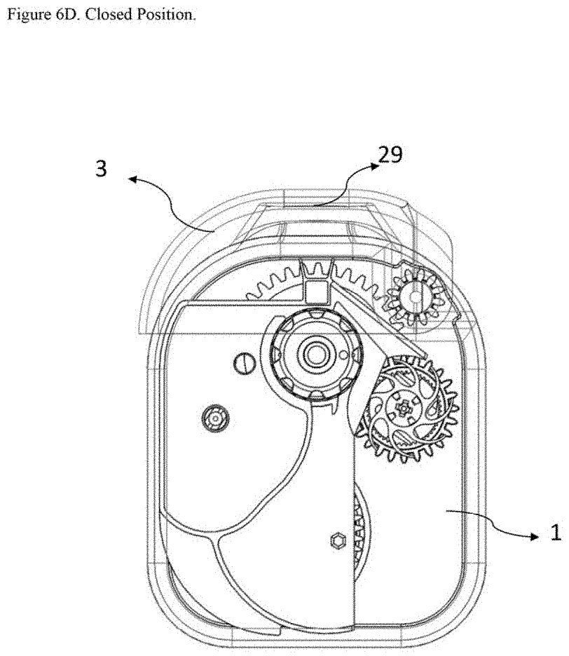

The mechanism of operation of the medicament dispenser is presented in FIGS. 3A-3C and 6A-6D. In the initial (rest) state the hinged cover (3) covers the flow channel (12) and mouthpiece (29) of the assembly (1). When the user opens (rotates) the hinged cover (3), the hinged cover (3) rotates the cuvette ratchet (9) via a driver gear (2). The blister cuvette (8) rotates synchronously and uni-directionally with the cuvette ratchet (9), thereby advancing the blister cavities (41) from the reel of blisters (5). Advancement of reel of blisters (5) causes the leading blister cavity to be peeled open and brought into communication with the flow channel (12), which itself communicates with mouthpiece (29), and the medicament of the blister cavity are available for dispensing. To access the medicament of the opened cavities of reel of blisters the user places the mouth on the mouthpiece (29) and orally inhales. This results in air enters the device through the air inlet vent holes (74) and exits the device through the mouthpiece and through the mouthpiece vent holes (75). In turn, this results in the medicament contained within the opened cavities being aerosolized and guided through the flow channel and hence to the user as an inhaled medicament dose.

When the user closes (rotates) the hinged cover in the direction opposite to that of the first step it covers the mouthpiece (29) and the flow channel (12). At this stage the motion of the hinged cover (3) rotates the cuvette ratchet (9) without advancing the reel of blisters. The ratcheting mechanism prevents the rotation of the blister cuvette (8) in the opposite direction.

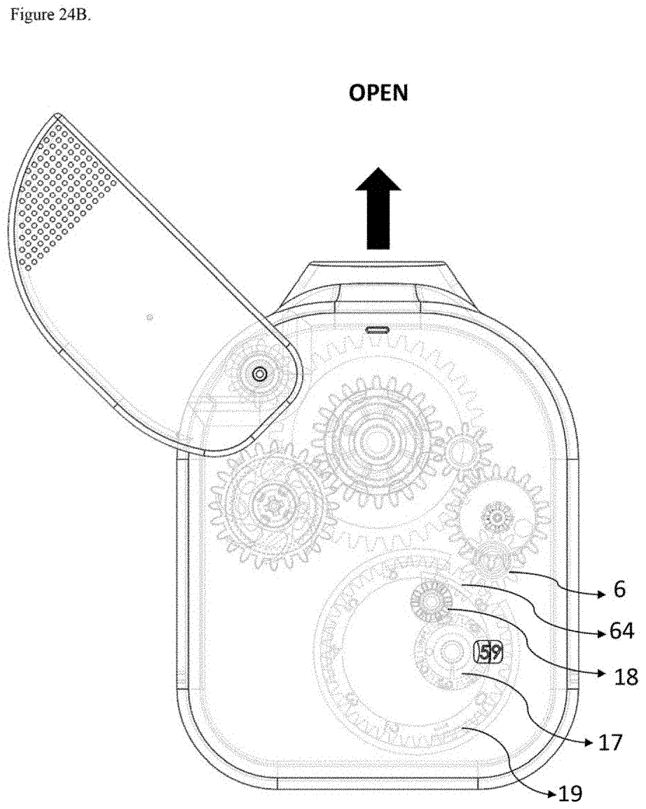

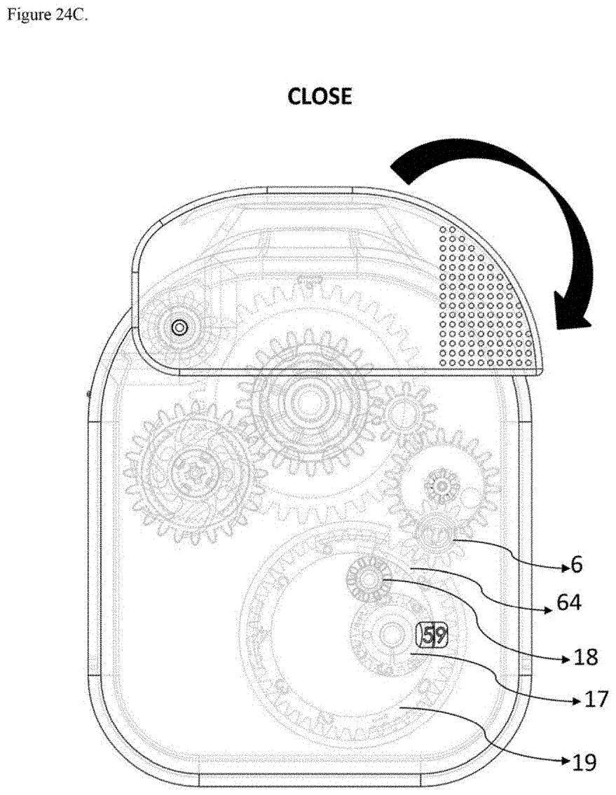

The dose indicator assembly can be driven by the wrapping wheel (11). The hinged cover (3) is opened (rotated) in response to the user action causing the dose indicator driving gear (6) to rotate. The gear (66) of the wrapping wheel (11) rotates the dose indicator driving gear (6) which in turn rotation of the dose indicator units wheel (19) in the clockwise direction. The gearing of the wrapping wheel (11), the dose indicator driving gear (6) and the dose indicator units wheel (19) is arranged such that when the user opens (rotates) the hinged cover (3), the dose indicator units wheel (19) rotates and a single indicium thereon is advanced (i.e. the ‘units’ count moves on one unit).

Where the previous visible count was x0 (e.g. 60, 50, 40 or 30 or 20), the counting action resulting from the use operation is subtly different. Once again, the hinged cover (3) is rotated in response to user action causing the dose indicator driving gear (6) to rotate and in turn rotation of the dose indicator units wheel (19) and the ‘unit’ indicium moves on from ‘0’ to ‘9’ . This rotation of the dose indicator units wheel (19) however, also brings the inner gear (64) into meshed relationship with the teeth (65) of the dose indicator transfer wheel (18) such that the dose indicator transfer wheel (18) rotates in the clockwise direction and in turn, the dose indicator tens wheel (17) rotates in the counter clockwise direction. As shown in FIGS. 24A and 24B, the gearing of the relevant wheels 19, 18, 17 is arranged such that the dose indicator tens wheel (17) rotates and a single indicium thereon is advanced from ‘6’ to ‘5’ (i.e. the ‘tens’ count moves down exactly one unit).

It will be appreciated that the above usage of the counter has been described in terms of a dose indicator assembly arranged to count downwards, but that the indicator assembly may be straightforwardly modified to count upwards.

The dose indicator stopper (105) comes in contact with the dose indicator stopper stud (106) on the base cover (14) when the visible count reads ‘00’. At this time, no doses are left in the medicament dispenser. At this time, the dose indicator mechanism along with the blister advancing mechanism can be locked.

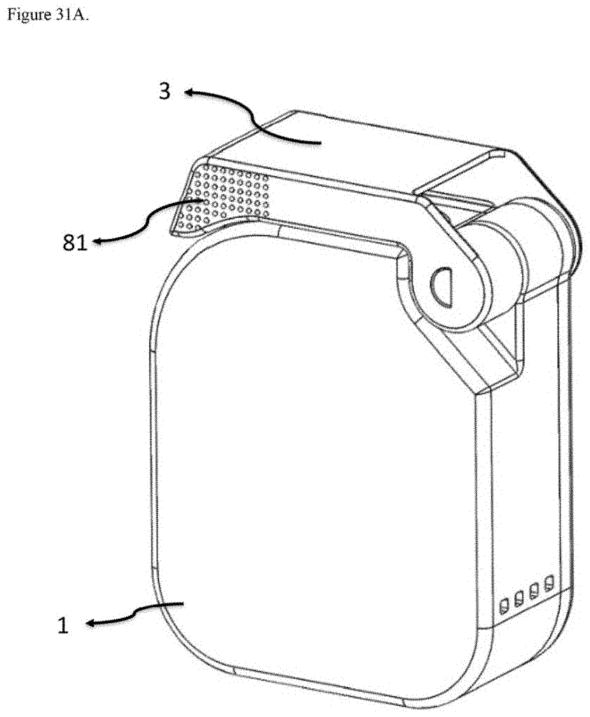

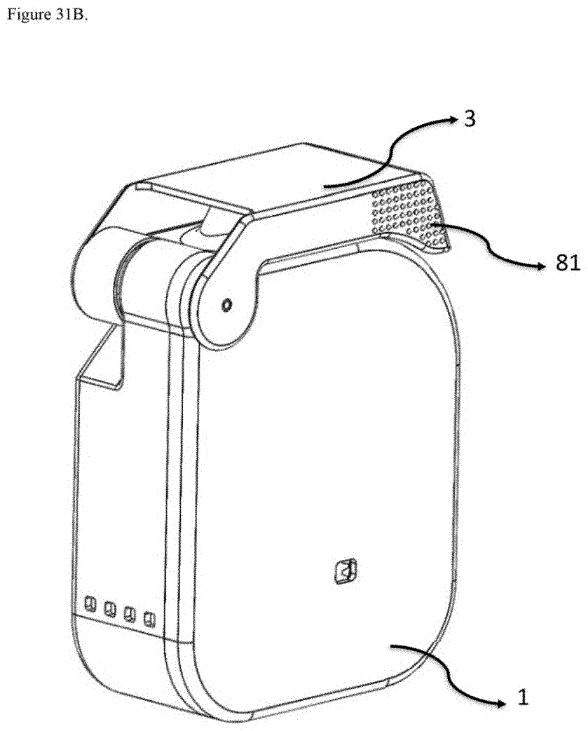

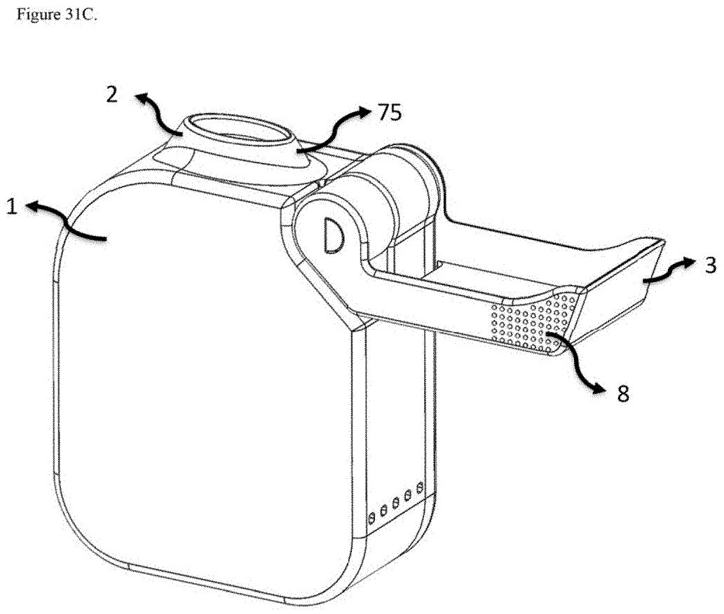

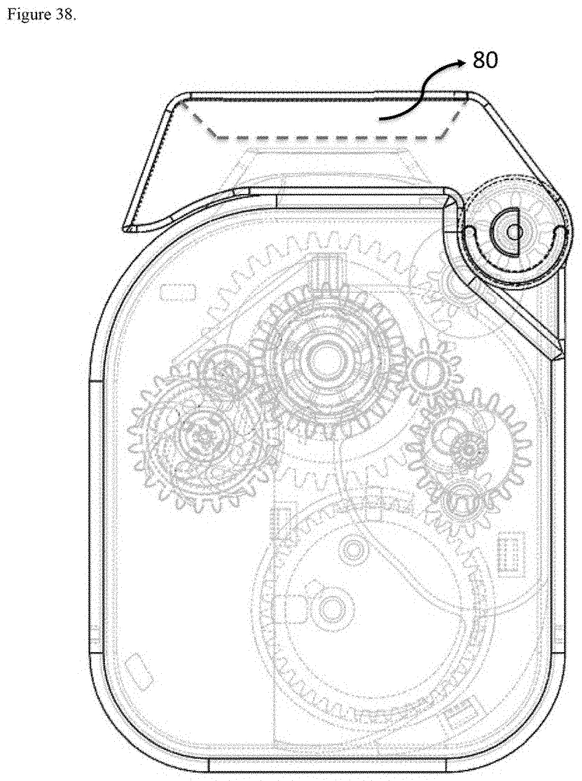

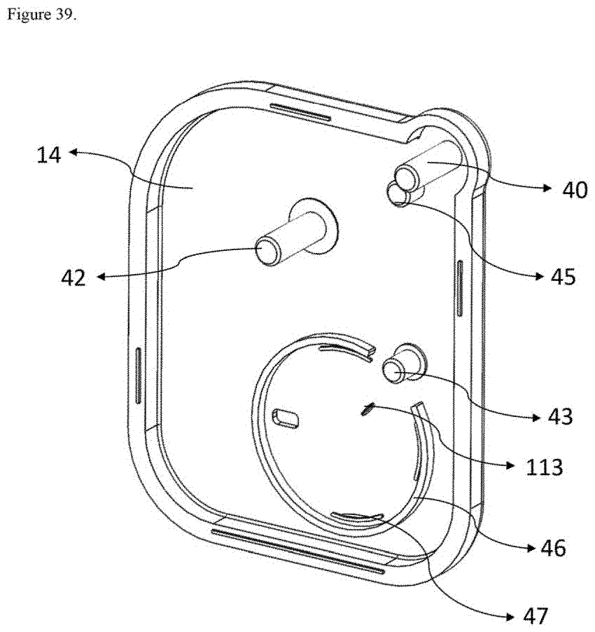

FIGS. 31A-31C illustrate different views of the medicament dispenser according to another aspect of the disclosure. The medicament dispenser can include an assembly (1) which houses a reel of blisters (5) and the blister advancing mechanism. The assembly (1) can include the base cover (14) and the top cover (13) encasing all the moving components within the assembly. The blister advancing mechanism can be operated (actuated) via the hinged cover (3). The hinged cover (3) can be mechanically mounted to the assembly (1). The hinged cover (3) (see FIGS. 37A and 37B) can include the boss (24) to prevent the hinged cover (3) from getting disassembled from the assembly during operation of the medicament dispenser.

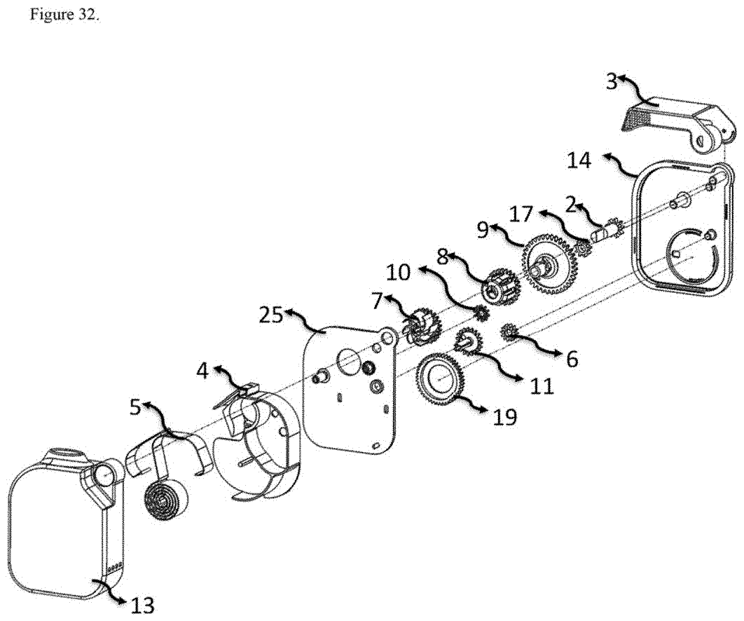

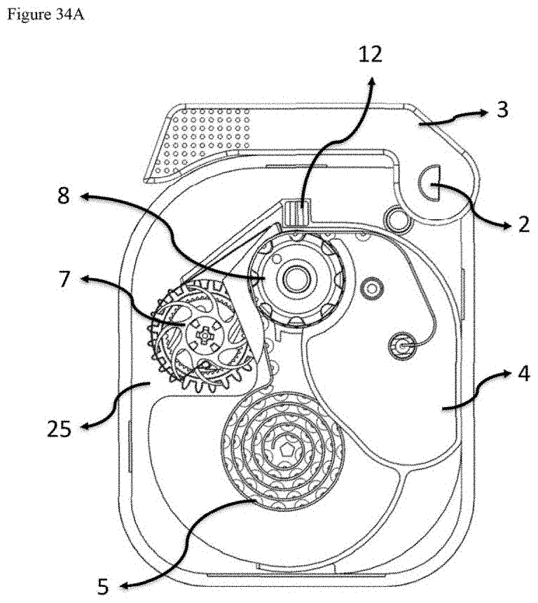

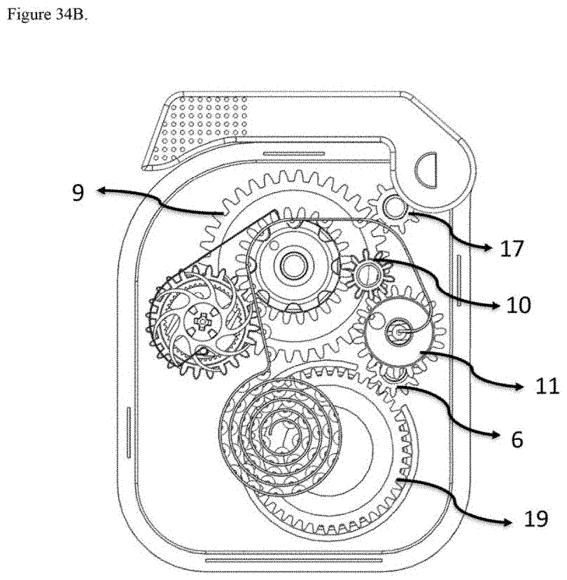

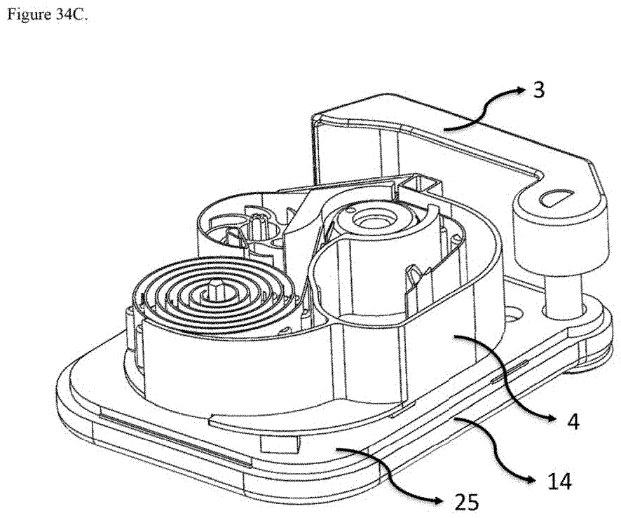

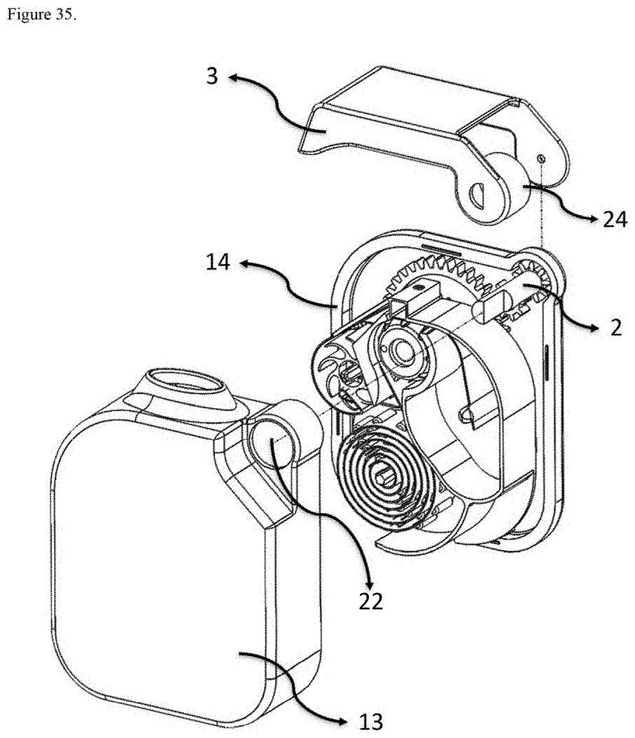

The top cover (13) (FIGS. 32, 35, 41 and 42) can locate the driver gear (2) via the top cover mounting hole (22) and can be permanently attached to the base cover (14). The top cover (13) can also include a mouthpiece (29) (FIGS. 31C, 32, 36B, 36C, 36D, 41, 40). The top cover mounting hole (22) can allow the shaft (50) of the driver gear (2) to pass through (see FIGS. 34C, 41-42).

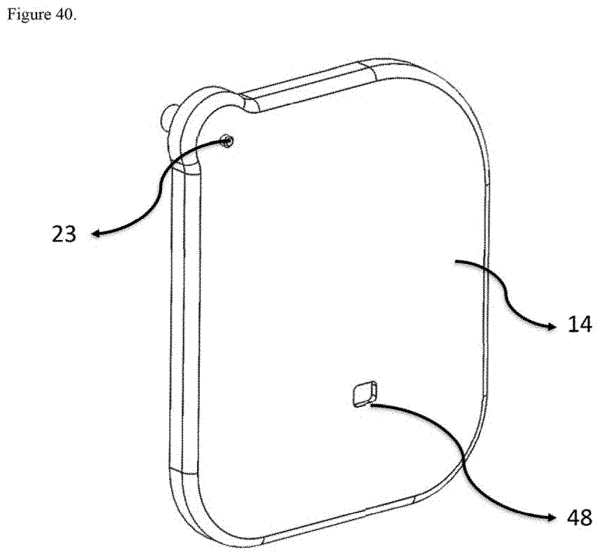

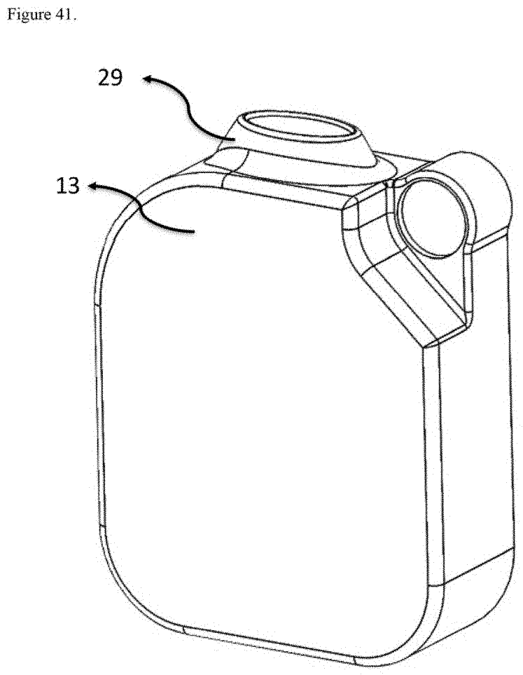

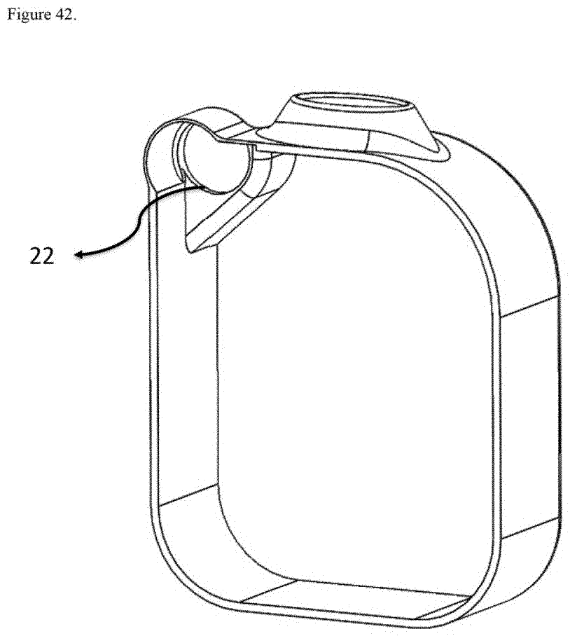

The base cover (14) (FIGS. 32, 35, 39 and 40) can house and locate all the components of the blister advancing mechanism and the dose indicator wheel (19). End boss (40) can locate the driver gear (2), and the stud (45) can locate the interim driver gear (17). The central boss (42) can locate the cuvette ratchet (9). Stud (43) can locate the dose indicator driving gear (6). Locating Ring (46) can position the dose indicator wheel (19) as it rides on the bearing surface (47). The pivot hole (16) on the hinged cover (3) can mate with the end pin (23). The base cover (14) can include a dose indicator stopper stud (113) locks the dose indicator wheel (19) at ‘0’ when all dose from the medicament dispenser are exhausted. The Window (48) provides a view of the dose indicator wheel (19) for user feedback on number of remaining doses (FIG. 40).

The hinged cover (3) (FIGS. 31A-31C, 32, 37A-37B) can rotate about the axis passing through pivot hole (16) and boss (24). The axial movement of the hinged cover (3) can result in operation (actuation) of the medicament dispenser. The pivot hole (16) can rotate about the end pin (23) on the base cover (14). The mounting hole (15) can engage with the flange (49) on the driver gear (2) and rotates it in the same direction as the direction of rotation of the hinged cover (3). The boss (24) can protect and cover the driver gear (2). The edge (30) on the hinged cover (3) can act as a stopper to prevent further rotation of the hinged cover (3) once it is completely operated (actuated). The hinged cover (3) can also include the compartment (80) (See FIG. 38) to accommodate a power source and/or electronic instruments.

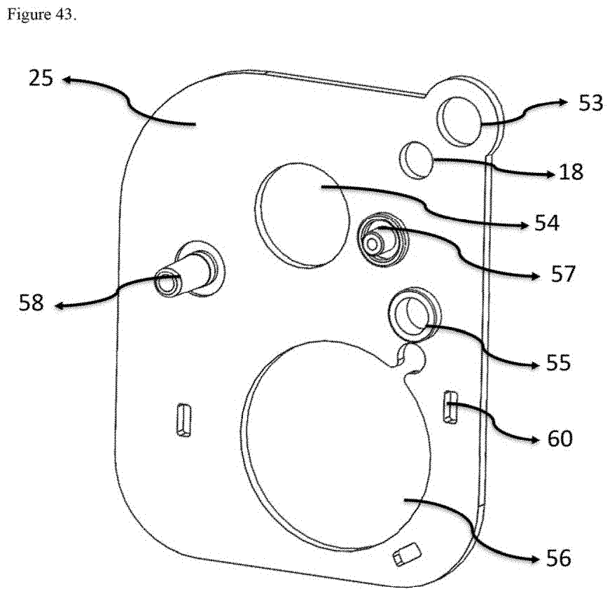

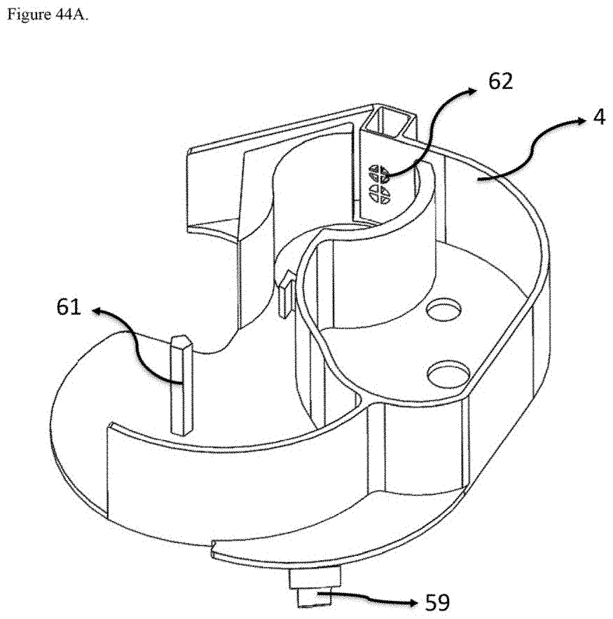

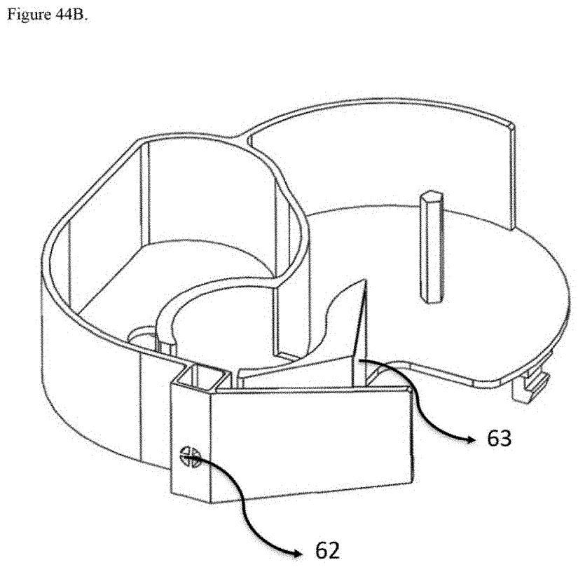

The inner housing support (25) (FIG. 43) can be mounted and permanently affixed to the base cover (14). The inner housing support (25) has grooves (60) that locate and fix the inner housing assembly (4).

The inner housing assembly (4) (FIGS. 44A-44B) can be locked and placed on the inner housing support (25). The locking features are the snapping hooks (59) that lock in the groove (60) of the inner housing support. Openings (53), (54), (55) and (56) allow and provide bearing to the driver gear (2), blister cuvette (8), wrapping wheel (11) and the dose indicator wheel (19) to pass through respectively. Stud (57) can locate the interim gear (10), and stud (58) can locate the winder assembly. Opening (18) can allow passage for the stud (45). The inner housing assembly (4) can hold the reel of blister (5) around the post (61). The inner housing assembly (4) provides the channels to guide the blister over the blister cuvette (8) as the reel of blister moves and peels during the process of operation/actuation of the medicament dispenser. The inner housing assembly (4) guides the two halves of the blister foil to wrap around the winder assembly (7) and the wrapping wheel (11). The inner housing assembly (4) can separate the rolled reel of blister (5) from the moving parts of the actuation mechanism. The inner housing assembly (4) also contains the mesh (62) to de-agglomerate the medicament when it leaves the blister during operation of the medicament dispenser. Guiding surface (63) can provide a frictionless surface for the reel of blister as it rolls on the winder assembly (7) during use.

The blister cuvette (8) (FIGS. 9A-9B) can locate each blister cavity (41) during the operation of the medicament dispenser. The blister cuvette (8) helps to locate and align the blister cavity along the flow channel/opening (12) when the mechanism is operated (actuated). The blister cuvette (8) can include eight equally spaced individual cuvettes (31) around the circumference. The cuvette ratchet (9) is located within the blister cuvette (8) and is responsible for discrete angular movement of the blister cuvette (8). The blister cuvette (8) rotates about the central boss (32) of the cuvette ratchet (9). The central boss (32) passes through and locates the central hole (33) on the blister cuvette (8). The spur gears (34) on the blister cuvette (8) can drive the winder assembly (7) and the wrapping wheel (11). The ratchets (35) can rest on the contacting surface (36) of the blister cuvette (8). The ratchets (35) can drive the blister cuvette (8) in one direction only through the contacting surface (36). When the direction of rotation of the cuvette ratchet (9) reverses while closing the medicament dispenser, the ratchets (35) ride on the non-contacting surfaces (37). The spur gears (39) on the cuvette ratchet (9) engage with the driver gear teeth (21).

The driver gear (2) (See FIG. 15) can be driven by the hinged cover (3) via the flange (49). The shaft (50) passes through and is guided by end boss (40) on the base cover (14) that passes through bearing (20) on the driver gear (2).

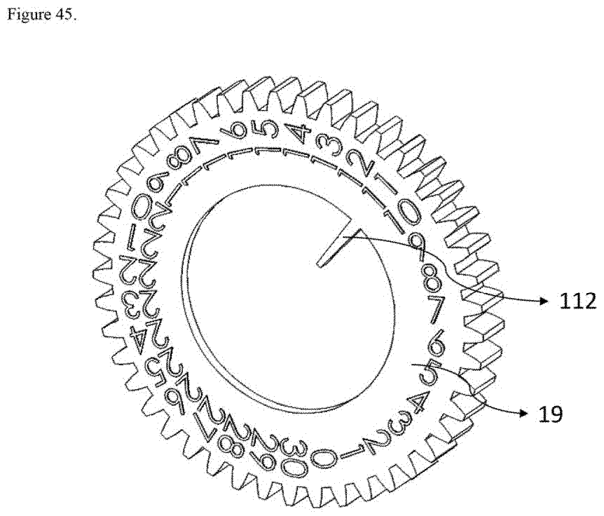

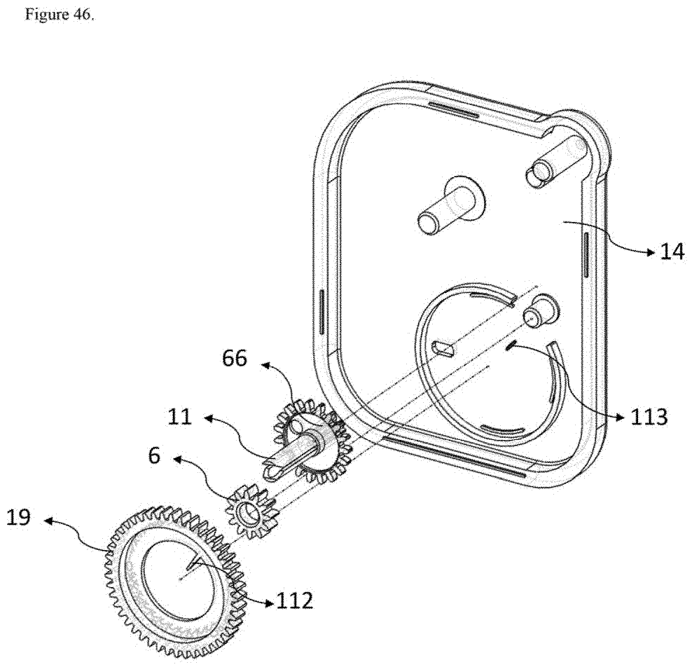

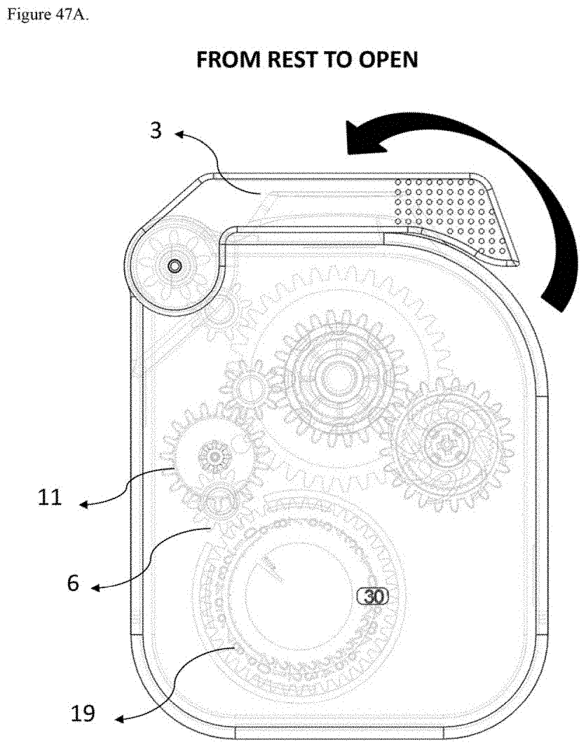

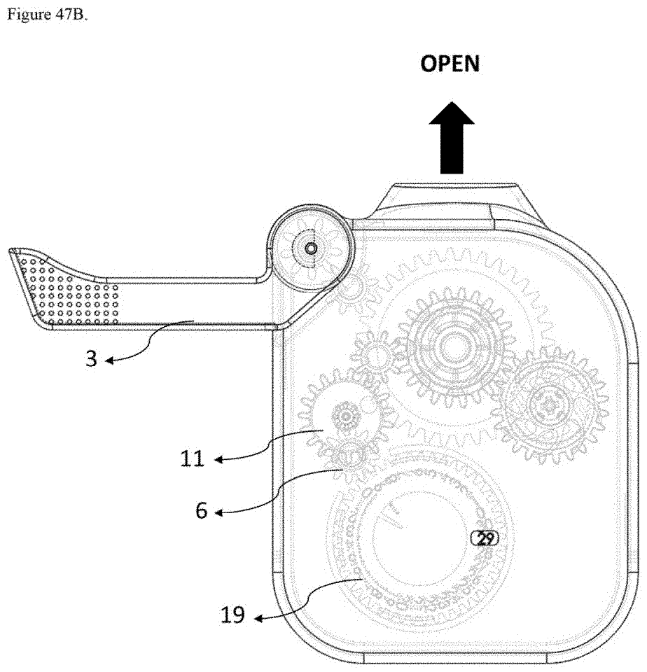

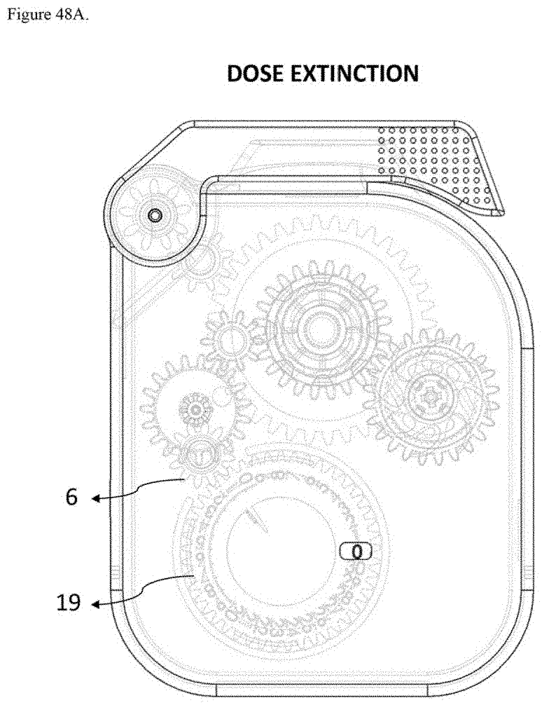

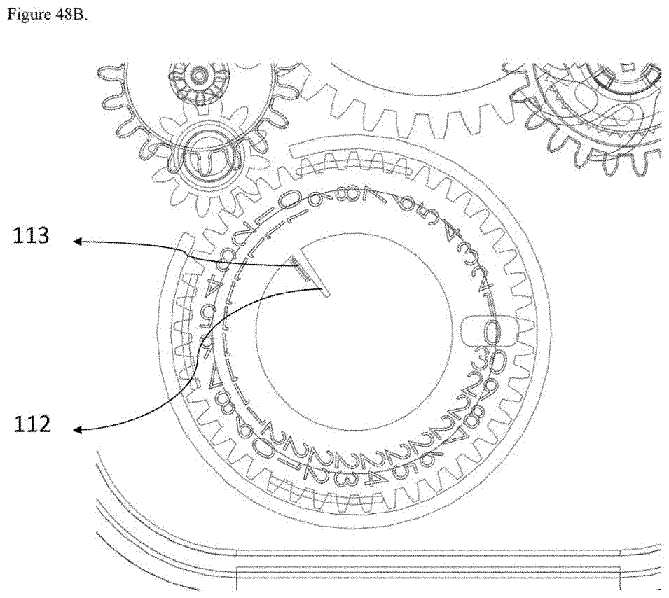

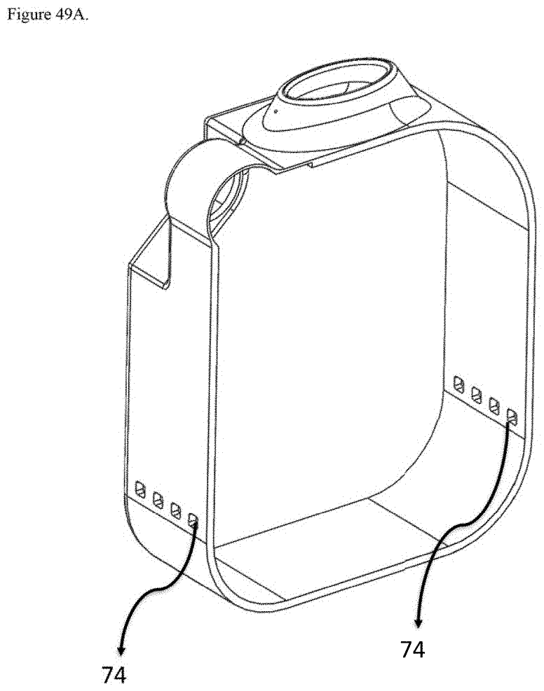

The dose indicator wheel (19) (FIGS. 32, 34B and 45) can be driven by the dose indicator driving gear (6). The dose indicator driving gear (6) in turn can be rotated by the gear (66) of the wrapping wheel (11). The dose indicator wheel moves incrementally displaying the number of doses remaining within the inhaler assembly. The dose numbers can be printed on surface. The dose indicator stopper (112) comes in contact with the dose indicator stopper stud (113) on the base cover (14) when the visible count reads ‘0’ and when all dose from the medicament dispenser are exhausted. At this stage the dose indicator mechanism and the blister advancing mechanism are locked.

The winder assembly (7) (FIGS. 16A-16B) can include the winder wheel (28), the winder gear (27) and the ratchet (26). The winder assembly (7) can be driven by the blister cuvette (8). Locking pins (51) can lock with the winder wheel (28). Sealed loop (70) of the reel of blister (5) can be located in blister post (38) of the ratchet (26). The reel of blister (5) is rolled on the winder wheel (28) during assembly and a pre-tension is provided by the rotation of the winder wheel (28). The assembly of the winder wheel (28) and the ratchet (26) ratchet on the inner gear (52) of the winder gear (27). Once the winder assembly (7) is locked, the winder wheel (28), winder gear (27) and the ratchet (26) rotate together.

The wrapping wheel (11) (FIG. 22) can be driven by the blister cuvette (8) via the spur gears (68). The open end (71) can be located with the slit (69) and wraps around the wheel as it rotates during actuation. Gear (66) drives the dose indicator driving gear (6) during actuation.

The wrapping wheel (11) rotates in the same direction of the blister cuvette (8) via the interim gear (10).

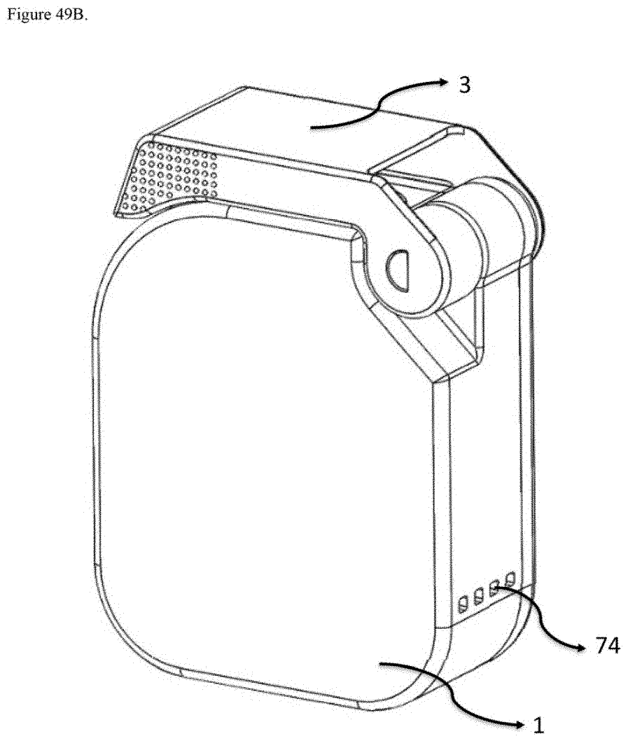

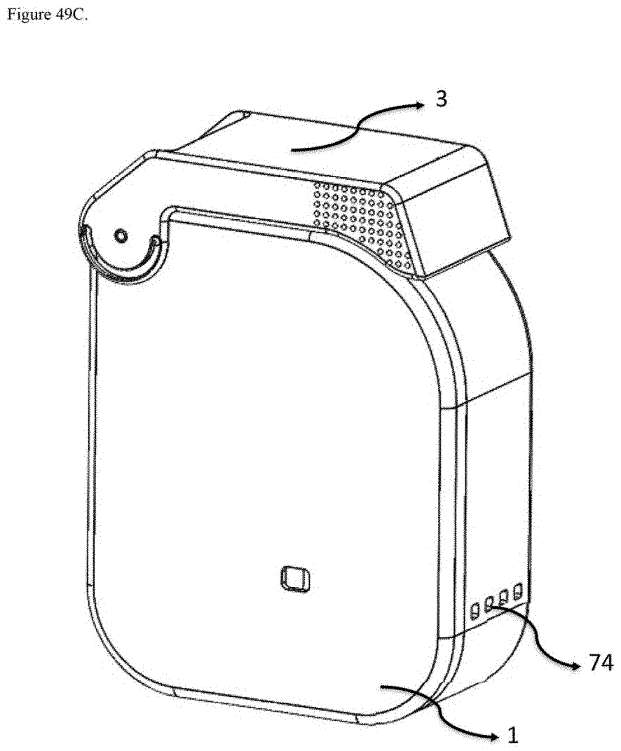

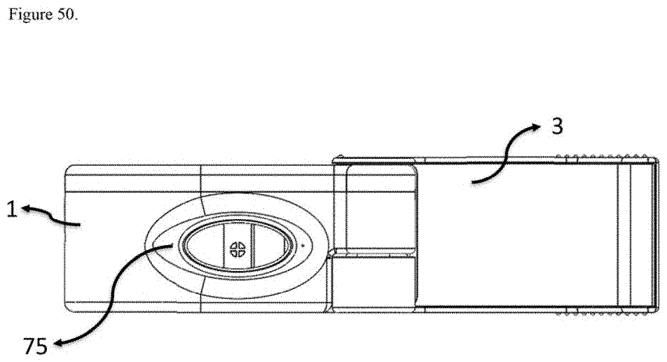

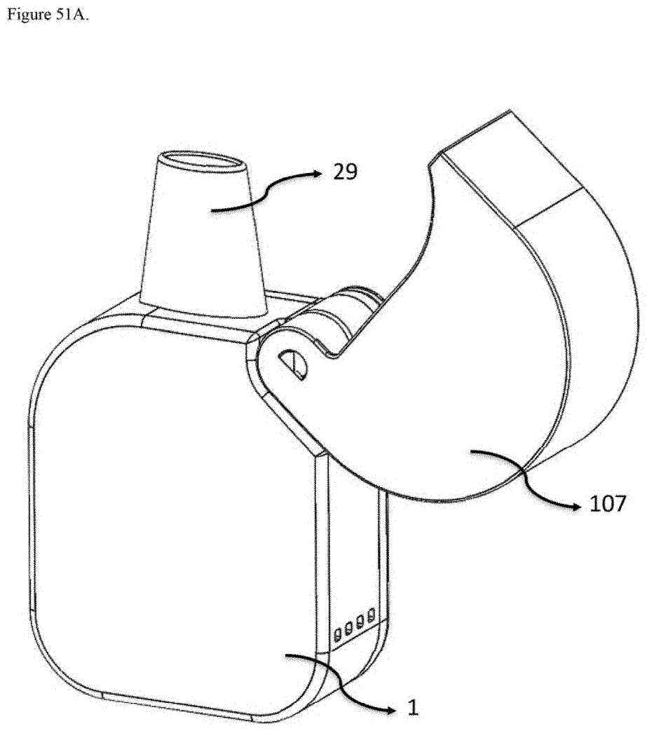

The air inlet vent holes (74) can be located discretely on the top cover to provide for an air inlet through the medicament dispenser. The mouthpiece vent holes (75) provide for by-pass air outlet (FIGS. 49A-49C and 50).

The reel of blisters (5) can incorporate a lid sheet (73), and a base sheet (72) which contains individual blister cavities (41) (FIGS. 26A-26B). The reel of blisters (5) can be wound around the post (61). One end of the reel of blister has a sealed loop (70) that can be placed in the blister post (38). The open end (71) of the reel of blister (5) can be placed in the slit (69) of the wrapping wheel (11).

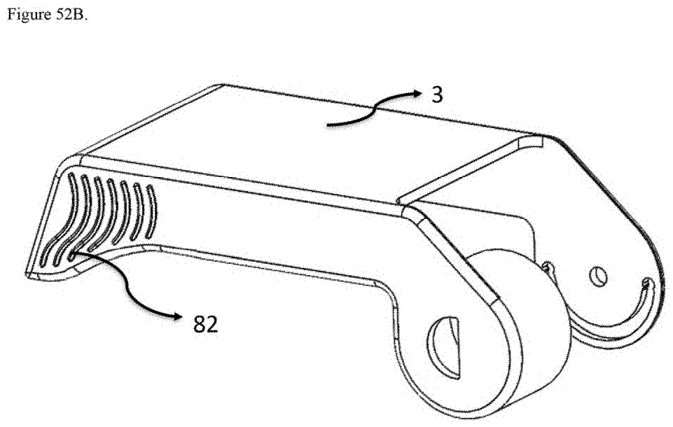

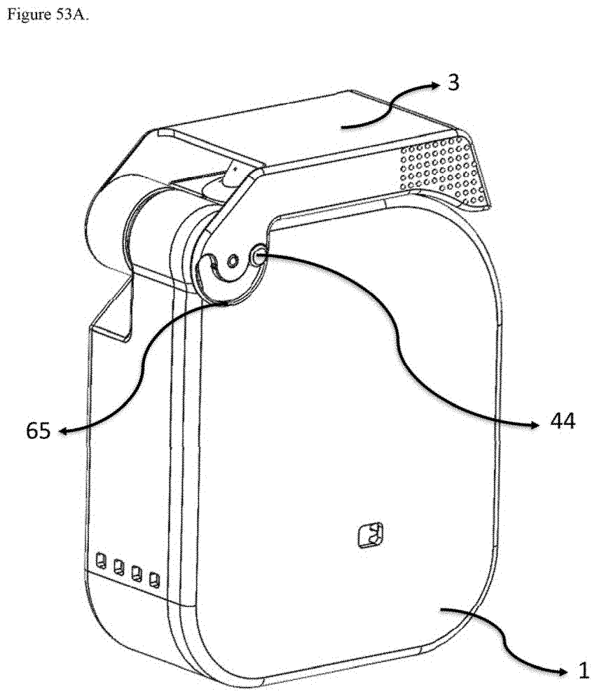

The dotted gripping feature (81) or the wave gripping feature (82) (FIGS. 52A-52B) can be provided for an efficient gripping mechanism to operate the hinged cover (3).

The window (48) provides a view of the dose indicator wheel (19) for user feedback on number of remaining doses.

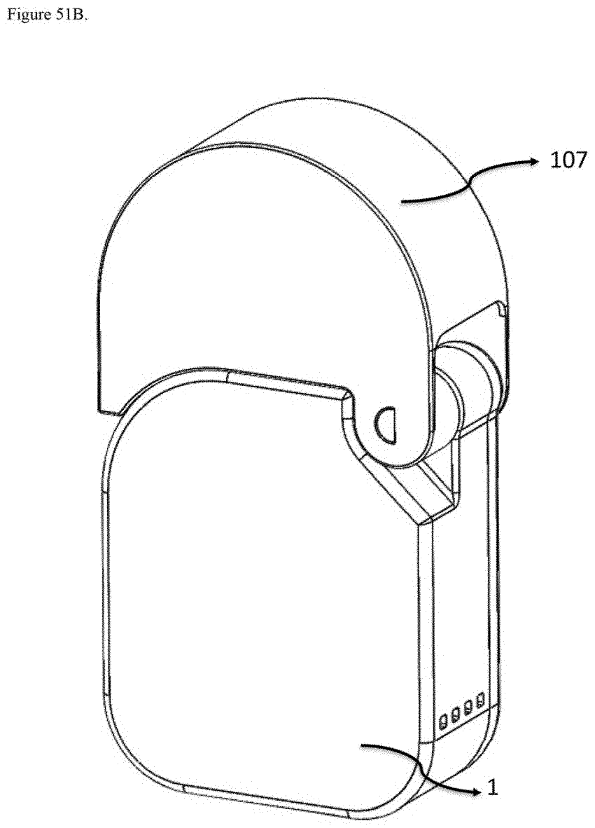

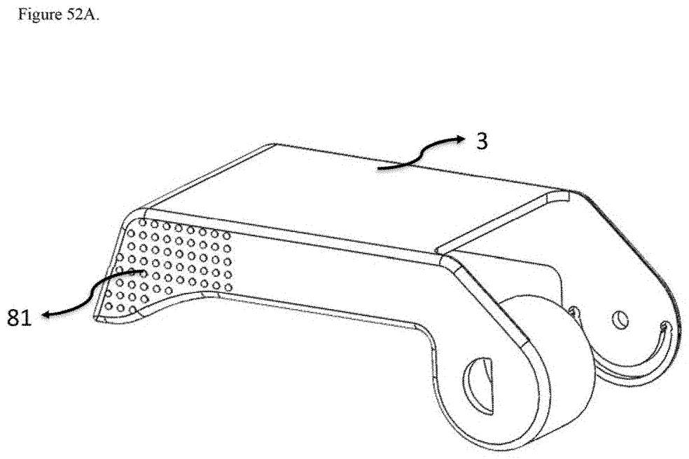

The mouthpiece (29) allows the user to receive the contents of the reel of blisters (5) by placing the mouthpiece (29) inside the oral cavity. The mouthpiece (29) allows the contents of the reel of blisters (5) to pass through the flow channel (12). The mouthpiece (29) can be covered by the hinged cover (3) when not in use. An extended mouthpiece (29) with corresponding hinged cover (107) can be provided to assist with de-agglomeration of the medicament and ease of delivery in the oral cavity (FIGS. 51A-51B).

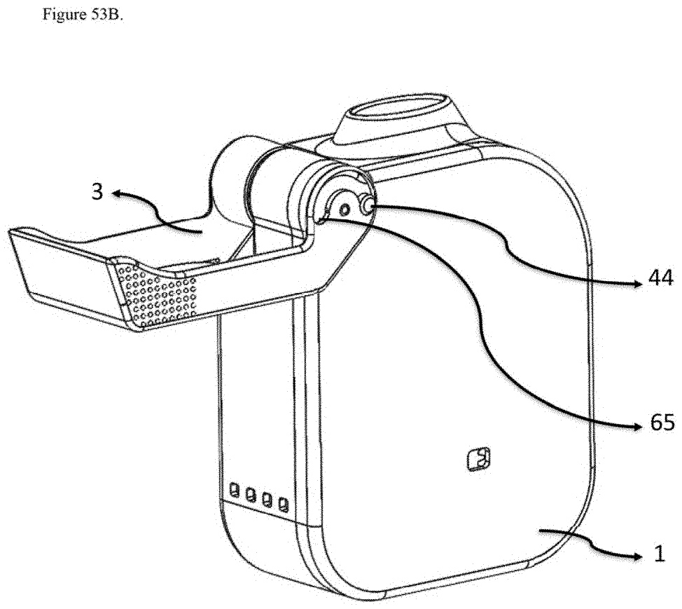

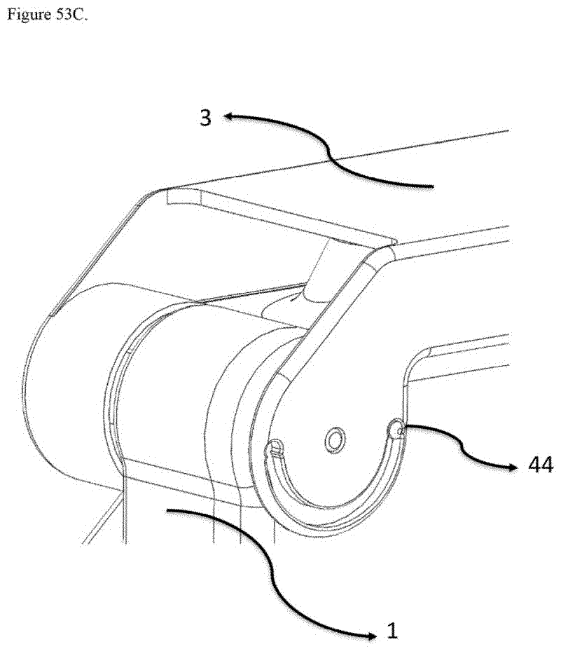

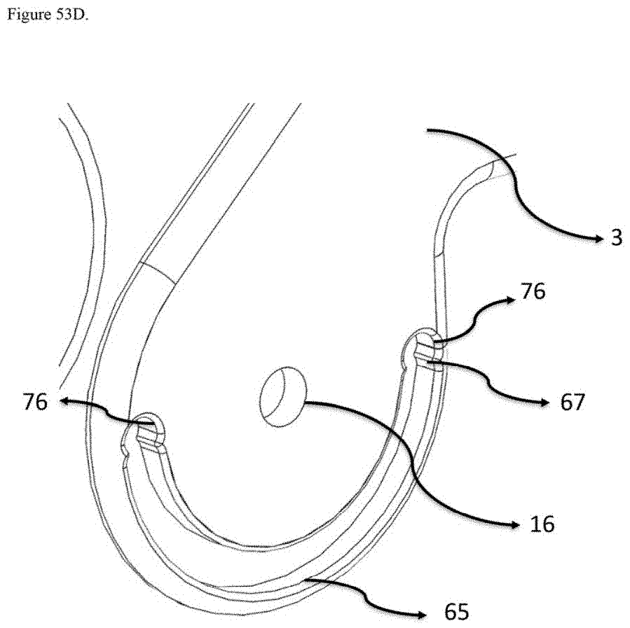

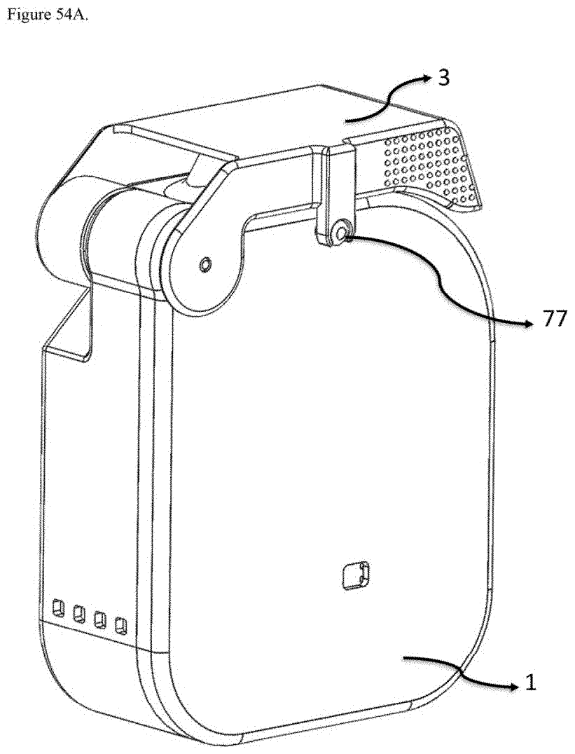

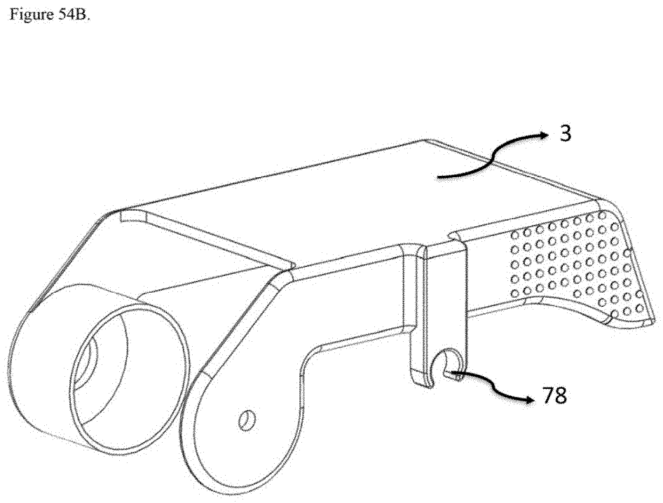

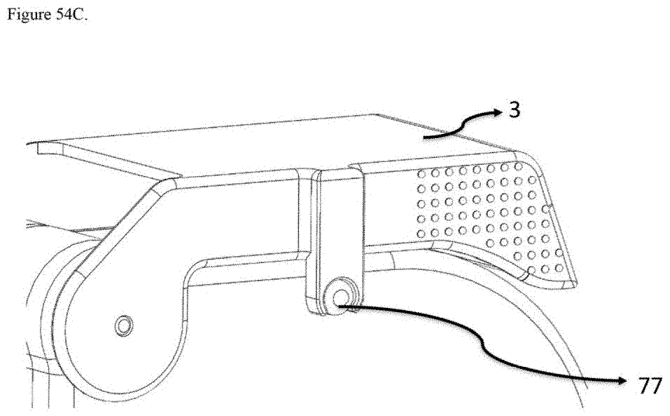

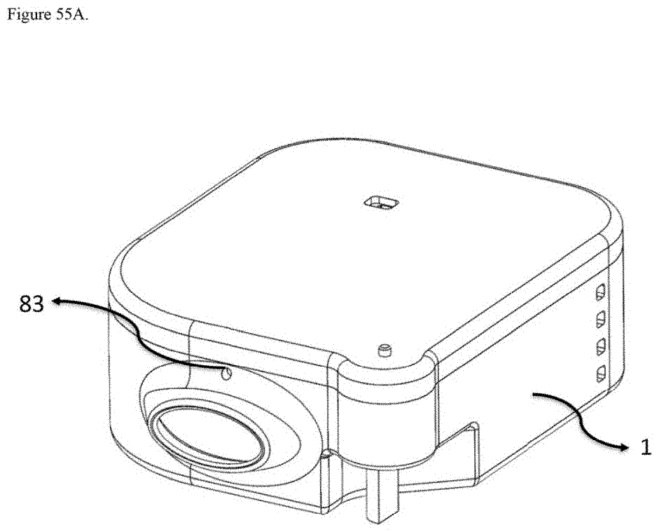

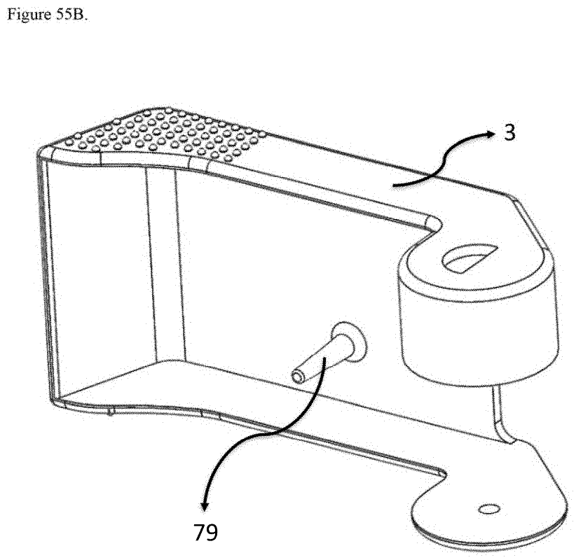

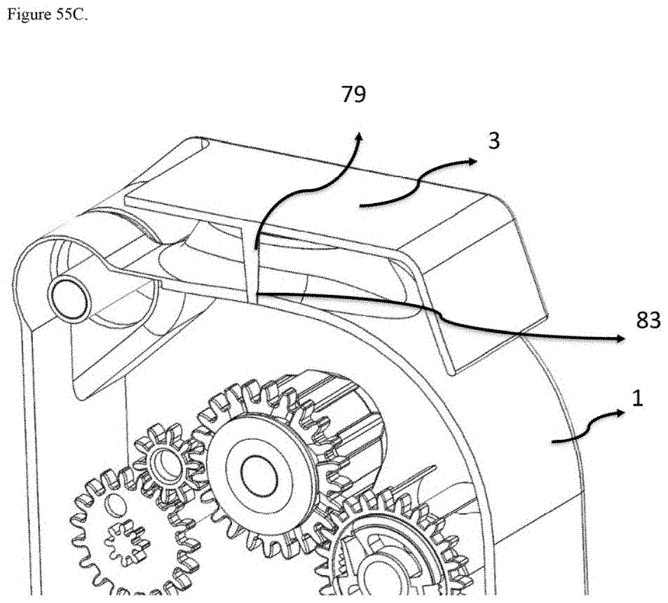

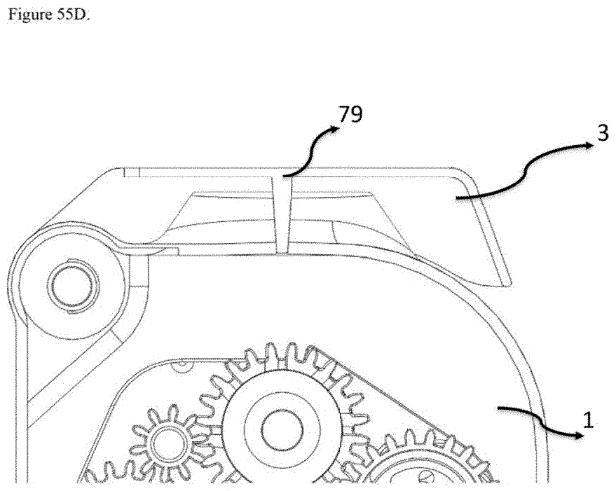

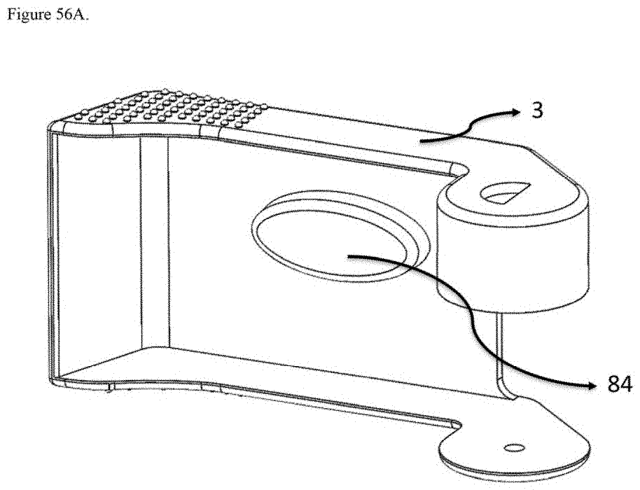

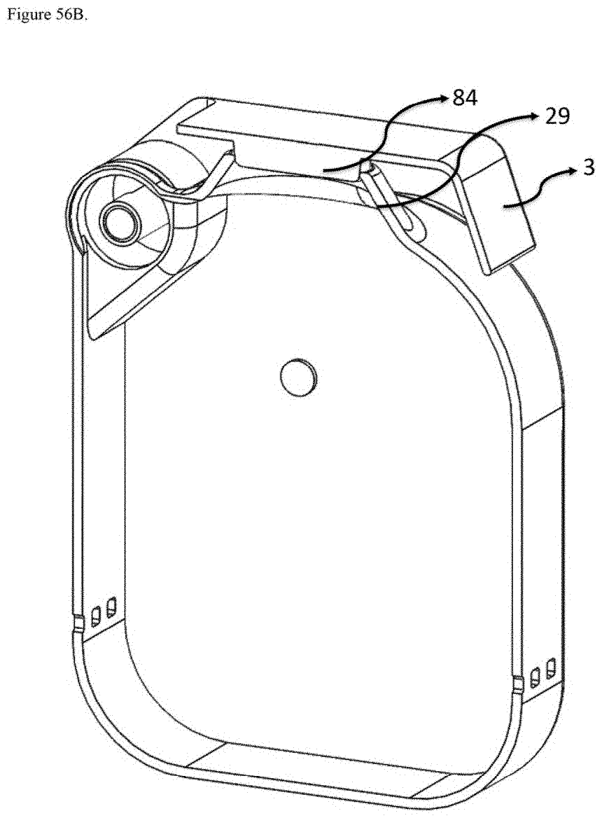

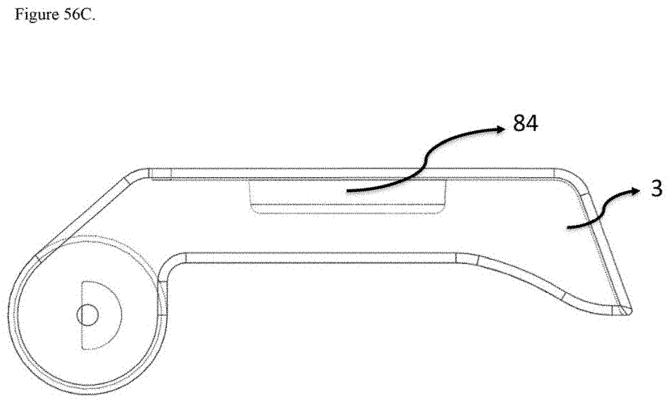

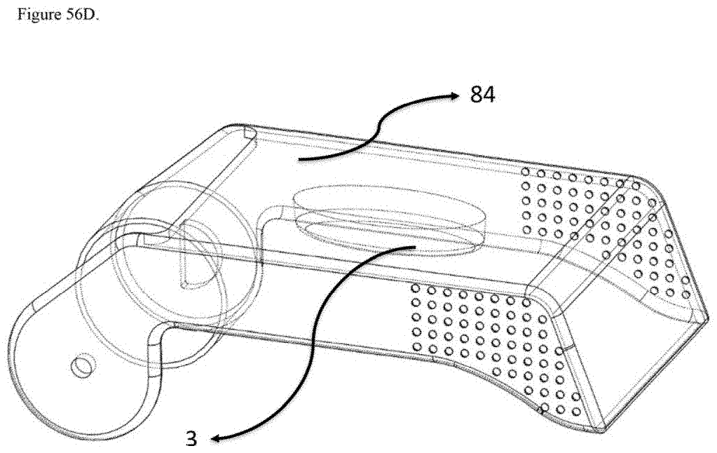

A locking/snapping mechanism for locking/snapping the hinged cover on the assembly can be provided. (FIGS. 53A-53D, 54A-54C, 55A, 55D, 56A-56D). The hinged cover (3) has a locking groove (65) that is guided by the locking stud (44) on the base cover (14). The locking notch (67) causes the locking stud (44) to snap within the locking space (76) both during opening and closing of the hinged cover (3). The dimensions of the locking groove (65), locking stud (44), locking notch (67) and locking space (76) can be modulated to provide a range of resistance values during the operation of the medicament dispenser. This mechanism prevents unintended and accidental operation/opening of the hinged cover. The snap provides an audible and haptic or kinesthetic feedback to the user indicating complete opening/actuation and complete closing of the medicament dispenser. The hinged cover (3) can have a locking groove (78) which can lock onto the locking stud (77) of the base cover. The snug fit between the locking groove (78) and the locking stud (77) provides resistance towards opening/actuation and closing of the hinged cover (3). The dimensions of the locking groove (78) and the locking stud (78) can be modulated to provide a range of resistance values during the operation of the medicament dispenser. This mechanism prevents unintended and accidental operation/opening of the hinged cover and provides haptic or kinesthetic feedback to the user indicating opening and closing of the medicament dispenser. The hinged cover (3) can have a locking pin (79) located on the inner surface that mates and locks with the locking vent (83) on the mouthpiece (29). The snug fit between the locking vent (83) and the locking pin (79) provides resistance towards opening/actuation and closing of the hinged cover (3). The locking vent (83) can also serve as the mouthpiece vent hole for air outlet. The dimensions of the locking pin (79) and locking vent (83) can be modulated to provide a range of medicament dispenser airflow resistance values and a range of resistance values during the operation of the medicament dispenser. This mechanism prevents unintended and accidental operation/opening of the hinged cover. The hinged cover (3) can have a projection (84) that mates with the opening of the mouthpiece (29). The snug fit between the locking projection (84) and the opening of the mouthpiece (29) provides resistance towards opening/actuation and closing of the hinged cover (3). This mechanism prevents unintended and accidental operation/opening of the hinged cover. This mechanism can also provide haptic or kinesthetic feedback to the user indicating opening and closing of the medicament dispenser. This mechanism can also provide for gentle tap on the mouthpiece of the medicament dispenser to dislodge any residual formulation on the mouthpiece and body of the medicament dispenser.

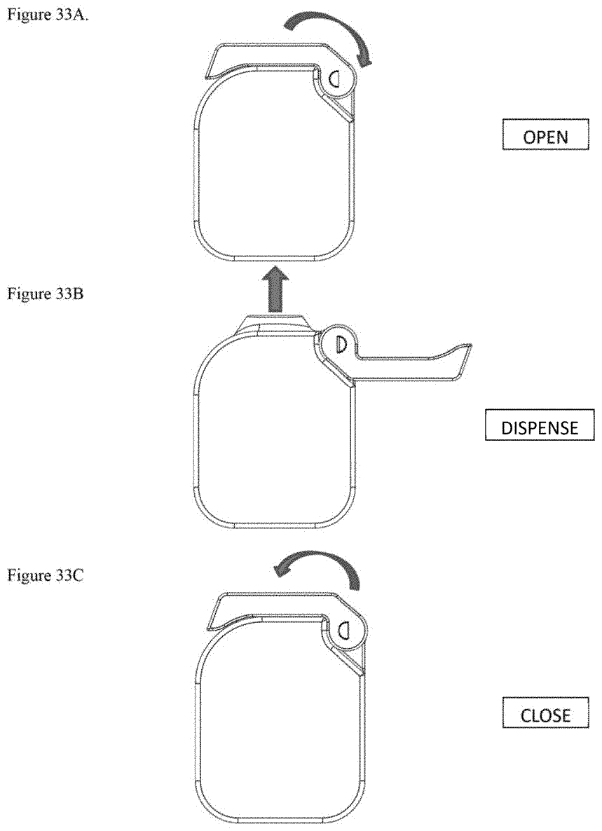

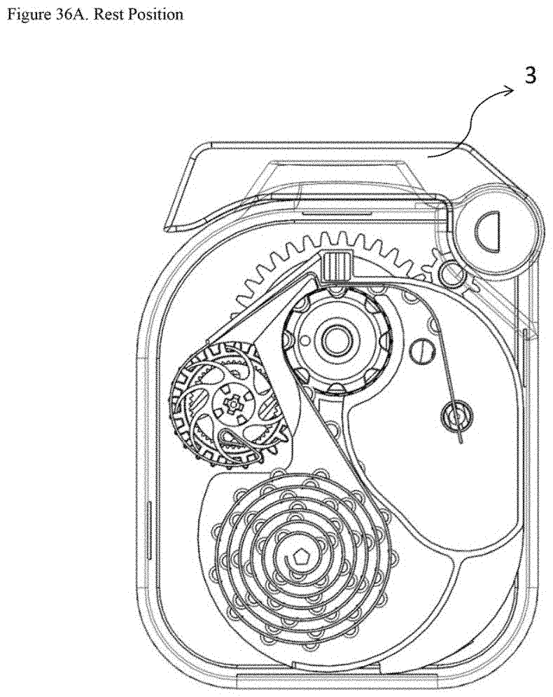

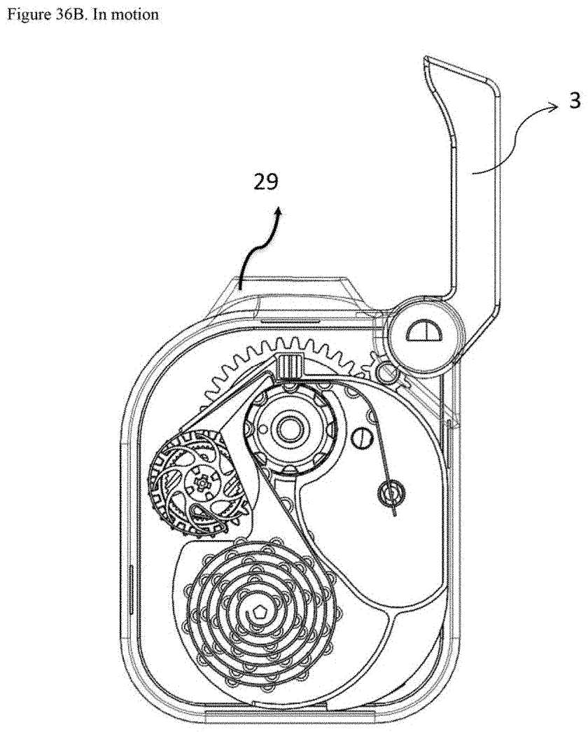

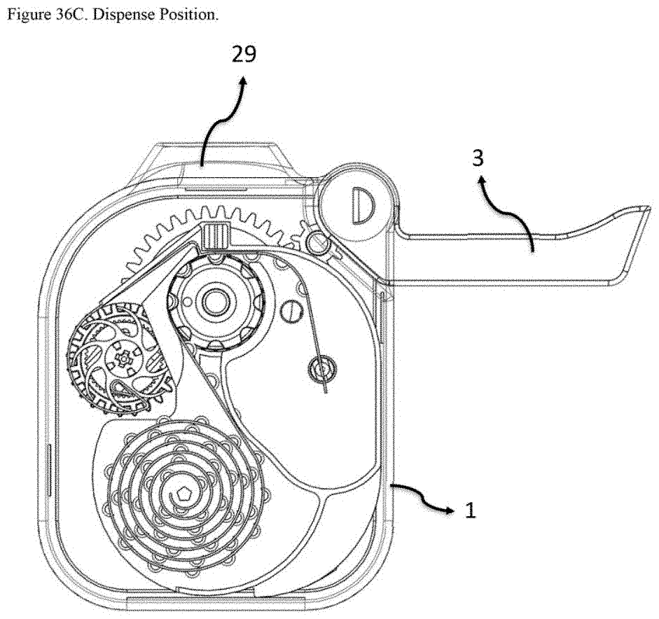

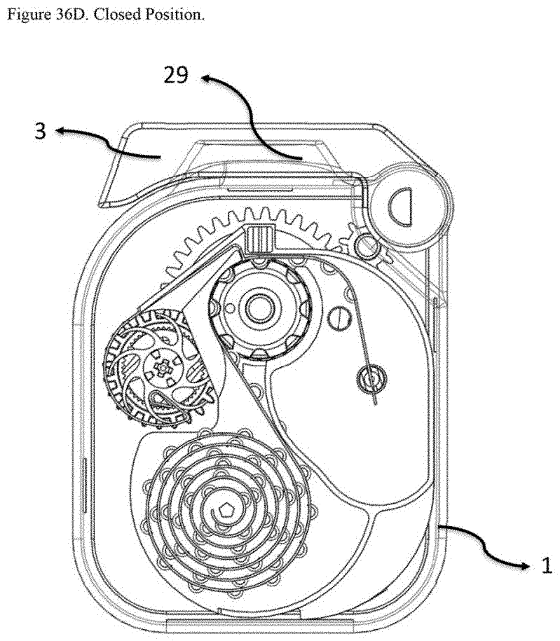

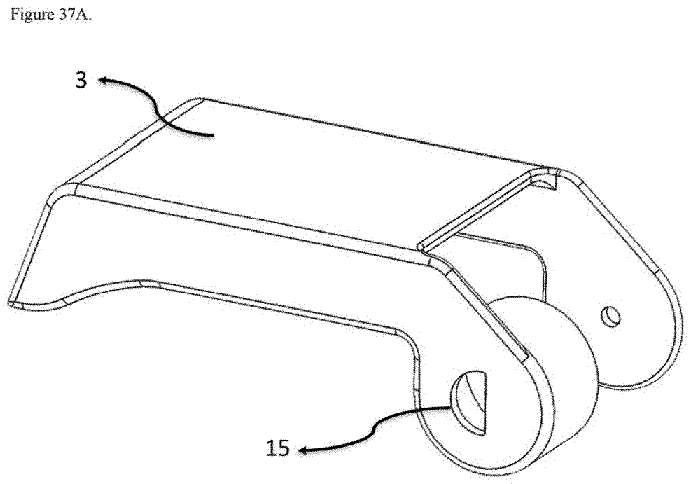

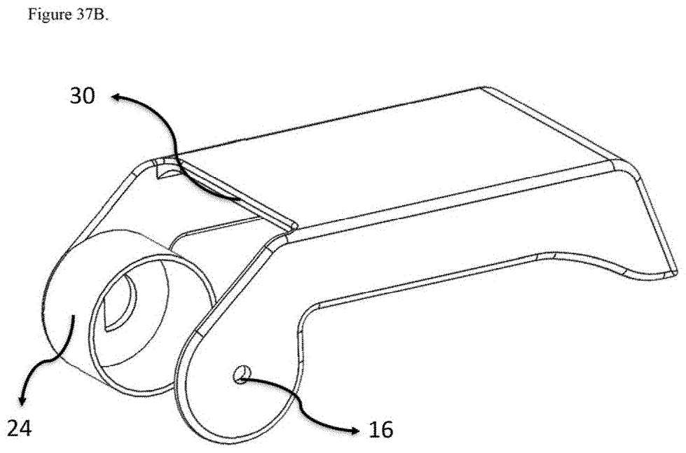

The mechanism of operation of the medicament dispenser is presented in FIGS. 33A-33C, 36A-36D. In the initial (rest) state the hinged cover (3) covers the flow channel (12) of the assembly (1). When the user opens (rotates) the hinged cover (3) by about 180°, the hinged cover (3) rotates the cuvette ratchet (9) via a driver gear (2) and an interim driver gear (17). The blister cuvette (8) rotates synchronously with the cuvette ratchet (9) uni-directionally, thereby advancing the blister cavities (41) from the reel of blisters (5). Advancement of reel of blisters (5) causes the leading blister cavity to be peeled open and brought into communication with the flow channel (12), which itself communicates with mouthpiece (29), and the medicament of the blister cavity are available for dispensing. To access the medicament of the opened cavities of reel of blisters the user places the mouth on the mouthpiece (29) and orally inhales. This results in air enters the device through the air inlet vent holes (74) and exits the device through the mouthpiece and through the mouthpiece vent holes (75). In turn, this results in the medicament contained within the opened cavities being aerosolized and guided through the flow channel and hence to the user as an inhaled medicament dose. When the user closes (rotates) the hinged cover back to initial (rest) state in the opposite direction, the motion of the hinged cover (3) rotates the cuvette ratchet (9) without advancing the reel of blisters. The ratcheting mechanism prevents the rotation of the blister cuvette (8) in the opposite direction.

The wrapping wheel (11) can rotate the dose indicator driving gear (6). During each actuation, the dose indicator driving gear (6) increments a single dose indicator wheel (19) in a serial manner, and the count is decreased from ‘30’ to ‘29’ and so forth. The remaining number of doses in the medicament dispenser are displayed in the window (48).

It will be appreciated that the above usage of the counter has been described in terms of a dose indicator driving gear arranged to count downwards, but that the dose indicator driving gear may be straightforwardly modified to count upwards.

The dose indicator stopper (112) comes in contact with the dose indicator stopper stud (113) of the base cover (14) when dose indicator wheel (19) reads the count ‘0’, and when all dose from the medicament dispenser are exhausted. At this stage the dose indicator mechanism and the blister advancing mechanism can be locked.

Conditional language used herein, such as, among others, “can,” “might,” “may,” “e.g.,” and the like, unless specifically stated otherwise, or otherwise understood within the context as used, is generally intended to convey that some embodiments include, while other embodiments do not include, certain features, elements, and/or states. Thus, such conditional language is not generally intended to imply that features, elements, blocks, and/or states are in any way required for one or more embodiments or that one or more embodiments necessarily include logic for deciding, with or without author input or prompting, whether these features, elements and/or states are included or are to be performed in any particular embodiment.

The ranges disclosed herein also encompass any and all overlap, sub-ranges, and combinations thereof. Numbers preceded by a term such as “about” or “approximately” include the recited numbers and values that are ±10%.

Although certain embodiments and examples have been described herein, it will be understood by those skilled in the art that many aspects of the medicament dispensers shown and described in the present disclosure may be differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

1. A medicament dispenser for dispensing medicament comprising:
a) an assembly comprising:
i) at least one peelable medicament carrier carrying multiple distinct medicament portions;
ii) a medicament carrier advancing mechanism;
iii) a flow channel; and
b) a hinged cover for the flow channel.

2. The medicament dispenser according to claim 1, wherein the hinged cover is movable between an initial position in which the flow channel is covered to a dispense position in which the flow channel is uncovered.

3. The medicament dispenser according to claim 2, wherein the movement of the hinged cover from the initial position results in actuation of the medicament carrier advancing mechanism.

4. The medicament dispenser according to claim 3, wherein the movement of the hinged cover from the initial position rotates a driver gear to actuate the medicament carrier advancing mechanism.

5. The medicament dispenser according to claim 1, wherein the actuation of the medicament carrier advancing mechanism rotates a cuvette ratchet and a blister cuvette synchronously and thereby advances the medicament carrier to align with the flow channel for dispensing the medicament.

6. The medicament dispenser according to claim 1, wherein the medicament dispenser comprises a plurality of said medicament carriers each carrying multiple distinct medicament dose portions.

7. The medicament dispenser according to claim 1, wherein the medicament comprises one or more active pharmaceutical ingredients and optionally, one or more pharmaceutically acceptable carriers.

8. The medicament dispenser according to claim 7, wherein the active pharmaceutical ingredient is selected from group consisting of anticholinergics, anti-inflammatories, analgesics, anginal preparations, anti-allergic, antihistamines, antitussives, bronchodilators, anti-infectives, anti-fungals, leukotriene inhibitors, PDE IV inhibitors, diuretics, hormones, cromolyns, xanthines, therapeutic proteins and peptides, vaccines, diagnostics and gene therapies or combinations thereof.

9. The medicament dispenser according to claim 1, wherein the medicament is in the form selected from group consisting of powder, tablet, capsule, pellet or puck, or mixtures thereof.

10. The medicament dispenser according to claim 1, wherein the dispenser is used for dispensing medicament for the treatment of asthma, chronic obstructive pulmonary disease (COPD), bronchitis, lung infections, interstitial pulmonary fibrosis (IPF), pulmonary arterial hypertension (PAH), cystic fibrosis, Non-CF Bronchiectasis or lung transplant.

11. The medicament dispenser according to claim 1, wherein the dispenser further comprises a dose counting or an indicating mechanism for counting or indicating number of doses released from the dispenser.

12. The medicament dispenser according to claim 11, wherein the dose counter is in the form of mechanical or non-mechanical.

13. The medicament dispenser according to claim 1, wherein the dispenser further comprises a resting surface.

14. The medicament dispenser according to claim 1, wherein the dispenser further comprises a power source, an electronic circuit and an electronic measurement instrument.

15. The medicament dispenser according to claim 1, wherein the dispenser is an inhalation device.

16. A medicament dispenser for dispensing medicament comprising:
a) an assembly comprising:
i) at least one peelable medicament carrier carrying multiple distinct medicament portions;
ii) a medicament carrier advancing mechanism;
iii) a flow channel; and
b) a hinged cover for the flow channel, wherein the flow channel is covered by the hinged cover in an initial position, and fully revealed when the hinged cover is in a dispense position.

17. The medicament dispenser according to claim 16, wherein the dispensing of the medicament provides continuous movement of the hinged cover from the initial position to the dispense position of the hinged cover without any intermediate position.

18. The medicament dispenser according to claim 16, wherein the dispensing of the medicament provides continuous movement of the hinged cover from the initial position to rotate a driver gear and actuate the medicament carrier advancing mechanism.

19. The medicament dispenser according to claim 16, wherein the dispensing of the medicament provides continuous movement of the hinged cover from the initial position results in actuation of the medicament carrier advancing mechanism.

20. The medicament dispenser according to claim 16, wherein the dispensing of the medicament does not provide any idle movement of the hinged cover.

21. A medicament dispenser for dispensing medicament comprising:
a) an assembly comprising:
i) at least one peelable medicament carrier carrying multiple distinct medicament portions;
ii) a medicament carrier advancing mechanism;
iii) a flow channel;
iv) a driver gear; and
b) a hinged cover for the flow channel.

22. A medicament dispenser for dispensing medicament comprising:
a) an assembly comprising:
i) at least one peelable medicament carrier carrying multiple distinct medicament portions;
ii) a medicament carrier advancing mechanism;
iii) a flow channel;
iv) a driver gear;
v) an interim driver gear; and
b) a hinged cover for the flow channel.
